(19) United States
(12) Patent Application Publication
(10) Pub. No.: US 2007/0218480 A1
(43) Pub. Date: Sep. 20, 2007

(54) DETECTION AND DIAGNOSIS OF SMOKING RELATED CANCERS

(75) Inventors: Ruth L. Katz, Houston, TX (US); Feng Jiang, Ellicott City, MD (US)

Correspondence Address: FULBRIGHT & JAWORSKI L.L.P., 600 CONGRESS AVE., SUITE 2400, AUSTIN, TX, 78701, US

(73) Assignee: THE BOARD OF REGENTS OF THE UNIVERSITY OF TEXAS SYSTEM

(21) Appl. No.: 11/627,253

(22) Filed: Jan. 25, 2007

Related U.S. Application Data
(60) Provisional application No. 60/761,806, filed on Jan. 25, 2006.

(51) Int. Cl. C12Q 1/68

(52) U.S. Cl. 435/6

(57) ABSTRACT

Gene probes for specific regions of chromosomes 1, 3, 9, 10 and 17 are now shown to be useful in the diagnosis and prognosis of smoking related cancers such as non-small cell lung cancer (NSCLC). For example, these probes can be used with fluorescence in situ hybridization (FISH), and used to stratify smokers into high and low risk groups, to determine susceptibility to the development of smoking related cancers, to predict cancer progression and treatment efficacy, and to rule out other diseases.

The present application claims benefit of priority to U.S. Provisional Application Ser. No. 60/761,806, filed Jan. 25, 2006, the entire contents of which are hereby incorporated by reference.

The government may own rights to this invention pursuant grant no. RFP N01 CN 85083 57 from the National Institutes of Health.

### BACKGROUND OF THE INVENTION

I. Field of the Invention

The present invention relates to the fields of oncology, genetics and molecular biology. More particular the invention relates to the use of multiple probes for regions of genetic instability that are highly predictive of the development of neoplasia and progression of neoplastic events.

II. Related Art

Lung cancer is one of the leading causes of cancer death in the world. The high mortality rate for lung cancer probably results, at least in part, from the lack of standard clinical procedures for the diagnosis of the disease at early and more treatable stages compared to breast, prostate, and colon cancers. There is also extremely poor prognosis associated with diagnosis of the disease, especially in advanced disease. It is important that strategies to detect early stage lung carcinoma or its precursors, such as atypical squamous metaplasia, dysphasia and carcinoma-in-situ in subjects at high risk be devised.

Cigarette smoking over a prolonged period of time is the most important risk factor in the development of lung and other smoking related cancers, with other risk factors including exposure to passive smoking, certain industrial substances such as arsenic, some organic chemicals, radon and asbestosis, ingestion of alcohol, radiation exposure from occupational, medical and environmental sources, air pollution and tuberculosis. Many of these factors greatly increase the risk of development of lung and other smoking related cancers if they occur in a person who is concurrently a smoker.

Genetic detection of human disease states is a rapidly developing field (Taparowsky et al., 1982; Slamon et al., 1989; Sidransky et al., 1992; Miki et al., 1994; Dong et al., 1995; Morahan et al., 1996; Lifton, 1996; Barinaga, 1996). However, some problems exist with this approach. A number of known genetic lesions merely predispose to development of specific disease states. Individuals carrying the genetic lesion may not develop the disease state, while other individuals may develop the disease state without possessing a particular genetic lesion. In human cancers, genetic defects may potentially occur in a large number of known tumor suppresser genes and proto-oncogenes.

The genetic detection of cancer has a long history. One of the earliest genetic lesions shown to predispose to cancer was transforming point mutations in the ras oncogenes (Taparowsky et al., 1982). Deletion and mutation of p53 has been observed in bladder cancer (Sidransky et al., 1991). Numerous studies have shown deletions in the 3p region are related to lung and other smoking related cancers (Mitsudomi et al., 1996, Shiseki et al., 1996, Wistuba et al., 2000, Wu et al., 1998, and Shriver et al., 1998).

Molecular studies (fluorescence in situ hybridization (FISH) for polysomies, PCR™ for hypervariable markers (MI) and LOH, or specific mutations) have demonstrated that morphologically normal areas of bronchial epithelium closest to the carcinomas frequently show the most molecular abnormalities (3p, 17p, 9p, 5q). In particular, the short arm of chromosome 3 has been shown to frequently harbor deletions of alleles in several regions including 3p25-26, 3p21.3-22, 3p14 and 3p12. These regions are presumed to be the site of tumor suppressor genes, and loss of chromosome 3p allelles have shown to be an early event in lung tumorigenesis.

Chromosomal alterations in several cancers have been investigated, and frequent LOH at chromosome 10 has been reported in a variety of cancers, including glioma, glioblastoma multiforme, prostate cancer, endometrial cancer, chondrosarcome, bladder cancer, malignant melanoma, and follicular thyroid tumors ((Licciardello et al., 1989; Auerbach et al., 1961; Voravud, et al., 1993; Feder et al., 1998; Yanagisawa et al., 1996; Thiberville et al., 1995; Papadimitrakopoulou et al., 1996; Zou et al., 1998; Brugal et al., 1984; Dalquen et al., 1997; Muguerza et al., 1997).

Deletion rates of chromosome 3p are known to correlate with lung cancer. However, there is no current clinical method for the identifying a population of individuals who are at a high risk to develop lung cancers or upper airway primary or secondary cancers. A technique for determining the risks of developing these cancers would be of great value for the ability to limit exposure to additional environmental risk factors and to know when additional tests, supplements, or treatments are appropriate.

In various studies, chromosome deletions have been studied as identifiers for lung cancers. For example, Shiseki et al. (1996) analyzed 85 loci on all 22 autosomal chromosomes to determine that the incidence of LOH on chromosome arms 2q, 9p, 18q, and 22q in brain metastases were significantly higher than that in stages I primary lung tumors. Mitsudomi et al. (1996) used PCR-based analysis for the detection of LOH in non-small cell lung cancer. Multiple regions on chromosome 3p were observed to show that deletions of the 3p chromosome may help to identify non-small cell lung cancer patients with a poor prognosis. Wistuba et al. (2000) used fifty-four polymorphic markers used to study the entire chromosome arm 3p and concluded that 3p allele loss is nearly universal in lung cancer pathogenesis. Wu et al. (1998) studied 3p21.3 deletion using the probe, D3S4604/luca. Peripheral blood lymphocytes of 40 lung cancer patients were observed to give the conclusion that lung cancer patients exposed to benzo[α]pyrene, a common byproduct of tobacco smoke, have frequent deletions in peripheral blood lymphocytes. Shriver et al. (1998) studied lung cancer cell lines and identified the human homolog of the L14 ribosomal protein gene, RPL14; deletion of RPL14 was shown to be related to the development of lung cancer. None of theses studies, however, are able to predict the susceptibility of a patient to the development of lung cancer or to predict whether smokers and non smokers are at a high risk of developing lung or other smoking related cancers.

Previously, the inventors have reported on the use of gene probes for specific regions of chromosome 3 (3p21.3) and chromosome 10 (10q22) in the diagnosis and prognosis of smoking related cancers such as non-small cell lung cancer (NSCLC). U.S. Pat. No. 6,797,471. These probes were used with fluorescence in situ hybridization (FISH) to stratify smokers into high and low risk groups, as well as determine a patient's susceptibility to the development of smoking related cancers.

Because of the grim prognosis of lung cancer with a ten year survival rate of <5% the only curable cancers are those diagnosed in the early stages and treated surgically. There is a shift of interest towards diagnosis and study of early and preneoplastic states. Because early detection and effective chemoprevention therapy have potential to be curative, it is imperative to stratify the patients in clinical trials. These patients need to be monitored fore results of chemoprevention therapy and also for predictions whether a particular preneoplastic lesion may progress.

### SUMMARY OF THE INVENTION

Thus, in accordance with the present invention, there is provided a method for identifying a subject at risk for the development of cancer comprising (a) providing probes for cen3 and cen17; (b) contacting said probes with a nucleic acid test sample from said subject; and (c) analyzing the hybridization pattern of said probes to said nucleic acid test sample, whereby aberrations in the hybridization of said probes to said nucleic acid test sample, as compared to a normal nucleic acid sample, indicates that said subject is at risk for the development of cancer. The method may further comprise (i) providing a probe for cen 1; (ii) contacting said cen 1 probe with a nucleic acid test sample from said subject; and (iii) analyzing the hybridization pattern of said cen 1 probe to said nucleic acid test sample. The method may also further comprise analyzing the hybridization pattern of one or more probes for 3p22.1, 1 q21, 9p21.3, 10 q22, cen7 or cen10.

The test sample may comprise a surgical or biopsy specimen, a paraffin embedded tissue, a frozen tissue imprint, sputum, a lavage, peripheral blood, a bladder washing or barbotage, renal pelvic brushes, conduit urine, voided urine, esophageal brush, a fine needle aspirate, a buccal smear, spinal fluid, or serous cavity effusions such as pleural fluid or ascites. The cancer may be lung cancer, or more specifically, non-small cell lung cancer or small cell lung cancer. The cancer may be an upper airway primary or secondary cancer. The cancer may also be a bladder cancer, a head and neck cancer, a urothelial cancer, a cancer of the kidneys, a cancer of the pancreas, or a cancer of the mouth, throat, pharynx, larynx, or esophagus. The subject may be a smoker, a former smoker, or a non-smoker. The subject may not previously have been diagnosed with cancer. The probe may be labeled with a fluorophore. The probe may be between 100,000 and 300,000 base pairs.

The method may further comprise a spiral CT-scan or an endoscopic evaluation of the bronchial tree of said subject. The method may also further comprise administering to said subject chemopreventive drugs, nutritional supplements, cytokine, radiation, chemotherapeutic drugs or biological modifying response drugs, gene therapy, siRNA therapy or stem cells. The method may also further comprise making a decision on whether said subject is in need of an intensive follow-up protocol. The method may also further comprise making a determination of whether said subject is responding to a therapy.

Analyzing may comprise FISH. The nucleic acid test sample may be subject to separation on the basis of cell type prior to step (b). The abberations in hybridization may be caused by deletions, amplifications or polysomies in regions corresponding to said probes. The cell type may comprise cancer cells, lymphocytes, monocytes, histiocytes, neutrophils (including granulocytes) and/or epithelial cells. The method may further comprise taking a patient history. The patient history may comprise smoking history, presence or absence of morphologic changes in sputum morphology (squamous metaplasia, dysplasia, etc.) and a genetic instability score.

In another embodiment, there is provided a method for identifying a subject at risk for the recurrence of cancer comprising (a) providing probes for 3p22.1, 1q21, 9p21.3, 10q22, cen17, and one or more of cen1, cen3, cen9, and cen10; (b) contacting said probes with a nucleic acid test sample from said subject; and (c) analyzing the hybridization pattern of said probes to said nucleic acid test sample, whereby aberrations in the hybridization of said probes to said nucleic acid test sample, as compared to a normal nucleic acid sample, indicates that said subject is at risk for the recurrence of cancer.

In yet another embodiment, there is provided a method for identifying a subject at risk for metastatic cancer comprising (a) providing probes for 1q21, 3p22.1, 9p21.3, 10q22, cen17, and one or more of cen1, cen3, cen9 and cen10; (b) contacting said probes with a nucleic acid test sample from said subject; and (c) analyzing the hybridization pattern of said probes to said nucleic acid test sample, whereby aberrations in the hybridization of said probes to said nucleic acid test sample, as compared to a normal nucleic acid sample, indicates that said subject is at risk for metastatic cancer.

In still yet another embodiment, there is provided a method for predicting cancer progression in a subject comprising (a) providing probes for 3p22.1, 1q21, 10q22, 9p21.3, cen17, and one or more of cen1, cen3, cen9, and cen10; (b) contacting said probes with a nucleic acid test sample from said subject; and (c) analyzing the hybridization pattern of said probes to said nucleic acid test sample, whereby aberrations in the hybridization of said probes to said nucleic acid test sample, as compared to a normal nucleic acid sample, indicates that said subject will suffer from progressive cancer. The patient having alterations in 10q22 may be treated with chemotherapy.

In a further embodiment, there is provided a method for distinguishing cancer from granulomatous disease in a subject comprising (a) providing probes for 3p22.1, 1q21, 9p21.3, 10q22, cen17, and one or more of cen1, cen3, cen9, and cen10; (b) contacting said probes with a nucleic acid test sample from said subject; (c) analyzing the hybridization pattern of said probes to said nucleic acid test sample; and (d) comparing the hybridization patterns in nucleic acids from two more more cell types represented in said nucleic acid test sample, said two or more cell types comprising neutrophils and epithelials cells, whereby aberrations in the hybridization of said probes to said neutrophil nucleic acids in said nucleic test sample indicates granulomatous disease, and aberrations in the hybridization of said probes to said epithelial cell nucleic acids in said nucleic test sample indicates cancer.

In still a further embodiment, there is provided a method of identifying a subject to be segregated from a high cancer risk environment comprising (a) providing probes for 3p22, 1q21, 9p21.3, 10q22, cen17, and either cen1, cen3 or cen9, cen10; (b) contacting said probes with a nucleic acid test sample from said subject; and (c) analyzing the hybridization pattern of said probes to said nucleic acid test sample, whereby aberrations in the hybridization of said probes to said nucleic acid test sample, as compared to a normal nucleic acid sample, indicates that said subject should be segregated from said high cancer risk environment.

In yet another embodiment, there is provided a method of identifying a subject who may be at lower risk to develop cancer of the aerodigestive tract and therefore may continue to use tobacco products comprising (a) providing probes for 3p22.1, 1q21, 9p21.3, 10q22, cen17, and one or more of cen1, cen3 cen9, and cen10; (b) contacting said probes with a nucleic acid test sample from said subject; and (c) analyzing the hybridization pattern of said probes to said nucleic acid test sample, whereby a lack of aberrations in the hybridization of said probes to said nucleic acid test sample, as compared to a low-risk nucleic acid sample, indicates that said subject may continue to use tobacco products.

In still a further embodiment, there is provided a method of assessing a cancer therapy or chemopreventative therapy in a subject comprising (a) providing probes for 3p, 10q and cen3; (b) contacting said probes with a nucleic acid test sample derived from epithelial cells and neutrophils from said subject; and (c) assessing the amount of aneusomies on chromosome 10, 3p deletions and aneusomies of centromeric 3; wherein a decrease in the amount of aneusomies on chromosome 10, 3p deletions and aneusomies of centromeric 3 indicates an effective treatment or prevention.

It is contemplated that any method or composition described herein can be implemented with respect to any other method or composition described herein.

The use of the word “a” or “an” when used in conjunction with the term “comprising” in the claims and/or the specification may mean “one,” but it is also consistent with the meaning of “one or more,” “at least one,” and “one or more than one.”

These, and other, embodiments of the invention will be better appreciated and understood when considered in conjunction with the following description and the accompanying drawings. It should be understood, however, that the following description, while indicating various embodiments of the invention and numerous specific details thereof, is given by way of illustration and not of limitation. Many substitutions, modifications, additions and/or rearrangements may be made within the scope of the invention without departing from the spirit thereof, and the invention includes all such substitutions, modifications, additions and/or rearrangements.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein:

FIGS. 1A-C. An SP-A specific probe. FIG. 1A. Schematic SP-A gene probe of approximately 110 kb was mapped to cover both SP-A1 and SP-A2. FIG. 1B. Dual-color FISH analysis of a normal metaphase spread with the SP-A gene specific probe (green) and chromosome 10 centromeric probe (CEP10) (red), which is used as control. The green signals indicate that the location of the probe is on chromosome 10q22-3. FIG. 1C. Dual-color FISH analysis of a normal lung tissue with the same pair of probes. Nuclei show two SP-A signals (green) and two (CEP10) probe signals (red).

FIGS. 2A-C. Micro-dissection guided FISH analysis of lung cancer specimens. FIG. 2A representative tumor and bronchioles were located microscopically and marked on hematoxylin-eosinstained sections. Corresponding areas were then identified on the paraffin blocks, and core tissue samples were taken from each block. FIG. 2B. Dual-color FISH analysis of isolated nuclei from adjacent bronchial tissue cells shows deletion of SP-A gene: few green signals (two green dots) than red signals (three red dots). FIG. 2C. The FISH analysis of isolated nuclei from tumor cells shows a high level of deletion of SP-A gene: two SP-A signals and four or more CEP10 signals.

FIG. 3. SP-A deletion status occurred in normal appearing adjacent bronchial cells to tumors in stage I non-small-cell lung cancer and probability of disease-specific survival time. The Kaplan-Meier method was used to determine the survival probability, and the log-rank test was used to compare the survival curves between patients with SP-A deletion and patients without SP-A deletion. A specimen with a SP-A deletion was defined when ≧4% of cells show deletion of SP-A.

FIGS. 4A-S. Blip Plot of Patients Characteristics grouped by Cancer Status. Patients with cancer are depicted by a top row of small circles, controls by bottom row. Variables depicted are as follows (from left to right and sequentially down the page): FIG. 4A—Age; FIG. 4B—Pack Year smoking; FIG. 4C—Number of cigarettes smoked per day; FIG. 4D—Number of years of smoking; FIG. 4E—Deletions of 3p22.1 compared to centromeric 3, in epithelial cells; FIG. 4F—Polysomies or additional chromosomes >2 for centromere of chromosome 3; FIG. 4G—Deletions of 10q22-23, the gene for surfactant protein A in epithelial cells; FIG. 4H—Polysomies or additional chromosomes >2 for centromere of chromosome 10; FIG. 4I—Polysomies (extra copies) of centromeric 3 (epithelial A); FIG. 4J—Polysomies (extra copies) of centromeric 7 (epithelial B); FIG. 4K—Polysomies (extra copies) of centromeric 17 (epithelial C); FIG. 4L—Polysomies (extra copies) or deletions of 9p21.3 (epithelial D); FIG. 4M—Deletions of 3p22.1 in neutrophils, cases vs. controls; FIG. 4N—Polysomies (extra copies) of centromeric 3, in neutrophils cases vs. controls; FIG. 4O—Deletions of 10q22-23 in neutrophils in neutrophils, cases vs. controls; FIG. 4P—Polysomies (extra copies) of centromeric 10 in neutrophils, cases vs. controls; FIG. 4Q—cases versus control, standard deviation of mean area; FIG. 4R—cases versus control, standard deviation of mean integrated optical density; FIG. 4S—cases versus control, standard deviation of mean integrated optical density; FIG. 4T—cases versus control, standard deviation of mean skewness;

FIG. 4U—cases versus control, standard deviation of standard deviation; FIG. 4V—cases versus control, standard deviation of image.

FIGS. 5A-C. Receiver operating characteristic (ROC) curves. FIG. 5A—ROC for model A; FIG. 5B—ROC for model B; FIG. 5C—ROC for model C.

### DETAILED DESCRIPTION OF THE PREFERRED EMBODIMENTS

### I. The Present Invention

The inventors' previous work (U.S. Pat. No. 6,797,471) demonstrated that probes in the 3p22.1 and 10q22 regions were effective at detecting chromosomal abnormalities in patient samples (sputum) that indicated an elevated risk of cancer, predicted cancer progression and metastatic disease. In order to make this assay more specific for lung cancer, and to rule out granulomatous or other diseases such as chromic obstructive airway disease and pulmonary fibrosis, the present inventors have extended this work by developing a new combination of probes (including various centromeric probes) to be used on particular cell types within patient samples. By examining the chromosomal stability of epithelial cells as well as other cells including neutrophilic infiltrate and histiocytes present in patient samples, a highly accurate disease evaluation can be made.

### II. CANCER

Thus, the present invention comprises a method for detecting variation in the hybridization of the probes to DNA to diagnose cancer or as a predictor of cancer development. Such cancers may involve the lung, upper airway primary or secondary cancer, bladder, urithial, head and neck, esophagus, kidney, pancreas, mouth, throat, pharynx, larynx, brain, liver, spleen, small intestine, blood cells, lymph node, colon, breast, endometrium, stomach, prostate, testicle, ovary, skin, bone marrow, blood or other tissue.

In a particular aspect, the present invention relates to the diagnosis and prognosis of smoking related cancers. More particularly, the present invention relates to the diagnosis and prognosis of lung cancer which includes, but is not limited to: squamous cell carcinomas such as squamous carcinoma; small cell carcinomas such as oat cell carcinoma, intermediate cell type carcinoma, combined oat and cell carcinoma; adenocarcinomas such as acinar adenocarcinoma, papillary adenocarcinoma, bronchioloalveolar carcinoma, and solid carcinoma with mucus formation; large cell carcinoma such as giant cell carcinoma and clear cell carcinoma; adenosquamous carcinoma; carcinoid; and bronchial gland carcinomas such as adenoid cystic, and mucoepidermoid carcinoma.

A. Smoking-Related Cancers

The current invention is useful for the prognosis and diagnosis of lung cancers, which can be defined by a number of histologic classifications including: squamous cell carcinomas such as squamous carcinoma; small cell carcinomas such as oat cell carcinoma, intermediate cell type carcinoma, combined oat and cell carcinoma; adenocarcinomas such as acinar adenocarcinoma, papillary adenocarcinoma, bronchioloalveolar carcinoma, and solid carcinoma with mucus formation; large cell carcinoma such as giant cell carcinoma and clear cell carcinoma; adenosquamous carcinoma; carcinoid; and bronchial gland carcinomas such as adenoid cystic, and mucoepidermoid carcinoma. Diagnosis and prognosis of other smoking related cancers is possible with these probes. Squamous cell carcinoma of the head and neck has the same risk factors as lung cancer is hypothesized to have similar etiology (Shriver, 1998). Similarly, smoking is an etiological factor for cancer of the bladder, head, neck, kidneys, pancreas, and cancer of the upper airways including cancer of the mouth, throat, pharynx, larynx, or esophagus.

B. Tumorgenesis

The deletions of various genes in tumor tissue has been well studied in the art. However, there remains a need for probes that are significant for detecting early molecular events in the development of cancers, as well as molecular events that make patients susceptible to the development of cancer. Probes used for the staging of cancer are also of interest. The proposed sequence leading to tumorigenesis includes genetic instability at the cellular or submicroscopic level as demonstrated by loss or gain of chromosomes, leading to a hyperproliferative state due to theoretical acquisition of factors that confer a selective proliferative advantage. Further, at the genetic level, loss of function of cell cycle inhibitors and tumor suppressor genes (TSG), or amplification of oncogenes that drive cell proliferation, are implicated.

Following hyperplasia, a sequence of progressive degrees of dysplasia, carcinoma-in-situ and ultimately tumor invasion is recognized on histology. These histologic changes are both preceded and paralleled by a progressive accumulation of genetic damage. At the chromosomal level genetic instability is manifested by a loss or gain of chromosomes, as well as structural chromosomal changes such as translocation and inversions of chromosomes with evolution of marker chromosomes. In addition cells may undergo polyploidization. Single or multiple clones of neoplastic cells may evolve characterized in many cases by aneuploid cell populations. These can be quantitated by measuring the DNA content or ploidy relative to normal cells of the patient by techniques such as flow cytometry or image analysis.

C. Prognostic Factors and Staging

At present, the most important prognostic factor regarding the survival of patients with lung cancer of non-small cell type is the stage of disease at diagnosis. Small cell cancer usually presents with wide spread dissemination hence the staging system is less applicable. The staging system was devised based on the anatomic extent of cancer and is now know as the TNM system based on anatomical size and spread within the lung and adjacent structures, regional lymph nodes and distant metastases. The only hope presently for a curative procedure lies n the operability of the tumor which can only be resected when the disease is at a low sage, that is confined to the lung.

Occult Carcinoma

| | |
|---|---|
| TX NO MO | Occult carcinoma with bronchopulmonary |
| | secretions containing malignant cells but |
| | without other evidence of the primary tumor or |
| | evidence of metastasis |

Stage 1

| | |
|---|---|
| TIS NO MO | Carcinoma in situ |
| T1 NO MO | Tumor that can be classified T1 without any |
| | metastasis to the regional lymph nodes |
| T1 N1 MO | Tumor that can be classified T1 with metastasis |
| | to the lymph nodes in the ipsilateral hilar |
| | region only |
| T2 N1 MO | Tumor that can be classified T2 without any |
| | metastasis to nodes or distant metastasis |

Stage II

| | |
|---|---|
| T2 N1 MO | Tumor classified as T2 with metastasis to the |
| | lymph nodes in the ipsilateral hilar region only |

Stage III

| | |
|---|---|
| T3 with an N or M | Any tumor more extensive than T2 |
| N2 with an T or M | Any tumor with metastasis to the lymph nodes in |
| | the mediastinum |
| M1 with any T or N | Any tumor with distant metastasis |

D. Grading of Tumors

The histological type and grade of lung cancers do have some prognostic impact within the stage of disease with the best prognosis being reported for stage I adenocarcinoma, with 5 year survival at 50% and 1-year survival at 65% and 59% for the bronchiolar-alveolar and papillary subtypes (Naruke et al., 1988; Travis et al., 1995; Carriaga et al., 1995). For squamous cell carcinoma and large cell carcinoma the 5 year survival is around 35%. Small cell cancer has the worst prognosis with a 5 year survival rate of only 12% for patients with localized disease (Carcy et al., 1980; Hirsh, 1983; Vallmer et al., 1985). For patients with distant metastases survival at 5 years is only 1-2% regardless of histological subtype (Naruke et al., 1988). In addition to histological subtype, it has been shown that histological grading of carcinomas within subtype is of prognostic value with well differentiated tumors having a longer overall survival than poorly differentiated neoplasms. Well differentiated localized adencarcinoma has a 69% overall survival compared to a survival rate of only 34% of patients with poorly differentiated adenocarcinoma (Hirsh, 1983). The 5 year survival rates of patients with localized squamous carcinoma have varied from 37% for well differentiated neoplasms to 25% for poorly differentiated squamous carcinomas (Ihde, 1991).

The histologic criteria for subtyping lung tumors is as follows: squamous cell carcinoma consists of a tumor with keratin formation, keratin pearl formation, and/or intercellular bridges. Adenocarcinomas consist of a tumor with definitive gland formation or mucin production in a solid tumor. Small cell carcinoma consists of a tumor composed of small cells with oval or fusiform nuclei, stippled chromatin, and indistinct nuclei. Large cell undifferentiated carcinoma consists of a tumor composed of large cells with vesicular nuclei and prominent nucleoli with no evidence of squamous or glandular differentiation. Poorly differentiated carcinoma includes tumors containing areas of both squamous and glandular differentiation.

E. Development of Carcinomas

The evolution of carcinoma of the lung is most likely representative of a field cancerization effect as a result of the entire aero-digestive system being subjected to a prolonged period of carcinogenic insults such as benzylpyrenes, asbestosis, air pollution and chemicals other carcinogenic substances in cigarette smoke or other environmental carcinogens. This concept was first proposed by Slaughter et al. (1953). Evidence for existence of a field effect is the common occurrence of multiple synchronous for metachronous second primary tumors (SPTs) that may develop throughout the aero-digestive tract in the oropharynx, upper esophagus or ipsilateral or contralateral lung.

Accompanying these molecular defects is the frequent manifestation of histologically abnormal epithelial changes including hyperplasia, metaplasia, dysplasia, and carcinoma-in-situ. It has been demonstrated in smokers that both the adjacent normal bronchial epithelium as well as the preneoplastic histological lesions may contain clones of genetically altered cells (Wistuba et al., 2000).

Liciardello et al. (1989) found a 10-40% incidence of metachronous tumors and a 9-14% incidence of synchronous SPTs in the upper and lower aero-digestive tract, mostly in patients with the earliest primary tumors SPTs may impose a higher risk than relapse from the original primary tumor and may prove to be the major threat to long term survival following successful therapy for early stage primary head, neck or lung tumors. Hence it is vitally important to follow these patients carefully for evidence of new SPTs in at risk sites for new malignancies specifically in the aero-digestive system.

In addition to chromosomal changes at the microscopic level, multiple blind bronchial biopsies may demonstrate various degrees of intraepithelial neoplasia at loci adjacent to the areas of lung cancer. Other investigators have shown that there are epithelial changes ranging from loss of cilia and basal cell hyperplasia to CIS in most light and heavy smokers and all lungs that have been surgically resected for cancer (Auerbach et al., 1961). Voravud et al. (1993) demonstrated by in-situ hybridization (ISH) studies using chromosome-specific probes for chromosomes 7 and 17 that 30-40% of histologically normal epithelium adjacent to tumor showed polysomies for these chromosomes. In addition there was a progressive increase in frequency of polysomies in the tissue closest to the carcinoma as compared to normal control oral epithelium from patients without evidence of carcinoma. The findings of genotypic abnormalities that increased closer to the area of the tumor support the concept of field cancerization. Interestingly there was no increase in DNA content as measured in the normal appearing mucosa in a Feulgen stained section adjacent to the one where the chromosomes were measured, reflecting perhaps that insufficient DNA had been gained in order to alter the DNA index. Interestingly a very similar increase in DNA content was noted both in dysplastic areas close to the cancer and in the cancerous areas suggesting that complex karyotypic abnormalities that are clonal have already been established in dysplastic epithelium adjacent to lung cancer. Others have also shown an increase in number of cells showing p53 mutations in dysplastic lesions closest to areas of cancer, which are invariably also p53 mutated. Other chromosomal abnormalities that have recently been demonstrated in tumors and dysplastic epithelium of smokers includes deletions of 3p, 17p, 9p and 5q (Feder et al., 1998; Yanagisawa et al., 1996; Thiberville et al., 1995).

F. Chromosome Deletions in Lung Cancer

Small cell lung cancer (SCLC) and non-small cell lung cancer commonly display cytogenetically visible deletions on the short arm of chromosome 3 (Hirano et al., 1994; Valdivieso et al., 1994; Cheon et al, 1993; Pence et al., 1993). This 3p deletion occurs more frequently in the lung tumor tissues of patients who smoke than it does in those of nonsmoking patient. (Rice et al., 1993) Since approximately 85% lung cancer patients were heavy cigarette smokers (Mrkve et al., 1993), 3p might contain specific DNA loci related to the exposure of tobacco carcinogens. It also has been reported that 3p deletion occurs in the early stages of lung carcinogenesis, such as bronchial dysplasia (Pantel et al., 1993). In addition to cytogenetic visible deletions, loss of heterozygosity (LOH) studies have defined 3-21.3 as one of the distinct regions that undergo loss either singly or in combination (Fontanini et al., 1992; Liewald et al., 1992). Several other groups have found large homozygous deletions at 3p21.3 in lung cancer (Macchiarini et al., 1992; Miyamoto et al., 1991; Ichinose et al., 1991; Yamaoka et al., 1990). Transfer of DNA fragments from 3-21.3-3p21.2 into lung tumor cell lines could suppress the tumorigenesis (Sahin et al., 1990; Volm et al., 1989). These finding strongly suggest the presence of at least one tumor suppressor gene in this specific chromosome region whose loss will initiate lung carcinogenesis.

Cytogenetic observation of lung cancer has shown an unusual consistency in the deletion rate of chromosome 3p. In fact, small cell lung cancer (SCLC) demonstrates a 100% deletion rate within certain regions of chromosome 3p. Non small cell lung cancer (NSCLC) demonstrates a 70% deletion rate (Mitsudomi et al., 1996; Shiseki et al., 1996). Loss of heterozygosity and comparative genomic hybridization analysis have shown deletions between 3p14.2 and 3p21.3 to be the most common finding for lung carcinoma and is postulated to be the most crucial change in lung tumorigenesis (Wu et al., 1998). It has been hypothesized that band 3p21.3 is the location for lung cancer tumor suppressor genes. The hypothesis is supported by chromosome 3 transfer studies, which reduced tumorigenicity in lung adenocarcinoma.

Allelotype studies on non-small cell lung carcinoma indicated loss of genetic material on chromosome 10q in 27% of cases. Studies of chromosome 10 allelic loss have shown that there is a very high incidence of LOH in small cell lung cancer, up to 91%. (Alberola et al., 1995; Ayabe et al., 1994). A statistically significant LOH of alleles on 10q was noted in metastatic squamous cell carcinoma (SCC) in 56% of cases compared to non-metastatic SCC with LOH seen in only 14% of cases. (Ayabe et al., 1994). No LOH was seen in other subtypes on NSCLC. Additionally, using micro-satellite polymorphism analysis, it was shown that a high incidence of loss exists between D10s677 and D10S1223. This region spans the long arm of chromosome 10 at bands q21-q24 and overlaps the region deleted in the a study of advanced stage high grade bladder cancers which demonstrated a high frequency of allele loss within a 2.5 cM region at 10q22.3-10q23.1 (Kim et al., 1996).

### III. GENE PROBES

The present invention utilizes probes described from applicant's previous studies, as well as new ones. These probes are described in detail below.

A. 3p22.1 Probes

Structural Features. Recently, the human ribosomal L14 (RPL14) gene (GenBank Accession NM_{—}003973, SEQ ID NO: 1), and the genes CD39L3 (GenBank Accession AAC39884 and AF039917; SEQ ID NO: 3), PMGM (GenBank Accession P15259 and J05073; SEQ ID NO: 5), and GC20 (GenBank Accession NM_{—}005875; SEQ ID NO: 7) were isolated from a BAC (GenBank Accession AC104186, herein incorporated by reference) and located in the 3p22.1 band within the smallest region of deletion overlap of various lung tumors. The RPL14 gene sequence contains a highly polymorphic trinucleotide (CTG) repeat array, which encodes a variable length polyalanine tract. Polyalanine tracts are found in gene products of developmental significance that bind DNA or regulate transcription. For example, Drosophila proteins Engraled, Kruppel and Even-Skipped all contain polyalanine tracts that act as transcriptional repressors. Genotype analysis of RPL14 shows that this locus is 68% heterozygous in the normal population, compared with 25% in NSCLC cell lines. Cell cultures derived from normal bronchial epithelium show a 65% level of heterozygosity, reflecting that of the normal population. See also RP11-391M1/AC104186.

Functional Aspects. Genes with a regulatory function such as the RPL14 gene (SEQ ID NO: 1), along with the genes CD39L3, PMGM, and GC20 (SEQ ID NOS: 3, 5 and 7) and analogs thereof, are good candidates for diagnosis of tumorigenic events. It has been postulated that functional changes of the RPL14 protein (SEQ ID NO: 2) can occur via a DNA deletion mechanism of the trinucleotide repeat encoding for the protein. This deletion mechanism makes the RPL14 gene and attractive sequence that may be used as a marker for the study of lung cancer risk (Shriver et al., 1998). In addition, the RPL14 gene shows significant differences in allele frequency distribution in ethnically defined populations, making this sequence a useful marker for the study of ethnicity adjusting lung cancer (Shriver et al., 1998). Therefore, this gene is useful in the early detection of lung cancer, and in chemopreventive studies as an intermediate biomarker.

B. 10q22 Probes

Structural Features. The 10q22 BAC (46b 12) is 200 Kb and is adjacent and centromeric to PTEN/MMAC1 (GenBank Accession AF067844), which is at 10q22-23 and can be purchased through Research Genetics (Huntsville, Ala.). Alterations to 10q22-25 has been associated with multiple tumors, including lung, prostate, renal, and endomentrial carcinomas, melanoma, and meningiomas, suggesting the possible suppressive locus affecting several cancers in this region. The PTEN/MMAC1 gene, encoding a dual-specificity phosphatase, is located in this region, and has been isolated as a tumor suppressor gene that is altered in several types of human tumors including brain, bladder, breast and prostate cancers. PTEN/MMAC1 mutations have been found in some cancer cell lines, xenografts, and hormone refractory cancer tissue specimens. Because the inventor's 10q22 BAC DNA sequence is adjacent to this region, the DNA sequences in the BAC 10q22 may be involved in the genesis and/or progression of human lung cancer. See also RP11-506M13/AC068139.6

Functional Aspects. Functional evidence for the presence of tumor suppressor genes on 10q has been provided by microcell-mediated chromosomal transfer. The resulting hybrid clones displayed a suppressed tumorigenic phenotype with the inability to proliferate in nude mice and soft agarose. Sequence analysis of the PTEN/MMAC1 gene in lung cancer revealed a G to C substitution located 8 bp upstream of the coding region of exon1 and which seems to be a polymorphism, in 4 of the 30 cases of lung cancer tested. Somatic mutations of the TPEN/MMAC1 gene were not identified in any of the tumors at the primary and metastatic sites of lung cancer, indicating that point mutations in the PTEN/MMAC1 gene are probably not an important factor in tumorigenesis and the progression of a major subset of lung cancers. Other more important tumor suppressor genes must lie close to the PTEN/MMAC1 gene, in the vicinity of the inventors' 10q22 BAC locus. Therefor, the 10q22 probe is useful in the further development of clinical biomarkers for the early detection of neoplastic events, for risk assessment and monitoring the efficacy of chemoprevention therapy in high risk former or current smokers.

C. 1q21 Probes

Structural Features. Chromosome 1q21 overrepresentation is the most frequent imbalance in a variety of human solid tumors, especially in adenocarcinomas of other origins and it is associated with adenoid differentiation in the lung. However, the gain of 1q21 is also correlated with tumor dissemination, for example, in lung SCC, renal clear cell carcinomas, and brain metastasis formation, suggesting that the centromeric region 1q21 is particularly important. Recently, amplification was suggested to be one of major causes of the higher propensity of lung adenocarcinoma for haematogeneous dissemination compared with lung SCC, implying that aberration of the genes located in the hot region may play key role in the prognosis of patients who have lung cancer. The 1q21 probe utilized by the inventors for this study derives from the RP11-49N14 open reading frame (mRNA at Accesssion No. NM_{—}005978).

Functional Aspects. The inventors recently identified a candidate gene encoding the human S100A2, a calcium-binding protein. S100A2 is an important member of the S100 family, which includes a group of small acidic proteins with common EF-hand calcium-binding motifs. S100 proteins regulate a variety of cellular processes, including cellular proliferation, differentiation, motility, secretion, membrane permeability, protein synthesis and extracellular signal transduction. At least 13 different S100 genes have been found in a clustered organization on human chromosome 1q21, whose frequent rearrangement in human neoplasms possibly influences the altered expression of some S100 proteins in tumor cells. Recently, S100A2 was found to be upregulated in melanomas and was suggested to be a useful marker of epithelial cells in the different skin tumors. In a published study, the inventors investigated the prognostic significance of S100A2 expression in the early-stage non small lung cancer (NSCLC). Immunohistochemical analysis to determine the percentage of cells staining positive for S100A2 was performed on 11 NSCLC tissue microarray slides containing samples from 113 patients with pathologic stage I NSCLC who had undergone curative surgery. S100A2 was expressed in samples from 79 patients (69.9%). Kaplan-Meier analysis showed that patients whose tumors had positive S100A2 expression had a significantly lower overall survival and disease-specific survival rate at 5 years after surgery than did patients with negative S100A2 expression (<0.001 and p<0.001, respectively). Age at diagnosis, histologic type of cancer, degree of differentiation and smoking history did not have a statistically significant effect on survival. Multivariate analysis confirmed that S100A2 expression is a better predictor for disease-specific survival than were other clinical and histologic variables tested. These results suggested that the expression of the S100A2 protein in stage I NSCLC indicates poor prognosis and may be used to identify patients with early-stage NSCLC who might benefit from adjuvant treatment.

D. The Cen17 Probes

Structural Features. The Cen17 probe was designed from alpha satellite DNA in the centremeric region of chromosome 17. The probe is used to examine numerical changes of chromosome 17 in human tumors because the chromosome contains two classified well know tumor-specific genes, p53 and HER2 on 17p13.1 and 17q21, respectively. The structure of p53 appears to be unique, consisting of a large beta-sandwich that acts as a scaffold for 3 loop-based elements. The open reading frame of p53 is 393 amino acids long, with the central region containing the DNA-binding domain. This proteolysis-resistant core is flanked by a C-terminal end mediating oligomerization and an N-terminal end containing a strong transcription activation signal. succeeded in co-crystallizing the core domain of p53 bound to DNA. The sandwich is composed of two anti-parallel α-sheets containing 4 and 5 β-strands, respectively. The first loop binds to DNA within the major groove, the second loop binds to DNA within the minor groove, and the third loop packs against the second loop to stabilize it. The residues most frequently mutated in cancers are all at or near the protein-DNA interface, and over two-thirds of the missense mutations are in 1 of the 3 DNA loops. Structurally, HER2 complexes with the Herceptin antigen-binding fragment (Fab) at 2.5 angstroms. These structures revealed a fixed conformation for HER2 that resembles a ligand-activated state, and showed HER2 poised to interact with other ErbB receptors in the absence of direct ligand binding. Herceptin binds to the juxtamembrane region of HER2, identifying this site as a target for anticancer therapies.

Functional Aspects. Using the Cen17 probe in FISH detection of cancer cells from patients who have lung cancer is very important. For example, conventionally, flow cytometry is common tool to determine ploidy in tissue from lung carcinomas. However, the DNA-ploidy pattern produced by flow cytometry is representative of the entire DNA content of the tissue being examined. Therefore, a small population of abnormal cells may be difficult to detect within a much larger population of normal cells. The selection of tumor cells and the elimination of normal cells from the evaluation, potentially enhances the sensitivity of detection of DNA-content abnormalities when the proportion of tumor cells is small. When FISH with Cen 17 probe was compared with flow cytometry in terms of ability to determine ploidy in tissue from lung carcinomas, FISH is a more sensitive method of ploidy analysis than flow cytometry. Functionally, p53 is postulated to bind as a tetramer to a p53-binding site (PBS) and to activate the expression of adjacent genes that inhibit growth and/or invasion. Deletion of one or both p53 alleles reduces the expression of tetramers, resulting in decreased expression of the growth inhibitory genes.

The p53 tumor antigen is found in increased amounts in a wide variety of transformed cells and tumor cells including lung cancer. In lung cancer, an insertion/duplication of GCATACGTGATG at nucleotide 2322 of the ERBB2 gene may result in a 4-amino acid insertion (AYVM) at codon 774. This mutation was not identified in DNA from normal tissue. The same insertion was found in tumor tissue only from another individual, and also in tumor tissue from a third individual from whom no normal tissue was available for comparison. Furthermore, HER2 gene copy numbers per cell were evaluated by fluorescent in situ hybridization (FISH) in 102 NSCLC patients treated with gefitinib, and previously evaluated for EGFR status by FISH, immunohistochemistry, and presence of mutations. Patients with HER2 high copy number (high polysomy and gene amplification—HER2 FISH-positive) represented 22.8% of patients, and compared with patients with no or low gain (HER2 FISH-negative), had significantly better objective response (OR, 34.8% v 6.4%; P=0.001), disease control rate (DCR, 56.5% v 33.3%; P=0.04), time to progression (TTP, 9.05 v 2.7 months; P=0.02), and a trend toward longer overall survival (OS, 20.8 versus 8.4 months; P=0.056). HER2 protein expression investigated by immunohistochemistry was positive in only five of 72 (7%) patients analyzed and all 89 patients tested by DNA sequencing were negative for mutations in HER2 exon 20. Patients with HER2 FISH-positive tumors displaying increased expression of EGFR protein, gene gain, or mutations (EGFR-positive) had a significantly better OR, DCR, TTP, and OS than patients negative for both receptors. Increased copy number of the HER2 gene is associated with gefitinib sensitivity in EGFR-positive patients, supporting use of HER2 FISH analysis for selection of patients for TKI therapy.

E. 9p21.3

Structural Features. Chromosome region 9p21 is involved in chromosomal inversions, translocations, heterozygous deletions, and homozygous deletions in a variety of malignant cell lines including those from glioma, non-small cell lung cancer, leukemia, and melanoma. Deletion of 9p21 markers is found in more than half of all melanoma cell lines. These findings suggest that 9p21 contains a tumor suppressor locus that may be involved in the genesis of several tumor types. Kamb et al. (1996) localized a putative tumor suppressor locus to band 9p21 in a region of less than 40 kb by means of analyzing homozygous deletions in melanoma cell lines. The region was found to contain a gene, called p16 gene (CDKN2A), which was homozygously deleted at high frequency in cell lines derived from tumors of lung, breast, brain, bone, skin, bladder, kidney, ovary, and lymphocyte. Melanoma cell lines carried at least one copy of p 16 in combination with a deleted allele. Melanoma cell lines that carried at least 1 copy of p16 frequently showed nonsense, missense, or frameshift mutations in the gene. Thus, p16 may rival p53 in the universality of its involvement in tumorigenesis.

Functional Aspects. The frequent deletion or mutation of p16 in tumor cells suggests that p16 acts as a tumor suppressor. Lukas et al. (1995) showed that wildtype p16 arrests normal diploid cells in late GI, whereas a tumor-associated mutant of p16 does not. Significantly, the ability of p16 to induce cell cycle arrest was lost in cells lacking functional retinoblastoma protein (RB1). Thus, loss of p16, overexpression of D-cyclins, and loss of retinoblastoma have similar effects on GI progression, and may represent a common pathway to tumorigenesis. The mutation was a C-to-T transition changing proline −114 to leucine and had been observed in three independent melanoma cell lines. p16 can act as a potent and specific inhibitor of progression through the GI phase of the cell cycle and that several tumor-derived alleles of p16 encode functionally compromised proteins. In vivo, the presence of functional retinoblastoma protein appeared to be necessary but perhaps not sufficient to confer full sensitivity to p16-mediated growth arrest. In lung cancer, loss of heterozygosity of 9p21 was detected in all of the 23 tumors and homozygous deletions of the p16/CDKN2 locus were detected in 6 of the 23 (26%) tumors, suggesting FISH analysis with probes containing p16 locus could be used as molecular markers to assay sputum samples for premalignant cells exfoliated from the bronchial epithelium.

F. Cen1, Cen3, Cen9 and Cen10 Probes

Cen1 is a centromeic probe for chromosome 1, which is the largest chromosome. Thus, usually Cen1 is used as internal control probe for testing numeric aberrations of the genes in the same chromosome or probes located on other probes. For example, to examine changes of 5p15, 8q24 (site of the c-myc gene), and 7p12 (site of the EGFR gene) loci in lung cancer, Cen1 was used in combination with the probes specific for the three loci in a FISH assay. The assay was performed on 74 specimens that had been assessed previously for evidence of malignancy by routine cytology with Pap staining. Forty-eight patients had histologically confirmed lung carcinoma and 26 patients had a clinical diagnosis that was negative for lung carcinoma. FISH analysis was performed without knowledge of the patient's clinical information. The finding of six or more epithelial cells with gains of two or more chromosome regions was considered a positive FISH result (i.e., evidence of malignancy). The sensitivity of FISH for the detection of lung carcinoma was 82% in this set of specimens compared with a 54% sensitivity by design for cytology (FISH vs. cytology, p=0.007). FISH detected 15 of 18 specimens that were falsely negative by cytology. The specificities of FISH and cytology were 82% and 100%, respectively, and were not significantly different (p=0.993). The data indicate a potential utility of the FISH assay as an adjunct to bronchial washing cytology in routine clinical practice. In addition, structural rearrangements in lung cancer were most frequently observed in chromosome 1 itself.

Cen3 is a centromeic probe for chromosome 3. Hemizygous deletion in the chromosome 3 is a common finding in non-small cell lung carcinoma and is postulated to be a crucial early change in lung tumorigenesis. Most importantly, the chromosome harbors several key tumor suppressor genes, which are targets for developing diagnostic biomarker for lung cancer. For example, thirty-seven biopsy specimens from 13 patients were examined for loss of 3p14 by FISH. Deletion of 3p was found in all the tumors. In contrast, no alterations were detected for the same regions in the nine patients without primary lung cancer. Furthermore, the inventors' previous study using 3p22 probe demonstrated the region contains tumor suppressor genes and the DNA probe testing the region might be powerful tool for the early detection of lung cancer.

Cen9 is a centromeic probe for chromosome 9. Genomic regions in the chromosome, for example, 9p21, are involved in chromosomal inversions, translocations, heterozygous deletions, and homozygous deletions in a variety of malignant cell lines including those from glioma, nonsmall cell lung cancer, leukemia, and melanoma. Therefore, Cen9 can be used as internal control for testing copy aberrations of the gene in chromosome 9. for example. Fluorescence in situ hybridization analysis using Cen9 and genomic probes spanning either the p16 or Hel-N1 (located at D9S126) gene was performed in 14 lung cancer. The results from FISH suggested that the p16 region is the major target of deletion at chromosome 9p21 in primary NSCLC.

Cen10 is a centromeic probe for chromosome 10. Numerical changes of chromosome are one of the most common genomic aberrations in human solid tumors including lung cancer. Therefore, Cen10 is not only used for examining chromosome 10 aberrations, but also as control to test abnormalities of other genes in the in the same chromosome. To determine whether SP-A aberrations are lung cancer-specific and indicate smoking-related damage, the inventors used tricolor fluorescence in situ hybridization with SP-A and PTEN probes on the lung tumors from 28 patients with primary NSCLC. Both of SP-A and PTEN locate on 10q arm. To further define the clinical relevance of SP-A aberrations, fluorescence in situ hybridization was performed on both tumor cells and adjacent bronchial tissue cells from paraffin-embedded tissue blocks from 130 patients NSCLC for whom we had follow-up information. SP-A was deleted from 89% of cancer tissues and the deletion was related to the smoking status of patients (P<0.001). PTEN was deleted from 16% in the cancer tissues and the deletion was not related to the smoking status of patients (P>0.05). In the cells isolated from paraffin-embedded tissue blocks, SP-A was deleted from 87% of the carcinoma tissues and 32% of the adjacent normal-appearing bronchial tissues. SP-A deletions in tumors and adjacent normal-appearing bronchial tissues were associated with increases in the risk of disease relapse (P=0.0035 and P<0.001, respectively). SP-A deletions in the bronchial epithelium were the strongest prognostic indicators of disease-specific survival (P=0.025). Deletions of the SP-A gene are specific genomic aberrations in bronchial epithelial cells adjacent to and within NSCLC, and are associated with tumor progression and a history of smoking. SP-A deletions might be a useful biomarker to identify poor prognoses in patients with NSCLC who might therefore benefit from adjuvant treatment.

G. FDPS, BCL7A and MMP-24

Using microarrays, the inventors analysed the DNA from 12 sputa samples comprising sputa from pooled normal controls (3), pooled adenocarcinomas (3), pooled squamous carcinomsa (3) and pooled sputa from patients with granulomas (3). No attempt was made to separate out components such as epithelial cells, neutrophils, granulocytes or macrophages prior to extraction.

The inventors discovered three new DNA probes that had their lowest expression in normal patients, followed by higher levels in granulomas, with highest expression or amplification noted in either adenocarcinoma or squamous carcinoma. These probes corresponding to regions of gene amplification related to regions from FDPS (farnesyl diphophate synthase), BCL7A and MMP24. These genes have not, to the best of the inventors' knowledge, been previously described to be amplified in non-small cell lung cancer. NM_{—}173639, NM_{—}006690 and NM_{—}020993 refer to the corresponding messenger RNA sequences.

Farnesyl diphosphate synthase (FDPS) is a key enzyme in isoprenoid biosynthesis which supplies sesquiterpene precursors for several classes of essential metabolites including sterols, dolichols, ubiquinones and carotenoids as well as substrates for farnesylation and geranylgeranylation of proteins. It catalyzes the sequential head-to-tail condensation of two molecules of isopentenyl diphosphate with dimethylallyl diphosphate. The enzyme is a homodimer of subunits, typically having two aspartate-rich motifs with two sets of substrate binding sites for an allylic diphosphate and isopentenyl diphosphate per homodimer. The synthase amino-acid residues at the 4th and 5th positions before the first aspartate rich motif mainly determine product specificity. Hypothetically, type I (eukaryotic) and type II (eubacterial) FPPSs evolved from archeal geranylgeranyl diphosphate synthase by substitutions in the chain length determination region. FPPS belongs to enzymes encoded by gene families. The gene or farnesyl diphosphate synthase is located on chromosome 1p31.1.

The gene for BCL7A is located at 12q24.11 and its molecular function has to do with Actin binding. This protein, designated BCL7A, exhibits no recognizable protein motifs but shows homology with the actin-binding protein, caldesmon. In the Burkitt lymphoma cell line Wein 133, containing the 3-way translocation, the BCL7A breakpoint occurred within the first intron and resulted in a free-of-fusion transcript between MYC and BCL7A, with exon 1 of BCL7A being replaced by MYC exon 1. The normal, untranslocated allele of BCL7A was also expressed without mutation. One of the 11 other BNCLs with 12q24.1 cytogenetic abnormalities showed biallelic rearrangement within the first intron of BCL7A, which was adjacent to the breakpoint observed in Wein 133. Zani et al. (1996) concluded that disruption of the amino-terminus of BCL7A defined a new mechanism in the pathogenesis of a subset of high-grade BNHL.

The matrix metalloproteinases (MMPs) are a family of zinc-dependent endopeptidases that degrade the different protein components of the extracellular matrix and basement membranes. Based on their primary structures, substrate specificity, and cellular localization, the human MMPs can be classified into at least 4 main subfamilies: the collagenases, gelatinases, stromelysins, and membrane-type MMPs (MT-MMPs). By searching an EST database for sequences similar to previously described MMPs, Llano et al. (1999) identified MMP24, which they called MT5-MMP. They isolated a complete MMP24 coding sequence. The predicted 645-amino acid MMP24 protein displays a number of features characteristic of MMPs, including a signal sequence, a prodomain with a cysteine residue essential for maintaining latency, a catalytic domain of approximately 170 residues that contains a consensus sequence involved in zinc binding, and an approximately 200-amino acid segment with sequence similarity to hemopexin (142290). In addition, MMP24 contains 3 insertions characteristic of the MT subclass of MMPs, including a C-terminal extension consisting of putative transmembrane and cytoplasmic domains. The authors demonstrated that recombinant MMP24 protein expressed in mammalian cells localizes to the plasma membrane. They showed that the catalytic domain of MMP24 exhibits potent proteolytic activity against progelatinase A (MMP2; 120360). Northern blot analysis of normal human tissues detected an approximately 4.5-kb MMP24 transcript predominantly in brain and slightly smaller MMP24 transcripts in kidney, pancreas, and lung. Analysis of MMP24 expression in a variety of brain tumors, including astrocytomas, anaplastic astrocytomas, glioblastomas, mixed gliomas, oligodendrogliomas, ependymomas, neurocytomas, and meningiomas, indicated that MMP24 was significantly overexpressed in several astrocytomas, anaplastic astrocytomas, and glioblastomas, as compared to normal brain tissue. In contrast, MMP24 was expressed at very low or undetectable levels in all meningiomas tested. These data suggest that MT5-MMP may contribute to the activation of progelatinase A in tumor tissues, in which it is overexpressed, thereby facilitating tumor progression.

By FISH, Llano et al. (1999) mapped the MMP24 gene to 20q11.2. By radiation hybrid analysis and FISH, Kinoh et al. (1999) mapped the human MMP24 gene to 20q11.2-q12 and the mouse homolog to chromosome 2 fluorescent in situ hybridization experiments showed that the human MT5-MMP gene (MMP-24) maps to 20q11.2, a region frequently amplified in tumors from diverse sources including lung cancer.

### IV. METHODS FOR ASSESSING GENE STRUCTURE

In accordance with the present invention, one will utilize various probes to examine the structure of genomic DNA from patient samples. A wide variety of methods may be employed to detect changes in the structure of various chromosomal regions. The following is a non-limiting discussion of such methods.

A. Fluorescence In Situ Hybridization

Fluorescence in situ hybridization (FISH) can be used for molecular studies. FISH is used to detect highly specific DNA probes which have been hybridized to chromosomes using fluorescence microscopy. The DNA probe is labeled with fluorescent or non fluorescent molecules which are then detected by fluorescent antibodies. The probes bind to a specific region or regions on the target chromosome. The chromosomes are then stained using a contrasting color, and the cells are viewed using a fluorescence microscope.

Each FISH probe is specific to one region of a chromosome, and is labeled with fluorescent molecules throughout it's length. Each microscope slide contains many metaphases. Each metaphase consists of the complete set of chromosomes, one small segment of which each probe will seek out and bind itself to. The metaphase spread is useful to visualize specific chromosomes and the exact region to which the probe binds. The first step is to break apart (denature) the double strands of DNA in both the probe DNA and the chromosome DNA so they can bind to each other. This is done by heating the DNA in a solution of formamide at a high temperature (70-75° C.) Next, the probe is placed on the slide and the slide is placed in a 37° C. incubator overnight for the probe to hybridize with the target chromosome. Overnight, the probe DNA seeks out it's target sequence on the specific chromosome and binds to it. The strands then slowly reanneal. The slide is washed in a salt/detergent solution to remove any of the probe that did not bind to chromosomes and differently colored fluorescent dye is added to the slide to stain all of the chromosomes so that they may then be viewed using a fluorescent light microscope. Two, or more different probes labeled with different fluorescent tags can be mixed and used at the same time. The chromosomes are then stained with a third color for contrast. This gives a metaphase or interphase cell with three or more colors which can be used to detect different chromosomes at the same time, or to provide a control probe in case one of the other target sequences are deleted and a probe cannot bind to the chromosome. This technique allows, for example, the localization of genes and also the direct morphological detection of genetic defects.

The advantage of using FISH probes over microsatellite instability to test for loss of allelic heterozygosity is that the (a) FISH is easily and rapidly performed on cells of interest and can be used on paraffin-embedded, or fresh or frozen tissue allowing the use of micro-dissection (b) specific gene changes can be analyzed on a cell by cell basis in relationship to centomeric probes so that true homozygosity versus heterozygosity of a DNA sequence can be evaluated (use of PCR™ for microsatellite instability may permit amplification of surrounding normal DNA sequences from contamination by normal cells in a homozygously deleted region imparting a false positive impression that the allele of interest is not deleted) (c) PCR cannot identify amplification of genes d) FISH using bacterial artificial chromosomes (BACs) permits easy detection and localization on specific chromosomes of genes of interest which have been isolated using specific primer pairs.

B. Template Dependent Amplification Methods

A number of template dependent processes are available to amplify the marker sequences present in a given template sample. One of the best known amplification methods is the polymerase chain reaction (referred to as PCR™) which is described in detail in U.S. Pat. Nos. 4,683,195, 4,683,202 and 4,800,159, and in Innis et al., 1990, each of which is incorporated herein by reference in its entirety.

Briefly, in PCR™, two primer sequences are prepared that are complementary to regions on opposite complementary strands of the marker sequence. An excess of deoxynucleoside triphosphates are added to a reaction mixture along with a DNA polymerase, e.g., Taq polymerase. If the marker sequence is present in a sample, the primers will bind to the marker and the polymerase will cause the primers to be extended along the marker sequence by adding on nucleotides. By raising and lowering the temperature of the reaction mixture, the extended primers will dissociate from the marker to form reaction products, excess primers will bind to the marker and to the reaction products and the process is repeated.

A reverse transcriptase PCR™ amplification procedure may be performed in order to quantify the amount of mRNA amplified. Methods of reverse transcribing RNA into cDNA are well known and described in Sambrook et al. (1989). Alternative methods for reverse transcription utilize thermostable, RNA-dependent DNA polymerases. These methods are described in WO 90/07641 filed Dec. 21, 1990. Polymerase chain reaction methodologies are well known in the art.

Another method for amplification is the ligase chain reaction (“LCR”), disclosed in EPO No. 320 308, incorporated herein by reference in its entirety. In LCR, two complementary probe pairs are prepared, and in the presence of the target sequence, each pair will bind to opposite complementary strands of the target such that they abut. In the presence of a ligase, the two probe pairs will link to form a single unit. By temperature cycling, as in PCR™, bound ligated units dissociate from the target and then serve as “target sequences” for ligation of excess probe pairs. U.S. Pat. No. 4,883,750 describes a method similar to LCR for binding probe pairs to a target sequence.

Qbeta Replicase, described in PCT Application No. PCT/US87/00880, may also be used as still another amplification method in the present invention. In this method, a replicative sequence of RNA that has a region complementary to that of a target is added to a sample in the presence of an RNA polymerase. The polymerase will copy the replicative sequence that can then be detected.

An isothermal amplification method, in which restriction endonucleases and ligases are used to achieve the amplification of target molecules that contain nucleotide 5′-[alpha-thio]-triphosphates in one strand of a restriction site may also be useful in the amplification of nucleic acids in the present invention (Walker et al., 1992).

Strand Displacement Amplification (SDA) is another method of carrying out isothermal amplification of nucleic acids, which involves multiple rounds of strand displacement and synthesis, i.e., nick translation. A similar method, called Repair Chain Reaction (RCR), involves annealing several probes throughout a region targeted for amplification, followed by a repair reaction in which only two of the four bases are present. The other two bases can be added as biotinylated derivatives for easy detection. A similar approach is used in SDA. Target specific sequences can also be detected using a cyclic probe reaction (CPR). In CPR, a probe having 3′ and 5′ sequences of non-specific DNA and a middle sequence of specific RNA is hybridized to DNA that is present in a sample. Upon hybridization, the reaction is treated with RNase H, and the products of the probe identified as distinctive products that are released after digestion. The original template is annealed to another cycling probe and the reaction is repeated.

Still another amplification methods described in GB Application No. 2 202 328, and in PCT Application No. PCT/US89/01025, each of which is incorporated herein by reference in its entirety, may be used in accordance with the present invention. In the former application, “modified” primers are used in a PCR-like, template- and enzyme-dependent synthesis. The primers may be modified by labeling with a capture moiety (e.g., biotin) and/or a detector moiety (e.g., enzyme). In the latter application, an excess of labeled probes are added to a sample. In the presence of the target sequence, the probe binds and is cleaved catalytically. After cleavage, the target sequence is released intact to be bound by excess probe. Cleavage of the labeled probe signals the presence of the target sequence.

Other nucleic acid amplification procedures include transcription-based amplification systems (TAS), including nucleic acid sequence based amplification (NASBA) and 3SR (Kwoh et al., 1989; Gingeras et al., PCT Application WO 88/10315, incorporated herein by reference in their entirety). In NASBA, the nucleic acids can be prepared for amplification by standard phenol/chloroform extraction, heat denaturation of a clinical sample, treatment with lysis buffer and minispin columns for isolation of DNA and RNA or guanidinium chloride extraction of RNA. These amplification techniques involve annealing a primer which has target specific sequences. Following polymerization, DNA/RNA hybrids are digested with RNase H while double stranded DNA molecules are heat denatured again. In either case the single stranded DNA is made fully double stranded by addition of second target specific primer, followed by polymerization. The double-stranded DNA molecules are then multiply transcribed by an RNA polymerase such as T7 or SP6. In an isothermal cyclic reaction, the RNA's are reverse transcribed into single stranded DNA, which is then converted to double stranded DNA, and then transcribed once again with an RNA polymerase such as T7 or SP6. The resulting products, whether truncated or complete, indicate target specific sequences.

Davey et al., EPO No. 329 822 (incorporated herein by reference in its entirety) disclose a nucleic acid amplification process involving cyclically synthesizing single-stranded RNA (“ssRNA”), ssDNA, and double-stranded DNA (dsDNA), which may be used in accordance with the present invention. The ssRNA is a template for a first primer oligonucleotide, which is elongated by reverse transcriptase (RNA-dependent DNA polymerase). The RNA is then removed from the resulting DNA:RNA duplex by the action of ribonuclease H(RNase H, an RNase specific for RNA in duplex with either DNA or RNA). The resultant ssDNA is a template for a second primer, which also includes the sequences of an RNA polymerase promoter (exemplified by T7 RNA polymerase) 5′ to its homology to the template. This primer is then extended by DNA polymerase (exemplified by the large “Klenow” fragment of E. coli DNA polymerase I), resulting in a double-stranded DNA (“dsDNA”) molecule, having a sequence identical to that of the original RNA between the primers and having additionally, at one end, a promoter sequence. This promoter sequence can be used by the appropriate RNA polymerase to make many RNA copies of the DNA. These copies can then re-enter the cycle leading to very swift amplification. With proper choice of enzymes, this amplification can be done isothermally without addition of enzymes at each cycle. Because of the cyclical nature of this process, the starting sequence can be chosen to be in the form of either DNA or RNA.

Miller et al., PCT Application WO 89/06700 (incorporated herein by reference in its entirety) disclose a nucleic acid sequence amplification scheme based on the hybridization of a promoter/primer sequence to a target single-stranded DNA (“ssDNA”) followed by transcription of many RNA copies of the sequence. This scheme is not cyclic, i.e., new templates are not produced from the resultant RNA transcripts. Other amplification methods include “RACE” and “one-sided PCR” (Frohman, 1990; Ohara et al., 1989; each herein incorporated by reference in their entirety).

Methods based on ligation of two (or more) oligonucleotides in the presence of nucleic acid having the sequence of the resulting “di-oligonucleotide,” thereby amplifying the di-oligonucleotide, may also be used in the amplification step of the present invention (Wu et al., 1989, incorporated herein by reference in its entirety).

C. Southern/Northern Blotting

Blotting techniques are well known to those of skill in the art. Southern blotting involves the use of DNA as a target, whereas Northern blotting involves the use of RNA as a target. Each provide different types of information, although cDNA blotting is analogous, in many aspects, to blotting or RNA species.

Briefly, a probe is used to target a DNA or RNA species that has been immobilized on a suitable matrix, often a filter of nitrocellulose. The different species should be spatially separated to facilitate analysis. This often is accomplished by gel electrophoresis of nucleic acid species followed by “blotting” on to the filter.

Subsequently, the blotted target is incubated with a probe (usually labeled) under conditions that promote denaturation and rehybridization. Because the probe is designed to base pair with the target, the probe will binding a portion of the target sequence under renaturing conditions. Unbound probe is then removed, and detection is accomplished as described above.

D. Separation Methods

It normally is desirable, at one stage or another, to separate the amplification product from the template and the excess primer for the purpose of determining whether specific amplification has occurred. In one embodiment, amplification products are separated by agarose, agarose-acrylamide or polyacrylamide gel electrophoresis using standard methods. See Sambrook et al., 1989.

Alternatively, chromatographic techniques may be employed to effect separation. There are many kinds of chromatography which may be used in the present invention: adsorption, partition, ion-exchange and molecular sieve, and many specialized techniques for using them including column, paper, thin-layer and gas chromatography (Freifelder, 1982).

E. Detection Methods

Products may be visualized in order to confirm amplification of the marker sequences. One typical visualization method involves staining of a gel with ethidium bromide and visualization under UV light. Alternatively, if the amplification products are integrally labeled with radio- or fluorometrically-labeled nucleotides, the amplification products can then be exposed to x-ray film or visualized under the appropriate stimulating spectra, following separation.

In one embodiment, visualization is achieved indirectly. Following separation of amplification products, a labeled nucleic acid probe is brought into contact with the amplified marker sequence. The probe preferably is conjugated to a chromophore but may be radiolabeled. In another embodiment, the probe is conjugated to a binding partner, such as an antibody or biotin, and the other member of the binding pair carries a detectable moiety.

In one embodiment, detection is by a labeled probe. The techniques involved are well known to those of skill in the art and can be found in many standard books on molecular protocols. See Sambrook et al. (1989). For example, chromophore or radiolabel probes or primers identify the target during or following amplification.

One example of the foregoing is described in U.S. Pat. No. 5,279,721, incorporated by reference herein, which discloses an apparatus and method for the automated electrophoresis and transfer of nucleic acids. The apparatus permits electrophoresis and blotting without external manipulation of the gel and is ideally suited to carrying out methods according to the present invention.

In addition, the amplification products described above may be subjected to sequence analysis to identify specific kinds of variations using standard sequence analysis techniques. Within certain methods, exhaustive analysis of genes is carried out by sequence analysis using primer sets designed for optimal sequencing (Pignon et al, 1994). The present invention provides methods by which any or all of these types of analyses may be used.

F. Kit Components

All the essential materials and reagents required for detecting changes in the chromosomal regions discussed above may be assembled together in a kit. This generally will comprise preselected primers and probes. Also included may be enzymes suitable for amplifying nucleic acids including various polymerases (RT, Taq, Sequenase™, etc.), deoxynucleotides and buffers to provide the necessary reaction mixture for amplification, and optionally labeling agents such as those used in FISH. Such kits also generally will comprise, in suitable means, distinct containers for each individual reagent and enzyme as well as for each primer or probe.

G. Chip Technologies

Specifically contemplated by the present inventors are chip-based DNA technologies such as those described by Hacia et al. (1996) and Shoemaker et al. (1996). These techniques involve quantitative methods for analyzing large numbers of genes rapidly and accurately. By tagging genes with oligonucleotides or using fixed probe arrays, one can employ chip technology to segregate target molecules as high density arrays and screen these molecules using methods such as fluorescence, conductance, mass spectrometry, radiolabeling, optical scanning, or electrophoresis. See also Pease et al. (1994); Fodor et al. (1991).

Biologically active DNA probes may be directly or indirectly immobilized onto a surface to ensure optimal contact and maximum detection. When immobilized onto a substrate, the gene probes are stabilized and therefore may be used repetitively. In general terms, hybridization is performed on an immobilized nucleic acid target or a probe molecule is attached to a solid surface such as nitrocellulose, nylon membrane or glass. Numerous other matrix materials may be used, including reinforced nitrocellulose membrane, activated quartz, activated glass, polyvinylidene difluoride (PVDF) membrane, polystyrene substrates, polyacrylamide-based substrate, other polymers such as poly(vinyl chloride), poly(methyl methacrylate), poly(dimethyl siloxane), photopolymers (which contain photoreactive species such as nitrenes, carbenes and ketyl radicals capable of forming covalent links with target molecules (Saiki et al., 1994).

Immobilization of the gene probes may be achieved by a variety of methods involving either non-covalent or covalent interactions between the immobilized DNA comprising an anchorable moiety and an anchor. DNA is commonly bound to glass by first silanizing the glass surface, then activating with carbodimide or glutaraldehyde. Alternative procedures may use reagents such as 3-glycidoxypropyltrimethoxysilane (GOP) or aminopropyltrimethoxysilane (APTS) with DNA linked via amino linkers incorporated either at the 3′ or 5′ end of the molecule during DNA synthesis. Gene probe may be bound directly to membranes using ultraviolet radiation. With nitrocellous membranes, the probes are spotted onto the membranes. A UV light source is used to irradiate the spots and induce cross-linking. An alternative method for cross-linking involves baking the spotted membranes at 80° C. for two hours in vacuum.

Immobilization can consist of the non-covalent coating of a solid phase with streptavidin or avidin and the subsequent immobilization of a biotinylated polynucleotide (Holmstrom, 1993). Precoating a polystyrene or glass solid phase with poly-L-Lys or poly L-Lys, Phe, followed by the covalent attachment of either amino- or sulfliydryl-modified polynucleotides using bifunctional crosslinking reagents (Running, 1990 and Newton, 1993) can also be used to immobilize the probe onto a surface.

Immobilization may also take place by the direct covalent attachment of short, 5′-phosphorylated primers to chemically modified polystyrene plates (“Covalink” plates, Nunc) Rasmussen, (1991). The covalent bond between the modified oligonucleotide and the solid phase surface is introduced by condensation with a water-soluble carbodiimide. This method facilitates a predominantly 5′-attachment of the oligonucleotides via their 5′-phosphates.

Nikiforov et al. (U.S. Pat. No. 5,610,287) describes a method of non-covalently immobilizing nucleic acid molecules in the presence of a salt or cationic detergent on a hydrophilic polystyrene solid support containing an —OH, —C═O or —COOH hydrophilic group or on a glass solid support. The support is contacted with a solution having a pH of about 6 to about 8 containing the synthetic nucleic acid and the cationic detergent or salt. The support containing the immobilized nucleic acid may be washed with an aqueous solution containing a non-ionic detergent without removing the attached molecules.

There are two common variants of chip-based DNA technologies involving DNA microarrays with known sequence identity. For one, a probe cDNA (500-5,000 bases long) is immobilized to a solid surface such as glass using robot spotting and exposed to a set of targets either separately or in a mixture. This method, “traditionally” called DNA microarray, is widely considered as developed at Stanford University. A recent article by Ekins and Chu (1999) provides some relevant details. The other variant includes an array of oligonucleotide (20˜25-mer oligos) or peptide nucleic acid (PNA) probes is synthesized either in situ (on-chip) or by conventional synthesis followed by on-chip immobilization. The array is exposed to labeled sample DNA, hybridized, and the identity/abundance of complementary sequences are determined. This method, “historically” called DNA chips, was developed at Affymetrix, Inc., which sells its products under the GeneChip® trademark.

### V. NUCLEIC ACIDS

The inventors' provide probes for various human chromosomal regions as discussed above. However, the present invention is not limited to the use of the specific nucleic acid segments disclosed herein. Rather, a variety of alternative probes that target the same regions/polymorphisms may be employed.

A. Probes and Primers

Naturally, the present invention encompasses DNA segments that are complementary, or essentially complementary, to target sequences. Nucleic acid sequences that are “complementary” are those that are capable of base-pairing according to the standard Watson-Crick complementary rules. As used herein, the term “complementary sequences” means nucleic acid sequences that are substantially complementary, as may be assessed by the same nucleotide comparison set forth above, or as defined as being capable of hybridizing to a target nucleic acid segment under relatively stringent conditions such as those described herein. These probes may span hundreds or thousands of base pairs.

Alternatively, the hybridizing segments may be shorter oligonucleotides. Sequences of 17 bases long should occur only once in the human genome and, therefore, suffice to specify a unique target sequence. Although shorter oligomers are easier to make and increase in vivo accessibility, numerous other factors are involved in determining the specificity of hybridization. Both binding affinity and sequence specificity of an oligonucleotide to its complementary target increases with increasing length. It is contemplated that exemplary oligonucleotides of about 8, 9, 10, 11, 12, 13, 14, 15, 16, 17, 18, 19, 20, 25, 30, 35, 40, 45, 50, 55, 60, 65, 70, 75, 80, 85, 90, 95, 100, 250, 500, 700, 722, 900, 992, 1000, 1500, 2000, 2500, 2800, 3000, 3500, 3800, 4000, 5000 or more base pairs will be used, although others are contemplated. As mentioned above, longer polynucleotides encoding 10,000, 50,000, 100,000, 150,00, 200,000, 250,000, 300,000 and 500,000 bases are contemplated. Such oligonucleotides and polynucleotides will find use, for example, as probes in FISH, Southern and Northern blots and as primers in amplification reactions.

It will be understood that this invention is not limited to the particular probes disclosed herein and particularly is intended to encompass at least nucleic acid sequences that are hybridizable to the disclosed sequences or are functional sequence analogs of these sequences. For example, a partial sequence may be used to identify a structurally-related gene or the full length genomic or cDNA clone from which it is derived. Those of skill in the art are well aware of the methods for generating cDNA and genomic libraries which can be used as a target for the above-described probes (Sambrook et al., 1989).

For applications in which the nucleic acid segments of the present invention are incorporated into vectors, such as plasmids, cosmids or viruses, these segments may be combined with other DNA sequences, such as promoters, polyadenylation signals, restriction enzyme sites, multiple cloning sites, other coding segments, and the like, such that their overall length may vary considerably. It is contemplated that a nucleic acid fragment of almost any length may be employed, with the total length preferably being limited by the ease of preparation and use in the intended recombinant DNA protocol.

DNA segments encoding a specific gene may be introduced into recombinant host cells and employed for expressing a specific structural or regulatory protein. Alternatively, through the application of genetic engineering techniques, subportions or derivatives of selected genes may be employed. Upstream regions containing regulatory regions such as promoter regions may be isolated and subsequently employed for expression of the selected gene.

B. Labeling of Probes

In certain embodiments, it will be advantageous to employ nucleic acid sequences of the present invention in combination with an appropriate means, such as a label, for determining hybridization. A wide variety of appropriate indicator means are known in the art, including fluorescent, radioactive, chemiluminescent, electroluminescent, enzymatic tag or other ligands, such as avidin/biotin, antibodies, affinity labels, etc., which are capable of being detected. In preferred embodiments, one may desire to employ a fluorescent label such as digoxigenin, spectrum orange, fluorosein, eosin, an acridine dye, a rhodamine, Alexa 350, Alexa 430, AMCA, BODIPY 630/650, BODIPY 650/665, BODIPY-FL, BODIPY-R6G, BODIPY-TMR, BODIPY-TRX, cascade blue, Cy2, Cy3, Cy5,6-FAM, HEX, 6-JOE, Oregon green 488, Oregon green 500, Oregon green 514, pacific blue, REG, ROX, TAMRA, TET, or Texas red.

In the case of enzyme tags such as urease alkaline phosphatase or peroxidase, colorimetric indicator substrates are known which can be employed to provide a detection means visible to the human eye or spectrophotometrically, to identify specific hybridization with complementary nucleic acid-containing samples. Examples of affinity labels include but are not limited to the following: an antibody, an antibody fragment, a receptor protein, a hormone, biotin, DNP, or any polypeptide/protein molecule that binds to an affinity label and may be used for separation of the amplified gene.

The indicator means may be attached directly to the probe, or it may be attached through antigen bonding. In preferred embodiments, digoxigenin is attached to the probe before denaturization and a fluorophore labeled anti-digoxigenin FAB fragment is added after hybridization.

C. Hybridization Conditions

Suitable hybridization conditions will be well known to those of skill in the art. Conditions may be rendered less stringent by increasing salt concentration and decreasing temperature. For example, a medium stringency condition could be provided by about 0.1 to 0.25 M NaCl at temperatures of about 37° C. to about 55° C., while a low stringency condition could be provided by about 0.15 M to about 0.9 M salt, at temperatures ranging from about 20° C. to about 55° C. Thus, hybridization conditions can be readily manipulated, and thus will generally be a method of choice depending on the desired results.

In other embodiments, hybridization may be achieved under conditions of, for example, 50 mM Tris-HCl (pH 8.3), 75 mM KCl, 3 mM MgCl₂, 10 mM dithiothreitol, at temperatures between approximately 20° C. to about 37° C. Other hybridization conditions utilized could include approximately 10 mM Tris-HCl (pH 8.3), 50 mM KCl, 1.5 μM MgCl₂, at temperatures ranging from approximately 40° C. to about 72° C. Formamide and SDS also may be used to alter the hybridization conditions.

### VI. BIOMARKERS AND OTHER RISK FACTORS

Various biomarkers of prognostic significance can be used in conjunction with the specific nucleic acid probes discussed above. These biomarkers could aid in predicting the survival in low stage cancers and the progression from preneoplastic lesions to invasive lung cancer. These markers can include proliferation activity as measured by Ki-67 (MIB1), angiogenesis as quantitated by expression of VEGF and microvessels using CD34, oncogene expression as measured by erb B2, and loss of tumor suppresser genes as measured by p53 expression.

Multiple biomarker candidates have been implicated in the evolution of neoplastic lung lesions. Bio-markers that have been studies include general genomic markers including chromosomal alterations, specific genomic markers such as alterations in proto-oncogenes such as K-Ras, Erb, 1/EGFR, Cyclin D; proliferation markers such as Ki67 or PCNA, squamous differentiation markers, and nuclear retinoid receptors (Papadimitrakopoulou et al., 1996) The latter are particularly interesting as they may be modulated by specific chemopreventive drugs such as 13-cis-retinoic acid or 4HPR and culminate in apoptosis of the defective cells with restoration of a normally differentiated mucosa (Zou et al., 1998).

A. Tumor Angiogenesis by Microvessel Counts

Tumor angiogenesis can be quantitated by microvessel density and is a viable prognostic factor in stage 1 NSCLC. Tumor microvessel density appears to be a good predictor of survival in stage 1 NSCLC.

B. Vascular Endothelial Growth Factor (VEGF)

VEGF (3, 6-8 ch 4) an endothelial cell specific mitogen is an important regulator of tumor angiogenesis who's expression correlates well with lymph node metastases and is a good indirect indicator of tumor agniogenesis. VEGF in turn is upregulated by P53 protein accumulation in NSCLC.

C. p53

The role of p53 mutations in predicting progression and survival of patients with NSCLC is widely debated. Although few studies imply a negligible role, the majority of the studies provide compelling evidence regarding the role of p53 as one of the prognostic factors in NSCLC. The important role of p53 in the biology of NSCLC has been the basis for adenovirus mediated p53 gene transfer in patients with advanced NSCLC (Carcy et al., 1980). In addition p53 has also been shown to be an independent predictor of chemotherapy response in NSCLC. In a recent study (Vallmer et al., 1985), the importance of p53 accumulation in preinvasive bronchial lesions from patients with lung cancer and those who did not progress to cancer were studied. It was demonstrated that p53 accumulation in preneoplastic lesions had a higher rate of progression to invasion than did p53 negative lesions.

D. c-erb-B2

Similar to p53, c-erg-B2 (Her2/neu) expression has also been shown to be a good marker of metastatic propensity and an indicator of survival in these tumors.

E. Ki-67 Proliferation Marker

In addition to the above markers, tumor proliferation index as measured by the extent of labeling of tumor cells for Ki-67, a nuclear antigen expressed throughout cell cycle correlates significantly with clinical outcome in Stage 1 NSCLC (Feinstein et al., 1970). The higher the tumor proliferation index the poorer is the disease free survival labeling indices provides significant complementary, if not independent prognostic information in Stage 1 NSCLC, and helps in the identification of a subset of patients with Stage 1 NSCLC who may need more aggressive therapy.

Alterations in the 3p21.3 and 10q22 loci are known to be associated with a number of cancers. More specifically, point mutations, deletions, insertions or regulatory perturbations relating to the 3p21.3 and 10q22 loci may cause cancer or promote cancer development, cause or promoter tumor progression at a primary site, and/or cause or promote metastasis. Other phenomena at the 3p21.3 and 10q22 loci include angiogenesis and tissue invasion. Thus, the present inventors have demonstrated that deletions at 3p21.3 and 10q22 can be used not only as a diagnostic or prognostic indicator of cancer, but to predict specific events in cancer development, progression and therapy.

A variety of different assays are contemplated in this regard, including but not limited to, fluorescent in situ hybridization (FISH), direct DNA sequencing, PFGE analysis, Southern or Northern blotting, single-stranded conformation analysis (SSCA), RNase protection assay, allele-specific oligonucleotide (ASO), dot blot analysis, denaturing gradient gel electrophoresis, RFLP and PCR-SSCP.

Various types of defects are to be identified. Thus, “alterations” should be read as including deletions, insertions, point mutations and duplications. Point mutations result in stop codons, frameshift mutations or amino acid substitutions. Somatic mutations are those occurring in non-germline tissues. Germ-line tissue can occur in any tissue and are inherited.

F. Surfactant Protein A

There are four main surfactant proteins: SP-A, B, C, and D. SP-A and D are hydrophilic, while SP-B and C are hydrophobic. The proteins are very sensitive to experimental conditions (temperature, pH, concentration, substances such as calcium, and so on). Moreover, their effects tend to overlap and thus it is difficult to pinpoint the specific role of each protein.

SP-A was the first surfactant protein to be identified, and is also the most abundant (Ingenito et al., 1999). Its molecular mass varies from 26-38 kDa. (Pérez-Gil et al., 1998). The protein has a “bouquet” structure of six trimers (Haagsman and Diemel, 2001), and can be found in an open or closed form depending on the other substances present in the system. Calcium ions produce the closed-bouquet form. (Palaniyar et al., 1998).

SP-A plays a role in immune defense. It is also involved in surfactant transport/adsorption (with other proteins). SP-A is necessary for the production of tubular myelin, a lipid transport structure unique to the lungs. Tubular myelin consists of square tubes of lipid lined with protein (Palaniyar et al., 2001). Mice genetically engineered to lack SP-A have normal lung structure and surfactant function, and it is possible that SP-A's beneficial surfactant properties are only evident under situations of stress (Korfhagen et al., 1996).

G. Patient Interview and Other Risk Factors

In addition to analyzing the presence or absence of polymorphisms, as discussed above, it my be desirable to evaluate additional factors in a patient. For example, a patient interview, which would include a smoking history (years smoking, pack/day, etc.) is highly relevant to the diagnosis/prognosis. Also, the presence or absence of morphologic changes in sputum cells (squamous metaplasia, dysplasia, etc.) and a genetic instability score (genetic instability=composing the sum of abnormalities from various combinations in epithelial and neutrophils in sputum and/or peripheral blood cells or bone marrow cells or stem cells isolated from blood or bone marrow) may be used.

### VII. SAMPLES

In accordance with the present invention, one will obtain a biological sample that contains nucleic acids. Various embodiments include paraffin imbedded tissue, frozen tissue, surgical fine needle aspirations, cells of the skin, muscle, lung, head and neck, esophagus, kidney, pancreas, mouth, throat, pharynx, larynx, esophagus, facia, brain, prostate, breast, endometrium, small intestine, blood cells, liver, testes, ovaries, colon, skin, stomach, spleen, lymph node, bone marrow or kidney. Other embodiments include fluid samples such as bronchial brushes, bronchial washes, bronchial lavages, peripheral blood, lymph fluid, ascites, serous fluid, pleural effusion, sputum, cerebrospinal fluid, lacrimal fluid, esophageal washes, stool or urinary specimens such as bladder washing and urine.

Bronchial washes sample more area of bronchial epithelium but are also frequently cytologically normal. A more complete sampling of the respiratory passages may occur with a bronchiolar alveolar lavage in which both left and right proximal and distal small bronchi and bronchioles are washed out.

Nucleic acids are isolated from cells contained in the biological sample, according to standard methodologies (Sambrook et al., 1989). The nucleic acid may be genomic DNA or fractionated or whole cell RNA. Where RNA is used, it may be desired to convert the RNA to a complementary DNA. Depending on the format, the specific nucleic acid of interest is identified in the sample directly using amplification or with a second, known nucleic acid following amplification.

Following detection, one may compare the results seen in a given sample with a statistically significant reference group of samples from normal patients and patients that have or lack alterations in the various chromosome loci and control regions. In this way, one then correlates the amount or kind of alterations detected with various clinical states and treatment options.

### VIII. THERAPIES

The present invention envisions the use of assays to detect cancer and predict its progression in conjunction with cancer therapies. In some cases, where patients are suspected to be at risk of cancer, prophylactic treatments may be employed. In other cancer subjects, diagnosis may permit permit early therapeutic intervention. In yet other situations, the result of the assays described herein may provide useful information regarding the need for repeated treatments, for example, where there is a likelihood of metastatic, recurrent or residual disease. Finally, the present invention may prove useful in demonstrating which therapies do and do not provide benefit to a particular patient. The following is a non-limiting list of such treatments.

A. Chemotherapy

A wide variety of chemotherapeutic agents may be used in accordance with the present invention. The term “chemotherapy” refers to the use of drugs to treat cancer. A “chemotherapeutic agent” is used to connote a compound or composition that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis. Most chemotherapeutic agents fall into the following categories: alkylating agents, antimetabolites, antitumor antibiotics, mitotic inhibitors, and nitrosoureas.

1. Alkylating Agents

Alkylating agents are drugs that directly interact with genomic DNA to prevent the cancer cell from proliferating. This category of chemotherapeutic drugs represents agents that affect all phases of the cell cycle, that is, they are not phase-specific. Alkylating agents can be implemented to treat chronic leukemia, non-Hodgkin's lymphoma, Hodgkin's disease, multiple myeloma, and particular cancers of the breast, lung, and ovary. They include: busulfan, chlorambucil, cisplatin, cyclophosphamide (cytoxan), dacarbazine, ifosfamide, mechlorethamine (mustargen), and melphalan. Troglitazaone can be used to treat cancer in combination with any one or more of these alkylating agents, some of which are discussed below.

a. Busulfan

Busulfan (also known as myleran) is a bifunctional alkylating agent. Busulfan is known chemically as 1,4-butanediol dimethanesulfonate.

Busulfan is not a structural analog of the nitrogen mustards. Busulfan is available in tablet form for oral administration. Each scored tablet contains 2 mg busulfan and the inactive ingredients magnesium stearate and sodium chloride.

Busulfan is indicated for the palliative treatment of chronic myelogenous (myeloid, myelocytic, granulocytic) leukemia. Although not curative, busulfan reduces the total granulocyte mass, relieves symptoms of the disease, and improves the clinical state of the patient. Approximately 90% of adults with previously untreated chronic myelogenous leukemia will obtain hematologic remission with regression or stabilization of organomegaly following the use of busulfan. It has been shown to be superior to splenic irradiation with respect to survival times and maintenance of hemoglobin levels, and to be equivalent to irradiation at controlling splenomegaly.

b. Chlorambucil

Chlorambucil (also known as leukeran) is a bifunctional alkylating agent of the nitrogen mustard type that has been found active against selected human neoplastic diseases. Chlorambucil is known chemically as 4-[bis(2-chlorethyl)amino]benzenebutanoic acid.

Chlorambucil is available in tablet form for oral administration. It is rapidly and completely absorbed from the gastrointestinal tract. After single oral doses of 0.6-1.2 mg/kg, peak plasma chlorambucil levels are reached within one hour and the terminal half-life of the parent drug is estimated at 1.5 hours. 0.1 to 0.2 mg/kg/day or 3 to 6 mg/m²/day or alternatively 0.4 mg/kg may be used for antineoplastic treatment. Treatment regimes are well know to those of skill in the art and can be found in the “Physicians Desk Reference” and in “Remington's Pharmaceutical Sciences” referenced herein.

Chlorambucil is indicated in the treatment of chronic lymphatic (lymphocytic) leukemia, malignant lymphomas including lymphosarcoma, giant follicular lymphoma and Hodgkin's disease. It is not curative in any of these disorders but may produce clinically useful palliation. Thus, it can be used in combination with troglitazone in the treatment of cancer.

C. Cisplatin

Cisplatin has been widely used to treat cancers such as metastatic testicular or ovarian carcinoma, advanced bladder cancer, head or neck cancer, cervical cancer, lung cancer or other tumors. Cisplatin can be used alone or in combination with other agents, with efficacious doses used in clinical applications of 15-20 mg/m² for 5 days every three weeks for a total of three courses. Exemplary doses may be 0.50 mg/m², 10 mg/m², 1.50 mg/m², 1.75 mg/m², 2.0 mg/m², 3.0 mg/m², 4.0 mg/m², 5.0 mg/m², 10 mg//m². Of course, all of these dosages are exemplary, and any dosage in-between these points is also expected to be of use in the invention.

Cisplatin is not absorbed orally and must therefore be delivered via injection intravenously, subcutaneously, intratumorally or intraperitoneally.

d. Cyclophosphamide

Cyclophosphamide is 2H-1,3,2-Oxazaphosphorin-2-amine, N,N-bis(2-chloroethyl)tetrahydro-, 2-oxide, monohydrate; termed Cytoxan available from Mead Johnson; and Neosar available from Adria. Cyclophosphamide is prepared by condensing 3-amino-1-propanol with N,N-bis(2-chlorethyl)phosphoramidic dichloride[(ClCH₂CH₂)₂N—POCl₂] in dioxane solution under the catalytic influence of triethylamine. The condensation is double, involving both the hydroxyl and the amino groups, thus effecting the cyclization.

Unlike other β-chloroethylamino alkylators, it does not cyclize readily to the active ethyleneimonium form until activated by hepatic enzymes. Thus, the substance is stable in the gastrointestinal tract, tolerated well and effective by the oral and parental routes and does not cause local vesication, necrosis, phlebitis or even pain.

Suitable doses for adults include, orally, 1 to 5 mg/kg/day (usually in combination), depending upon gastrointestinal tolerance; or 1 to 2 mg/kg/day; intravenously, initially 40 to 50 mg/kg in divided doses over a period of 2 to 5 days or 10 to 15 mg/kg every 7 to 10 days or 3 to 5 mg/kg twice a week or 1.5 to 3 mg/kg/day. A dose 250 mg/kg/day may be administered as an antineoplastic. Because of gastrointestinal adverse effects, the intravenous route is preferred for loading. During maintenance, a leukocyte count of 3000 to 4000/mm³ usually is desired. The drug also sometimes is administered intramuscularly, by infiltration or into body cavities. It is available in dosage forms for injection of 100, 200 and 500 mg, and tablets of 25 and 50 mg the skilled artisan is referred to “Remington's Pharmaceutical Sciences” 15th Edition, chapter 61, incorporate herein as a reference, for details on doses for administration.

e. Melphalan

Melphalan, also known as alkeran, L-phenylalanine mustard, phenylalanine mustard, L-PAM, or L-sarcolysin, is a phenylalanine derivative of nitrogen mustard. Melphalan is a bifunctional alkylating agent which is active against selective human neoplastic diseases. It is known chemically as 4-[bis(2-chloroethyl)amino]-L-phenylalanine.

Melphalan is the active L-isomer of the compound and was first synthesized in 1953 by Bergel and Stock; the D-isomer, known as medphalan, is less active against certain animal tumors, and the dose needed to produce effects on chromosomes is larger than that required with the L-isomer. The racemic (DL-) form is known as merphalan or sarcolysin. Melphalan is insoluble in water and has a pKa₁ of ˜2.1. Melphalan is available in tablet form for oral administration and has been used to treat multiple myeloma.

Available evidence suggests that about one third to one half of the patients with multiple myeloma show a favorable response to oral administration of the drug.

Melphalan has been used in the treatment of epithelial ovarian carcinoma. One commonly employed regimen for the treatment of ovarian carcinoma has been to administer melphalan at a dose of 0.2 mg/kg daily for five days as a single course. Courses are repeated every four to five weeks depending upon hematologic tolerance (Smith and Rutledge, 1975; Young et al., 1978). Alternatively the dose of melphalan used could be as low as 0.05 mg/kg/day or as high as 3 mg/kg/day or any dose in between these doses or above these doses. Some variation in dosage will necessarily occur depending on the condition of the subject being treated. The person responsible for administration will, in any event, determine the appropriate dose for the individual subject.

2. Antimetabolites

Antimetabolites disrupt DNA and RNA synthesis. Unlike alkylating agents, they specifically influence the cell cycle during S phase. They have used to combat chronic leukemias in addition to tumors of breast, ovary and the gastrointestinal tract. Antimetabolites include 5-fluorouracil (5-FU), cytarabine (Ara-C), fludarabine, gemcitabine, and methotrexate.

5-Fluorouracil (5-FU) has the chemical name of 5-fluoro-2,4(1H,3H)-pyrimidinedione. Its mechanism of action is thought to be by blocking the methylation reaction of deoxyuridylic acid to thymidylic acid. Thus, 5-FU interferes with the synthesis of deoxyribonucleic acid (DNA) and to a lesser extent inhibits the formation of ribonucleic acid (RNA). Since DNA and RNA are essential for cell division and proliferation, it is thought that the effect of 5-FU is to create a thymidine deficiency leading to cell death. Thus, the effect of 5-FU is found in cells that rapidly divide, a characteristic of metastatic cancers.

3. Antitumor Antibiotics

Antitumor antibiotics have both antimicrobial and cytotoxic activity. These drugs also interfere with DNA by chemically inhibiting enzymes and mitosis or altering cellular membranes. These agents are not phase specific so they work in all phases of the cell cycle. Thus, they are widely used for a variety of cancers. Examples of antitumor antibiotics include bleomycin, dactinomycin, daunorubicin, doxorubicin (Adriamycin), and idarubicin, some of which are discussed in more detail below. Widely used in clinical setting for the treatment of neoplasms these compounds are administered through bolus injections intravenously at doses ranging from 25-75 mg/m² at 21 day intervals for adriamycin, to 35-100 mg/m² for etoposide intravenously or orally.

a. Doxorubicin

Doxorubicin hydrochloride, 5,12-Naphthacenedione, (8s-cis)-10-[(3-amino-2,3,6-trideoxy-a-L-lyxo-hexopyranosyl)oxy]-7,8,9,10-tetrahydro-6,8,11-trihydroxy-8-(hydroxyacetyl)-1-methoxy-hydrochloride (hydroxydaunorubicin hydrochloride, Adriamycin) is used in a wide antineoplastic spectrum. It binds to DNA and inhibits nucleic acid synthesis, inhibits mitosis and promotes chromosomal aberrations.

Administered alone, it is the drug of first choice for the treatment of thyroid adenoma and primary hepatocellular carcinoma. It is a component of 31 first-choice combinations for the treatment of ovarian, endometrial and breast tumors, bronchogenic oat-cell carcinoma, non-small cell lung carcinoma, gastric adenocarcinoma, retinoblastoma, neuroblastoma, mycosis fungoides, pancreatic carcinoma, prostatic carcinoma, bladder carcinoma, myeloma, diffuse histiocytic lymphoma, Wilms' tumor, Hodgkin's disease, adrenal tumors, osteogenic sarcoma soft tissue sarcoma, Ewing's sarcoma, rhabdomyosarcoma and acute lymphocytic leukemia. It is an alternative drug for the treatment of islet cell, cervical, testicular and adrenocortical cancers. It is also an immunosuppressant.

Doxorubicin is absorbed poorly and must be administered intravenously. The pharmacokinetics are multicompartmental. Distribution phases have half-lives of 12 minutes and 3.3 hr. The elimination half-life is about 30 hr. Forty to 50% is secreted into the bile. Most of the remainder is metabolized in the liver, partly to an active metabolite (doxorubicinol), but a few percent is excreted into the urine. In the presence of liver impairment, the dose should be reduced.

Appropriate doses are, intravenous, adult, 60 to 75 mg/m² at 21-day intervals or 25 to 30 mg/m² on each of 2 or 3 successive days repeated at 3- or 4-wk intervals or 20 mg/m² once a week. The lowest dose should be used in elderly patients, when there is prior bone-marrow depression caused by prior chemotherapy or neoplastic marrow invasion, or when the drug is combined with other myelopoietic suppressant drugs. The dose should be reduced by 50% if the serum bilirubin lies between 1.2 and 3 mg/dL and by 75% if above 3 mg/dL. The lifetime total dose should not exceed 550 mg/m² in patients with normal heart function and 400 mg/m² in persons having received mediastinal irradiation. Alternatively, 30 mg/m² on each of 3 consecutive days, repeated every 4 wk. Exemplary doses may be 10 mg/m², 20 mg/m², 30 mg/m², 50 mg/m², 100 mg/m², 150 mg/m², 175 mg/m², 200 mg/m², 225 mg/m², 250 mg/m², 275 mg/m², 300 mg/m², 350 mg/m², 400 mg/m², 425 mg/m², 450 mg/m², 475 mg/m², 500 mg/m². Of course, all of these dosages are exemplary, and any dosage in-between these points is also expected to be of use in the invention.

In the present invention the inventors have employed troglitazone as an exemplary chemotherapeutic agent to synergistically enhance the antineoplastic effects of the doxorubicin in the treatment of cancers. Those of skill in the art will be able to use the invention as exemplified potentiate the effects of doxorubicin in a range of different pre-cancer and cancers.

b. Daunorubicin

Daunorubicin hydrochloride, 5,12-Naphthacenedione, (8S-cis)-8-acetyl-10-[(3-amino-2,3,6-trideoxy-a-L-lyxo-hexanopyranosyl)oxy]-7,8,9,10-tetrahydro-6,8,11-trihydroxy-10-methoxy-, hydrochloride; also termed cerubidine and available from Wyeth. Daunorubicin intercalates into DNA, blocks DAN-directed RNA polymerase and inhibits DNA synthesis. It can prevent cell division in doses that do not interfere with nucleic acid synthesis.

In combination with other drugs it is included in the first-choice chemotherapy of acute myelocytic leukemia in adults (for induction of remission), acute lymphocytic leukemia and the acute phase of chronic myelocytic leukemia. Oral absorption is poor, and it must be given intravenously. The half-life of distribution is 45 minutes and of elimination, about 19 hr. The half-life of its active metabolite, daunorubicinol, is about 27 hr. Daunorubicin is metabolized mostly in the liver and also secreted into the bile (ca 40%). Dosage must be reduced in liver or renal insufficiencies.

Suitable doses are (base equivalent), intravenous adult, younger than 60 yr. 45 mg/m²/day (30 mg/m² for patients older than 60 yr.) for 1, 2 or 3 days every 3 or 4 wk or 0.8 mg/kg/day for 3 to 6 days every 3 or 4 wk; no more than 550 mg/m² should be given in a lifetime, except only 450 mg/m² if there has been chest irradiation; children, 25 mg/m² once a week unless the age is less than 2 yr. or the body surface less than 0.5 m, in which case the weight-based adult schedule is used. It is available in injectable dosage forms (base equivalent) 20 mg (as the base equivalent to 21.4 mg of the hydrochloride). Exemplary doses may be 10 mg/m², 20 mg/m², 30 mg/m², 50 mg/m², 100 mg/m², 150 mg/m², 175 mg/m², 200 mg/m², 225 mg/m², 250 mg/m², 275 mg/m², 300 mg/m², 350 mg/m², 400 mg/m², 425 mg/m², 450 mg/m², 475 mg/m², 500 mg/m². Of course, all of these dosages are exemplary, and any dosage in-between these points is also expected to be of use in the invention.

C. Mitomycin

Mitomycin (also known as mutamycin and/or mitomycin-C) is an antibiotic isolated from the broth of Streptomyces caespitosus which has been shown to have antitumor activity. The compound is heat stable, has a high melting point, and is freely soluble in organic solvents.

Mitomycin selectively inhibits the synthesis of deoxyribonucleic acid (DNA). The guanine and cytosine content correlates with the degree of mitomycin-induced cross-linking. At high concentrations of the drug, cellular RNA and protein synthesis are also suppressed.

In humans, mitomycin is rapidly cleared from the serum after intravenous administration. Time required to reduce the serum concentration by 50% after a 30 mg. bolus injection is 17 minutes. After injection of 30 mg., 20 mg., or 10 mg. I.V., the maximal serum concentrations were 2.4 mg./mL, 1.7 mg./mL, and 0.52 mg./mL, respectively. Clearance is effected primarily by metabolism in the liver, but metabolism occurs in other tissues as well. The rate of clearance is inversely proportional to the maximal serum concentration because, it is thought, of saturation of the degradative pathways. Approximately 10% of a dose of mitomycin is excreted unchanged in the urine. Since metabolic pathways are saturated at relatively low doses, the percent of a dose excreted in urine increases with increasing dose. In children, excretion of intravenously administered mitomycin is similar.

d. Actinomycin D

Actinomycin D (Dactinomycin) [50-76-0]; C₆₂H₈₆N₁₂O₁₆ (1255.43) is an antineoplastic drug that inhibits DNA-dependent RNA polymerase. It is a component of first-choice combinations for treatment of choriocarcinoma, embryonal rhabdomyosarcoma, testicular tumor and Wilms' tumor. Tumors that fail to respond to systemic treatment sometimes respond to local perfusion. Dactinomycin potentiates radiotherapy. It is a secondary (efferent) immunosuppressive.

Actinomycin D is used in combination with primary surgery, radiotherapy, and other drugs, particularly vincristine and cyclophosphamide. Antineoplastic activity has also been noted in Ewing's tumor, Kaposi's sarcoma, and soft-tissue sarcomas. Dactinomycin can be effective in women with advanced cases of choriocarcinoma. It also produces consistent responses in combination with chlorambucil and methotrexate in patients with metastatic testicular carcinomas. A response may sometimes be observed in patients with Hodgkin's disease and non-Hodgkin's lymphomas. Dactinomycin has also been used to inhibit immunological responses, particularly the rejection of renal transplants.

Half of the dose is excreted intact into the bile and 10% into the urine; the half-life is about 36 hr. The drug does not pass the blood-brain barrier. Actinomycin D is supplied as a lyophilized powder (0/5 mg in each vial). The usual daily dose is 10 to 15 mg/kg; this is given intravenously for 5 days; if no manifestations of toxicity are encountered, additional courses may be given at intervals of 3 to 4 weeks. Daily injections of 100 to 400 mg have been given to children for 10 to 14 days; in other regimens, 3 to 6 mg/kg, for a total of 125 mg/kg, and weekly maintenance doses of 7.5 mg/kg have been used. Although it is safer to administer the drug into the tubing of an intravenous infusion, direct intravenous injections have been given, with the precaution of discarding the needle used to withdraw the drug from the vial in order to avoid subcutaneous reaction. Exemplary doses may be 100 mg/m², 150 mg/m², 175 mg/m², 200 mg/m², 225 mg/m², 250 mg/m², 275 mg/m², 300 mg/m², 350 mg/m², 400 mg/m², 425 mg/m², 450 mg/m², 475 mg/m², 500 mg/m². Of course, all of these dosages are exemplary, and any dosage in-between these points is also expected to be of use in the invention.

e. Bleomycin

Bleomycin is a mixture of cytotoxic glycopeptide antibiotics isolated from a strain of Streptomyces verticillus. Although the exact mechanism of action of bleomycin is unknown, available evidence would seem to indicate that the main mode of action is the inhibition of DNA synthesis with some evidence of lesser inhibition of RNA and protein synthesis.

In mice, high concentrations of bleomycin are found in the skin, lungs, kidneys, peritoneum, and lymphatics. Tumor cells of the skin and lungs have been found to have high concentrations of bleomycin in contrast to the low concentrations found in hematopoietic tissue. The low concentrations of bleomycin found in bone marrow may be related to high levels of bleomycin degradative enzymes found in that tissue.

In patients with a creatinine clearance of >35 mL per minute, the serum or plasma terminal elimination half-life of bleomycin is approximately 115 minutes. In patients with a creatinine clearance of <35 mL per minute, the plasma or serum terminal elimination half-life increases exponentially as the creatinine clearance decreases. In humans, 60% to 70% of an administered dose is recovered in the urine as active bleomycin. Bleomycin may be given by the intramuscular, intravenous, or subcutaneous routes. It is freely soluble in water.

Bleomycin should be considered a palliative treatment. It has been shown to be useful in the management of the following neoplasms either as a single agent or in proven combinations with other approved chemotherapeutic agents in squamous cell carcinoma such as head and neck (including mouth, tongue, tonsil, nasopharynx, oropharynx, sinus, palate, lip, buccal mucosa, gingiva, epiglottis, larynx), skin, penis, cervix, and vulva. It has also been used in the treatment of lymphomas and testicular carcinoma.

Because of the possibility of an anaphylactoid reaction, lymphoma patients should be treated with two units or less for the first two doses. If no acute reaction occurs, then the regular dosage schedule may be followed.

Improvement of Hodgkin's Disease and testicular tumors is prompt and noted within 2 weeks. If no improvement is seen by this time, improvement is unlikely. Squamous cell cancers respond more slowly, sometimes requiring as long as 3 weeks before any improvement is noted.

4. Mitotic Inhibitors

Mitotic inhibitors include plant alkaloids and other natural agents that can inhibit either protein synthesis required for cell division or mitosis. They operate during a specific phase during the cell cycle. Mitotic inhibitors comprise docetaxel, etoposide (VP16), paclitaxel, taxol, taxotere, vinblastine, vincristine, and vinorelbine.

a. Etoposide (VP16)

VP16 is also known as etoposide and is used primarily for treatment of testicular tumors, in combination with bleomycin and cisplatin, and in combination with cisplatin for small-cell carcinoma of the lung. It is also active against non-Hodgkin's lymphomas, acute nonlymphocytic leukemia, carcinoma of the breast, and Kaposi's sarcoma associated with acquired immunodeficiency syndrome (AIDS).

VP16 is available as a solution (20 mg/ml) for intravenous administration and as 50-mg, liquid-filled capsules for oral use. For small-cell carcinoma of the lung, the intravenous dose (in combination therapy) is can be as much as 100 mg/m² or as little as 2 mg/m², routinely 35 mg/m², daily for 4 days, to 50 mg/m², daily for 5 days have also been used. When given orally, the dose should be doubled. Hence the doses for small cell lung carcinoma may be as high as 200-250 mg/m². The intravenous dose for testicular cancer (in combination therapy) is 50 to 100 mg/m² daily for 5 days, or 100 mg/m² on alternate days, for three doses. Cycles of therapy are usually repeated every 3 to 4 weeks. The drug should be administered slowly during a 30- to 60-minute infusion in order to avoid hypotension and bronchospasm, which are probably due to the solvents used in the formulation.

b. Taxol

Taxol is an experimental antimitotic agent, isolated from the bark of the ash tree, Taxus brevifolia. It binds to tubulin (at a site distinct from that used by the vinca alkaloids) and promotes the assembly of microtubules. Taxol is currently being evaluated clinically; it has activity against malignant melanoma and carcinoma of the ovary. Maximal doses are 30 mg/m² per day for 5 days or 210 to 250 mg/m² given once every 3 weeks. Of course, all of these dosages are exemplary, and any dosage in-between these points is also expected to be of use in the invention.

C. Vinblastine

Vinblastine is another example of a plant alkyloid that can be used in combination with troglitazone for the treatment of cancer and precancer. When cells are incubated with vinblastine, dissolution of the microtubules occurs.

Unpredictable absorption has been reported after oral administration of vinblastine or vincristine. At the usual clinical doses the peak concentration of each drug in plasma is approximately 0.4 mM. Vinblastine and vincristine bind to plasma proteins. They are extensively concentrated in platelets and to a lesser extent in leukocytes and erythrocytes.

After intravenous injection, vinblastine has a multiphasic pattern of clearance from the plasma; after distribution, drug disappears from plasma with half-lives of approximately 1 and 20 hours. Vinblastine is metabolized in the liver to biologically activate derivative desacetylvinblastine. Approximately 15% of an administered dose is detected intact in the urine, and about 10% is recovered in the feces after biliary excretion. Doses should be reduced in patients with hepatic dysfunction. At least a 50% reduction in dosage is indicated if the concentration of bilirubin in plasma is greater than 3 mg/dl (about 50 mM).

Vinblastine sulfate is available in preparations for injection. The drug is given intravenously; special precautions must be taken against subcutaneous extravasation, since this may cause painful irritation and ulceration. The drug should not be injected into an extremity with impaired circulation. After a single dose of 0.3 mg/kg of body weight, myelosuppression reaches its maximum in 7 to 10 days. If a moderate level of leukopenia (approximately 3000 cells/mm³) is not attained, the weekly dose may be increased gradually by increments of 0.05 mg/kg of body weight. In regimens designed to cure testicular cancer, vinblastine is used in doses of 0.3 mg/kg every 3 weeks irrespective of blood cell counts or toxicity.

The most important clinical use of vinblastine is with bleomycin and cisplatin in the curative therapy of metastatic testicular tumors. Beneficial responses have been reported in various lymphomas, particularly Hodgkin's disease, where significant improvement may be noted in 50 to 90% of cases. The effectiveness of vinblastine in a high proportion of lymphomas is not diminished when the disease is refractory to alkylating agents. It is also active in Kaposi's sarcoma, neuroblastoma, and Letterer-Siwe disease (histiocytosis X), as well as in carcinoma of the breast and choriocarcinoma in women.

Doses of vinblastine will be determined by the clinician according to the individual patients need. 0.1 to 0.3 mg/kg can be administered or 1.5 to 2 mg/m² can also be administered. Alternatively, 0.1 mg/m², 0.12 mg/m², 0.14 mg/m², 0.15 mg/m², 0.2 mg/m², 0.25 mg/m², 0.5 mg/m², 1.0 mg/m², 1.2 mg/m², 1.4 mg/m², 1.5 mg/m², 2.0 mg/m², 2.5 mg/m², 5.0 mg/m², 6 mg/m², 8 mg/m², 9 mg/m², 10 mg/m², 20 mg/m², can be given. Of course, all of these dosages are exemplary, and any dosage in-between these points is also expected to be of use in the invention.

d. Vincristine

Vincristine blocks mitosis and produces metaphase arrest. It seems likely that most of the biological activities of this drug can be explained by its ability to bind specifically to tubulin and to block the ability of protein to polymerize into microtubules. Through disruption of the microtubules of the mitotic apparatus, cell division is arrested in metaphase. The inability to segregate chromosomes correctly during mitosis presumably leads to cell death.

The relatively low toxicity of vincristine for normal marrow cells and epithelial cells make this agent unusual among anti-neoplastic drugs, and it is often included in combination with other myelosuppressive agents.

Unpredictable absorption has been reported after oral administration of vinblastine or vincristine. At the usual clinical doses the peak concentration of each drug in plasma is approximately 0.4 mM.

Vinblastine and vincristine bind to plasma proteins. They are extensively concentrated in platelets and to a lesser extent in leukocytes and erythrocytes.

Vincristine has a multiphasic pattern of clearance from the plasma; the terminal half-life is about 24 hours. The drug is metabolized in the liver, but no biologically active derivatives have been identified. Doses should be reduced in patients with hepatic dysfunction. At least a 50% reduction in dosage is indicated if the concentration of bilirubin in plasma is greater than 3 mg/dl (about 50 mM).

Vincristine sulfate is available as a solution (1 mg/ml) for intravenous injection. Vincristine used together with corticosteroids is presently the treatment of choice to induce remissions in childhood leukemia; the optimal dosages for these drugs appear to be vincristine, intravenously, 2 mg/m² of body-surface area, weekly, and prednisone, orally, 40 mg/m², daily. Adult patients with Hodgkin's disease or non-Hodgkin's lymphomas usually receive vincristine as a part of a complex protocol. When used in the MOPP regimen, the recommended dose of vincristine is 1.4 mg/m². High doses of vincristine seem to be tolerated better by children with leukemia than by adults, who may experience sever neurological toxicity. Administration of the drug more frequently than every 7 days or at higher doses seems to increase the toxic manifestations without proportional improvement in the response rate. Precautions should also be used to avoid extravasation during intravenous administration of vincristine. Vincristine (and vinblastine) can be infused into the arterial blood supply of tumors in doses several times larger than those that can be administered intravenously with comparable toxicity.

Vincristine has been effective in Hodgkin's disease and other lymphomas. Although it appears to be somewhat less beneficial than vinblastine when used alone in Hodgkin's disease, when used with mechlorethamine, prednisone, and procarbazine (the so-called MOPP regimen), it is the preferred treatment for the advanced stages (III and IV) of this disease. In non-Hodgkin's lymphomas, vincristine is an important agent, particularly when used with cyclophosphamide, bleomycin, doxorubicin, and prednisone. Vincristine is more useful than vinblastine in lymphocytic leukemia. Beneficial response have been reported in patients with a variety of other neoplasms, particularly Wilms' tumor, neuroblastoma, brain tumors, rhabdomyosarcoma, and carcinomas of the breast, bladder, and the male and female reproductive systems.

Doses of vincristine for use will be determined by the clinician according to the individual patients need. 0.01 to 0.03 mg/kg or 0.4 to 1.4 mg/m² can be administered or 1.5 to 2 mg/m² can also be administered. Alternatively 0.02 mg/m², 0.05 mg/m², 0.06 mg/m², 0.07 mg/m², 0.08 mg/m², 0.1 mg/m², 0.12 mg/m², 0.14 mg/m², 0.15 mg/m², 0.2 mg/m², 0.25 mg/m² can be given as a constant intravenous infusion. Of course, all of these dosages are exemplary, and any dosage in-between these points is also expected to be of use in the invention.

e. Camptothecin

Camptothecin is an alkaloid derived from the chinese tree Camptotheca acuminata Decne. Camptothecin and its derivatives are unique in their ability to inhibit DNA Topoisomerase by stabilizing a covalent reaction intermediate, termed “the cleavable complex,” which ultimately causes tumor cell death. It is widely believed that camptothecin analogs exhibited remarkable anti-tumour and anti-leukaemia activity. Application of camptothecin in clinic is limited due to serious side effects and poor water-solubility. At present, some camptothecin analogs (topotecan; irinotecan), either synthetic or semi-synthetic, have been applied to cancer therapy and have shown satisfactory clinical effects. The molecular formula for camptothecin is C₂₀H₁₆N₂O₄, with a molecular weight of 348.36. It is provided as a yellow powder, and may be solubilized to a clear yellow solution at 50 mg/ml in DMSO 1N sodium hydroxide. It is stable for at least two years if stored at 2-8° X in a dry, airtight, light-resistant environment.

5. Nitrosureas

Nitrosureas, like alkylating agents, inhibit DNA repair proteins. They are used to treat non-Hodgkin's lymphomas, multiple myeloma, malignant melanoma, in addition to brain tumors. Examples include carmustine and lomustine.

a. Carmustine

Carmustine (sterile carmustine) is one of the nitrosoureas used in the treatment of certain neoplastic diseases. It is 1,3bis(2-chloroethyl)-1-nitrosourea. It is lyophilized pale yellow flakes or congealed mass with a molecular weight of 214.06. It is highly soluble in alcohol and lipids, and poorly soluble in water. Carmustine is administered by intravenous infusion after reconstitution as recommended. Sterile carmustine is commonly available in 100 mg single dose vials of lyophilized material.

Although it is generally agreed that carmustine alkylates DNA and RNA, it is not cross resistant with other alkylators. As with other nitrosoureas, it may also inhibit several key enzymatic processes by carbamoylation of amino acids in proteins.

Carmustine is indicated as palliative therapy as a single agent or in established combination therapy with other approved chemotherapeutic agents in brain tumors such as glioblastoma, brainstem glioma, medullobladyoma, astrocytoma, ependymoma, and metastatic brain tumors. Also it has been used in combination with prednisone to treat multiple myeloma. Carmustine has proved useful, in the treatment of Hodgkin's Disease and in non-Hodgkin's lymphomas, as secondary therapy in combination with other approved drugs in patients who relapse while being treated with primary therapy, or who fail to respond to primary therapy.

The recommended dose of carmustine as a single agent in previously untreated patients is 150 to 200 mg/m² intravenously every 6 weeks. This may be given as a single dose or divided into daily injections such as 75 to 100 mg/m on 2 successive days. When carmustine is used in combination with other myelosuppressive drugs or in patients in whom bone marrow reserve is depleted, the doses should be adjusted accordingly. Doses subsequent to the initial dose should be adjusted according to the hematologic response of the patient to the preceding dose. It is of course understood that other doses may be used in the present invention for example 10 mg/m², 20 mg/m², 30 mg/m² 40 mg/m² 50 mg/m² 60 mg/m² 70 mg/m² 80 mg/m² 90 mg/m² 100 mg/m². The skilled artisan is directed to, “Remington's Pharmaceutical Sciences” 15th Edition, chapter 61. Some variation in dosage will necessarily occur depending on the condition of the subject being treated. The person responsible for administration will, in any event, determine the appropriate dose for the individual subject.

b. Lomustine

Lomustine is one of the nitrosoureas used in the treatment of certain neoplastic diseases. It is 1-(2-chloro-ethyl)-3-cyclohexyl-1 nitrosourea. It is a yellow powder with the empirical formula of C₉H₁₆ClN₃O₂ and a molecular weight of 233.71. Lomustine is soluble in 10% ethanol (0.05 mg per mL) and in absolute alcohol (70 mg per mL). Lomustine is relatively insoluble in water (<0.05 mg per mL). It is relatively unionized at a physiological pH. Inactive ingredients in lomustine capsules are: magnesium stearate and mannitol.

Although it is generally agreed that lomustine alkylates DNA and RNA, it is not cross resistant with other alkylators. As with other nitrosoureas, it may also inhibit several key enzymatic processes by carbamoylation of amino acids in proteins.

Lomustine may be given orally. Following oral administration of radioactive lomustine at doses ranging from 30 mg/m² to 100 mg/m², about half of the radioactivity given was excreted in the form of degradation products within 24 hours. The serum half-life of the metabolites ranges from 16 hours to 2 days. Tissue levels are comparable to plasma levels at 15 minutes after intravenous administration.

Lomustine has been shown to be useful as a single agent in addition to other treatment modalities, or in established combination therapy with other approved chemotherapeutic agents in both primary and metastatic brain tumors, in patients who have already received appropriate surgical and/or radiotherapeutic procedures. It has also proved effective in secondary therapy against Hodgkin's Disease in combination with other approved drugs in patients who relapse while being treated with primary therapy, or who fail to respond to primary therapy.

The recommended dose of lomustine in adults and children as a single agent in previously untreated patients is 130 mg/m² as a single oral dose every 6 weeks. In individuals with compromised bone marrow function, the dose should be reduced to 100 mg/m² every 6 weeks. When lomustine is used in combination with other myelosuppressive drugs, the doses should be adjusted accordingly. It is understood that other doses may be used for example, 20 mg/m² 30 mg/m², 40 mg/m², 50 mg/m², 60 mg/m², 70 mg/m², 80 mg/m², 90 mg/m², 100 mg/m², 120 mg/m² or any doses between these figures as determined by the clinician to be necessary for the individual being treated.

6. Other Agents

Other agents that may be used include Avastin, Iressa, Erbitux, Velcade, and Gleevec. In addition, growth factor inhibitors and small molecule kinase inhibitors have utility in the present invention as well. All therapies described in Cancer: Principles and Practice of Oncology Single Volume (Book with CD-ROM) by Vincent T. Devita (Editor), Samuel Hellman (Editor), Steven A. Rosenberg (Editor) Lippencott (2001), are hereby incorporated by reference. The following additional therapies are encompassed, as well.

a. Immunotherapy

Immunotherapeutics, generally, rely on the use of immune effector cells and molecules to target and destroy cancer cells. The immune effector may be, for example, an antibody specific for some marker on the surface of a tumor cell. The antibody alone may serve as an effector of therapy or it may recruit other cells to actually effect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells.

Immunotherapy, thus, could be used as part of a combined therapy, in conjunction with Ad-mda7 gene therapy. The general approach for combined therapy is discussed below. Generally, the tumor cell must bear some marker that is amenable to targeting, i.e., is not present on the majority of other cells. Many tumor markers exist and any of these may be suitable for targeting in the context of the present invention. Common tumor markers include carcinoembryonic antigen, prostate specific antigen, urinary tumor associated antigen, fetal antigen, tyrosinase (p97), gp68, TAG-72, HMFG, Sialyl Lewis Antigen, MucA, MucB, PLAP, estrogen receptor, laminin receptor, erb B and p 155.

Tumor Necrosis Factor is a glycoprotein that kills some kinds of cancer cells, activates cytokine production, activates macrophages and endothelial cells, promotes the production of collagen and collagenases, is an inflammatory mediator and also a mediator of septic shock, and promotes catabolism, fever and sleep. Some infectious agents cause tumor regression through the stimulation of TNF production. TNF can be quite toxic when used alone in effective doses, so that the optimal regimens probably will use it in lower doses in combination with other drugs. Its immunosuppressive actions are potentiated by gamma-interferon, so that the combination potentially is dangerous. A hybrid of TNF and interferon-α also has been found to possess anti-cancer activity.

b. Hormonal Therapy

The use of sex hormones according to the methods described herein in the treatment of cancer. While the methods described herein are not limited to the treatment of a specific cancer, this use of hormones has benefits with respect to cancers of the breast, prostate, and endometrial (lining of the uterus). Examples of these hormones are estrogens, anti-estrogens, progesterones, and androgens.

Corticosteroid hormones are useful in treating some types of cancer (lymphoma, leukemias, and multiple myeloma). Corticosteroid hormones can increase the effectiveness of other chemotherapy agents, and consequently, they are frequently used in combination treatments. Prednisone and dexamethasone are examples of corticosteroid hormones.

B. Radiotherapy

Radiotherapy, also called radiation therapy, is the treatment of cancer and other diseases with ionizing radiation. Ionizing radiation deposits energy that injures or destroys cells in the area being treated by damaging their genetic material, making it impossible for these cells to continue to grow. Although radiation damages both cancer cells and normal cells, the latter are able to repair themselves and function properly. Radiotherapy may be used to treat localized solid tumors, such as cancers of the skin, tongue, larynx, brain, breast, or cervix. It can also be used to treat leukemia and lymphoma (cancers of the blood-forming cells and lymphatic system, respectively).

Radiation therapy used according to the present invention may include, but is not limited to, the use of γ-rays, X-rays, and/or the directed delivery of radioisotopes to tumor cells. Other forms of DNA damaging factors are also contemplated such as microwaves and UV-irradiation. It is most likely that all of these factors effect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

Radiotherapy may comprise the use of radiolabeled antibodies to deliver doses of radiation directly to the cancer site (radioimmunotherapy). Antibodies are highly specific proteins that are made by the body in response to the presence of antigens (substances recognized as foreign by the immune system). Some tumor cells contain specific antigens that trigger the production of tumor-specific antibodies. Large quantities of these antibodies can be made in the laboratory and attached to radioactive substances (a process known as radiolabeling). Once injected into the body, the antibodies actively seek out the cancer cells, which are destroyed by the cell-killing (cytotoxic) action of the radiation. This approach can minimize the risk of radiation damage to healthy cells.

Conformal radiotherapy uses the same radiotherapy machine, a linear accelerator, as the normal radiotherapy treatment but metal blocks are placed in the path of the x-ray beam to alter its shape to match that of the cancer. This ensures that a higher radiation dose is given to the tumor. Healthy surrounding cells and nearby structures receive a lower dose of radiation, so the possibility of side effects is reduced. A device called a multi-leaf collimator has been developed and can be used as an alternative to the metal blocks. The multi-leaf collimator consists of a number of metal sheets which are fixed to the linear accelerator. Each layer can be adjusted so that the radiotherapy beams can be shaped to the treatment area without the need for metal blocks. Precise positioning of the radiotherapy machine is very important for conformal radiotherapy treatment and a special scanning machine may be used to check the position of your internal organs at the beginning of each treatment.

High-resolution intensity modulated radiotherapy also uses a multi-leaf collimator. During this treatment the layers of the multi-leaf collimator are moved while the treatment is being given. This method is likely to achieve even more precise shaping of the treatment beams and allows the dose of radiotherapy to be constant over the whole treatment area.

Although research studies have shown that conformal radiotherapy and intensity modulated radiotherapy may reduce the side effects of radiotherapy treatment, it is possible that shaping the treatment area so precisely could stop microscopic cancer cells just outside the treatment area being destroyed. This means that the risk of the cancer coming back in the future may be higher with these specialized radiotherapy techniques.

Stereotactic radiotherapy is used to treat brain tumours. This technique directs the radiotherapy from many different angles so that the dose going to the tumour is very high and the dose affecting surrounding healthy tissue is very low. Before treatment, several scans are analysed by computers to ensure that the radiotherapy is precisely targeted, and the patient's head is held still in a specially made frame while receiving radiotherapy. Several doses are given.

Stereotactic radio-surgery (gamma knife) for brain tumors does not use a knife, but very precisely targeted beams of gamma radiotherapy from hundreds of different angles. Only one session of radiotherapy, taking about four to five hours, is needed. For this treatment you will have a specially made metal frame attached to your head. Then several scans and x-rays are carried out to find the precise area where the treatment is needed. During the radiotherapy, the patient lies with their head in a large helmet, which has hundreds of holes in it to allow the radiotherapy beams through.

Scientists also are looking for ways to increase the effectiveness of radiation therapy. Two types of investigational drugs are being studied for their effect on cells undergoing radiation. Radiosensitizers make the tumor cells more likely to be damaged, and radioprotectors protect normal tissues from the effects of radiation. Hyperthermia, the use of heat, is also being studied for its effectiveness in sensitizing tissue to radiation.

C. Subsequent Surgery

Approximately 60% of persons with cancer will undergo surgery of some type, which includes preventative, diagnostic or staging, curative and palliative surgery. Curative surgery is a cancer treatment that may be used in conjunction with other therapies, such as the treatment of the present invention, chemotherapy, radiotherapy, hormonal therapy, gene therapy, immunotherapy and/or alternative therapies.

Curative surgery includes resection in which all or part of cancerous tissue is physically removed, excised, and/or destroyed. Tumor resection refers to physical removal of at least part of a tumor. In addition to tumor resection, treatment by surgery includes laser surgery, cryosurgery, electrosurgery, and miscopically controlled surgery (Mohs' surgery). It is further contemplated that the present invention may be used in conjunction with removal of superficial cancers, precancers, or incidental amounts of normal tissue.

Upon excision of part of all of cancerous cells, tissue, or tumor, a cavity may be formed in the body. Treatment may be accomplished by perfusion, direct injection or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

D. Gene Therapy

In another embodiment, the secondary treatment is a non-p53 gene therapy in which a second gene is administered to the subject. Delivery of a vector encoding p53 in conjunction with a second vector encoding one of the following gene products may be utilized. Alternatively, a single vector encoding both genes may be used. A variety of molecules are encompassed within this embodiment, some of which are described below.

1. Inhibitors of Cellular Proliferation

The tumor suppressor oncogenes function to inhibit excessive cellular proliferation. The inactivation of these genes destroys their inhibitory activity, resulting in unregulated proliferation. The tumor suppressors Rb, p53, p16, MDA-7, PTEN and C-CAM are specifically contemplated.

2. Regulators of Programmed Cell Death

Apoptosis, or programmed cell death, is an essential process for normal embryonic development, maintaining homeostasis in adult tissues, and suppressing carcinogenesis (Kerr et al., 1972). The Bcl-2 family of proteins and ICE-like proteases have been demonstrated to be important regulators and effectors of apoptosis in other systems. The Bcl-2 protein, discovered in association with follicular lymphoma, plays a prominent role in controlling apoptosis and enhancing cell survival in response to diverse apoptotic stimuli (Bakhshi et al., 1985; Cleary and Sklar, 1985; Cleary et al., 1986; Tsujimoto et al., 1985; Tsujimoto and Croce, 1986). The evolutionarily conserved Bcl-2 protein now is recognized to be a member of a family of related proteins, which can be categorized as death agonists or death antagonists.

Subsequent to its discovery, it was shown that Bcl-2 acts to suppress cell death triggered by a variety of stimuli. Also, it now is apparent that there is a family of Bcl-2 cell death regulatory proteins which share in common structural and sequence homologies. These different family members have been shown to either possess similar functions to Bcl-2 (e.g., BCl_{XL}, Bcl_{W}, Bcl_{S}, Mcl-1, A1, Bfl-1) or counteract Bcl-2 function and promote cell death (e.g., Bax, Bak, Bik, Bim, Bid, Bad, Harakiri).

### IX. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### Example 1

Methods. The inventor tested three groups of patients based on clinical and radiological data that were classified into non-cancer (Wong et al., 2000), granulomas (Cox et al., 2001) and cancer (Kuroki et al., 1993) groups. The manual method of FISH testing involves hybridizing three separate cytospin samples with the following probe combinations: (1) 3p22.1 and centromeric 3; (2) 10q22-23 and centromeric 10; and (3) centromeric 3, 7, 17 and 9p21.3

Scoring of probe sets. Probes 1 and 2 are scored by counting polysomies or monosomies of centromeric 3 and 10 and 3p22.1 and 10q22-23 and expressed as a ratio or deletions of 3p22.1 and 10q22-23 in 100 epithelial cells and 100 neutrophils. Probe 3 is a four-color FISH probe and abnormalities are expressed as monosomies or polysomies counted in 25 of the most atypical epithelial cells and 25 neutrophils. The total number of genetic aberrations from A+B in epithelial cells and neutrophils are scored.

Results. There were a total of 18 non-cancer patients and 21 cancer patients. Smoking status and other demographic characteristics are described in Table 1a and Table 1b. Patients without cancer (controls) had significantly higher pack years of smoking (p=0.026) than patients with cancer, even though there was no difference in age at time of sputum evaluation.

Patients with lung cancer had significantly more genetic aberrations for 3p22.1, 10q22-23, and Probe Set 3 in epithelial cells than non-cancer patients. (p=0.001, Table 1b).

In a univariate logistic regression model in estimating cancer status, we showed that cancer patients had marginally significant fewer years of smoking compared to non-cancer patients (Table 2), and that epithelial cell abnormalities, as well as epithelial cell and neutrophil abnormalities for all probes tested were significantly different between cancer and non-cancer status.

A multivariate logistic regression model in estimating cancer status showed that one unit increase in total genetic abnormality in epithelial cells increased the risk of developing cancer by 43%. Furthermore a one-unit increase in years of smoking decreased the risk of cancer by 8%.

TABLE 1(a)
Patients' Characteristics Grouped by Cancer Status

| | | Cancer Status | | |
|---|---|---|---|---|
| | | No | Yes | |
| Characteristics | | (n = 18) | (n = 21) | p-value* |
| Sex | | | | 0.75 |
| | Female | 9(50) | 12(57) | |
| | Male | 9(50) | 9(43) | |
| Ethnicity | | | | 0.21 |
| | Black | 2(11.1) | 0(0) | |
| | White | 16(88.9) | 21(100) | |
| Smoked before | | | | 0.60 |
| | yes | 17(94.4) | 15(88.2) | |
| | no | 1(5.6) | 2(11.8) | |
| Smoked in the | | | | 0.27 |
| last 12 months | | | | |
| | yes | 7(41.1) | 3(20.0) | |
| | no | 10(58.8) | 12(80.0) | |
| Smoking status | | | | 0.56 |
| | Never | 1(5.6) | 2(11.8) | |
| | Former | 7(38.9) | 9(52.9) | |
| | Recently | 3(16.7) | 3(17.7) | |
| | quitter | | | |
| | Current | 7(38.9) | 3(17.7) | |

*Fisher's exact test

TABLE 1(b)
Patients' Characteristics Grouped by Cancer Status

| | Cancer | | | | | | | |
|---|---|---|---|---|---|---|---|---|
| Characteristics | Status | N | mean | std | min | median | max | p-value* |
| Age | | | | | | | | 0.69 |
| | No | 18 | 64.11 | 5.07 | 56 | 63.5 | 74 | |
| | Yes | 20 | 64.05 | 8.4 | 50 | 63 | 79 | |
| Years of smoking | | | | | | | | 0.039 |
| | No | 18 | 38.22 | 13.37 | 0 | 39.5 | 55 | |
| | Yes | 17 | 29.18 | 14.31 | 0 | 34 | 48 | |
| Number of cigarettes | | | | | | | | 0.18 |
| | No | 18 | 26.06 | 12.9 | 0 | 20 | 50 | |
| | Yes | 17 | 25.47 | 27.28 | 0 | 20 | 120 | |
| Pack-year | | | | | | | | 0.026 |
| | No | 18 | 50.29 | 23.92 | 0 | 45.5 | 110 | |
| | Yes | 17 | 41.04 | 47.52 | 0 | 35 | 210 | |
| Epithelia 3p + 10q | | | | | | | | 0.17 |
| | No | 17 | 4.65 | 2.8 | 0 | 5 | 9 | |
| | Yes | 20 | 7.15 | 4.43 | 2 | 6 | 15 | |
| Epithelia A + B + | | | | | | | | 0.003 |
| C + D | | | | | | | | |
| | No | 18 | 2.78 | 1.8 | 0 | 3 | 6 | |
| | Yes | 21 | 7.38 | 4.83 | 1 | 7 | 20 | |
| Epithelia All | | | | | | | | 0.001 |
| | No | 17 | 7.47 | 3.45 | 0 | 7 | 13 | |
| | Yes | 20 | 14.75 | 6.82 | 7 | 13.5 | 34 | |
| Neutrophils 3p + 10q | | | | | | | | 0.81 |
| | No | 7 | 4.86 | 3.18 | 2 | 4 | 10 | |
| | Yes | 15 | 4.4 | 3.42 | 0 | 4 | 14 | |
| Neutrophils A + | | | | | | | | 0.13 |
| B + C + D | | | | | | | | |
| | No | 17 | 2.41 | 2.55 | 0 | 2 | 9 | |
| | Yes | 21 | 3.62 | 2.75 | 0 | 3 | 9 | |
| Neutrophils All | | | | | | | | 0.73 |
| | No | 7 | 7.14 | 5.3 | 2 | 4 | 16 | |
| | Yes | 15 | 7.67 | 4.64 | 1 | 7 | 19 | |
| 3p + 10q | | | | | | | | 0.55 |
| | No | 7 | 7.86 | 4.38 | 2 | 8.0 | 15 | |
| | Yes | 14 | 10.21 | 5.82 | 4 | 7.5 | 21 | |
| A + B + C + D | | | | | | | | 0.003 |
| | No | 17 | 5.24 | 3.11 | 2 | 5.0 | 12 | |
| | Yes | 21 | 11.00 | 6.24 | 2 | 10.0 | 27 | |
| 3p + 10q + A + | | | | | | | | 0.070 |
| B + C + D | | | | | | | | |
| | No | 7 | 13.43 | 5.59 | 6 | 14.0 | 20 | |
| | Yes | 14 | 21.21 | 8.61 | 13 | 17.5 | 40 | |

*Wilcoxon rank sum test
Epithelia 3p + 10q: Deletions of 3p + 10q in epithelia cells
Epithelia A + B + C + D: The sum abnormalities (deletions) for probes A, B, C, D in epithelia cells
Epithelia All: The sum of all abnormalities for 3p, 10q, A, B, C, D in epithelia cells
Neutrophils 3p + 10q: Deletions of 3p + 10q in neutrophils cells
Neutrophils A + B + C + D: The sum of abnormalities (deletions) for the probes A, B, C, D in neutrophils cells
Neutrophils All: The sum of all abnormalities for 3p, 10q, A, B, C, D in neutrophils cells
3p + 10q: Deletions of 3p + 10q in epithelia and neutrophils cells
A + B + C + D: The sum of abnormalities (deletions) for the probes A, B, C, D in epithelia and neutrophils cells
3p + 10q + A + B + C + D: The sum of all abnormalities for 3p, 10q, A, B, C, D in epithelia and neutrophils cells

TABLE 2
Univariate Logistic Regression Model in Estimating
Cancer Status (Cancer vs. no Cancer)

| | Parameter | Standard | | Odds |
|---|---|---|---|---|
| Characteristics | Estimate | Error | P-value | Ratio |
| Age | −0.001 | 0.05 | 0.98 | 0.999 |
| Years of smoking | −0.05 | 0.03 | 0.077 | 0.95 |
| Number of cigarettes | −0.001 | 0.02 | 0.93 | 0.999 |
| Pack-year | −0.01 | 0.01 | 0.47 | 0.99 |
| Epithelia 3p + 10q | 0.19 | 0.10 | 0.065 | 1.21 |
| Epithelia A + B + C + D | 0.45 | 0.17 | 0.006 | 1.57 |
| Epithelia All | 0.38 | 0.13 | 0.004 | 1.46 |
| Neutrophils 3p + 10q | −0.04 | 0.14 | 0.76 | 0.96 |
| Neutrophils A + B + C + D | 0.18 | 0.14 | 0.18 | 1.20 |
| Neutrophils All | 0.02 | 0.10 | 0.81 | 1.03 |
| 3p + 10q | 0.10 | 0.10 | 0.35 | 1.10 |
| A + B + C + D | 0.30 | 0.11 | 0.006 | 1.35 |
| 3p + 10q + A + B + C + D | 0.23 | 0.13 | 0.079 | 1.25 |

TABLE 3
Multivariate Logistic Regression Model in Estimating
Cancer Status (Cancer vs. no Cancer)

| | Parameter | Standard | | |
|---|---|---|---|---|
| Characteristics | Estimate | Error | P-value | Odds Ratio |
| Years of smoking | −0.07 | 0.04 | 0.041 | 0.93 (0.87, 0.997) |
| Epithelia All | 0.36 | 0.15 | 0.015 | 1.43 (1.07, 1.92) |

Summary. There is a marked predisposition to develop lung cancer based on this test of genetic instability by FISH with cancer cases having significantly higher levels of genetic aberrations in epithelial cells than non-cancer patients. Differentiation between granulomas and cancer patients may be made on the basis of differences in genetic instability for centromeric 3 and 17 in neutrophils.

Both cancer patients and patients with granulomas had similar genetic aberrations for chromosomes 3p and 10q in the epithelial cells and neutrophils in the sputum. However there appeared to be significant differences for certain chromosomes in epithelial cells versus neutrophils in cancer patients compared to patients with granulomatous disease. These findings suggest that the pathogenesis of lung cancer and granulomatous disease involves an early stem cell that gives rise to both lung cancer and granulomas, however at an early stage there is divergence of genetic alterations.

Both conditions may involve circulating stem cells with different genetic phenotypes (cancer stem cells lack centromeric 17 abnormalities) that under the influence of GM-CSF either locally in the lung or in the bone marrow can differentiate into epithelial cells, cells of the myeloid cells including neutrophils and macrophages that are predestined to result in carcinoma or granulomas.

### Example 2

### Prognostic Value of Surfactant Protein A Gene Deletion for Patients with Stage I Non-Small Cell Lung Cancer

Materials—Tissue Samples. A total of 505 consecutive patients with stage 1 NSCLC underwent definitive surgical resection, defined as a lobectomy or a pneumonectomy, from 1986 to 1996 at The University of Texas MD Anderson Cancer Center. Tissue samples from 130 patients from this group were found to contain an adequate number of both tumor cells and cells from adjacent normal bronchiole epithelium and were included in this study. Follow-up information was obtained from chart reviews and reports from the institution's tumor registry service. Lung tissues from 63 patients who were treated at this institution during the same period but who were not diagnosed with lung cancer were obtained and used as controls. The study design was reviewed and approved by the institution's surveillance committee. No patients received adjuvant chemotherapy or radiation therapy before or after surgery. Tissue sections (4 mm thick) were obtained from each tissue block, stained with hematoxylin-eosin, and re-reviewed independently by at least one pathologists (R. L. K and N. P. C) to confirm the diagnosis and the presence of both tumor and adjacent bronchial epithelium. Histologic type of the tumors was determined according to the WHO classification. Areas of interest were marked for future micro-dissection.

Materials—Specific SP-A Probe. A BAC clone containing SP-A genomic sequence was selected from a BAC-PAC contig (kindly provided by Dr. Peter A. Steck, Department of Neuro-Oncology, M.D. Anderson Cancer Center) in chromosome 10q22-24. To confirm whether genomic sequences of both SP-A1 and SP-A2 are located in the clone and size of the clone, the clone was checked by means of a PCR-based screening protocol using specific primers for SP-A1 (ACACCAACTGGTACCGACC (SEQ ID NO:13), TGGAGCCTCAGGATGGAGG (SEQ ID NO: 14 and SP-A2 CTGCTGATGAACAAATCTGCA (SEQ ID NO: 15), GTGGCACATGGTATGTGCTC (SEQ ID NO:16)), followed by sequencing of both ends of the clone and running the sequences in BlastN in the Celera database (myscience.appliedbiosystems.com). The chromosomal location of the clone was confirmed on a normal metaphase spread in combination with centromeric probe for chromosome 10, CEP 10 (Vysis, Downers Grove, Ill.).

Methods—Specimen Preparation and FISH. To isolate whole nuclei from tumor cells and adjacent bronchial tissue cells, representative tumor and adjacent bronchi were first located microscopically and then marked on hematoxylin-eosin-stained sections. Corresponding areas were then identified on the paraffin blocks, and core tissue samples of 1.0 mm in diameter were taken from each block using a 22-gauge needle (BIOPUNCH, Buffalo, N.Y.). The same procedure was used to sample control lung tissues and their adjacent bronchi. To isolate cell nuclei from the specimens, the paraffin-embedded tissue samples were deparafinized by immersing in xylene for 1 h, followed by immersing in a series of dilution of ethanol (100%, 90%, and 70% for 10 min each). After being transferred into distilled water, the specimens were incubated with 0.1% protease (Sigma P8038, Sigma Chemical Co., St. Louis, Mo.) at 37° C. for 10 min. Cytospins preparation were generated on glass slides using a SHANDON-Cytospin 2 cytocentrifuge (Pittsburgh, Pa.) and then fixed with Carnoy's solution for 20 min. Dual-color FISH was performed as described previously using the SP-A probe which was digoxigenin-labeled and the internal reference, a centromeric probe to chromosome 10, which was directly fluorescence labeled in spectrum orange. One hundred nanograms of each probe was mixed with a 30-fold excess of human Cot-1 DNA (Life Technologies, Rockville, Md.) in 10 ul of LSI hybridization buffer (Vysis) and mounted on a slide. Hybridization was performed by incubation the slides at 37° C. overnight. Signals were detected for SP-A gene using antidigoxigenin-FITC (Roche Diagnostics Corporation, Indianapolis, Ind.). Nuclei were counterstained with DAPI (Vysis).

Methods—Scoring FISH Signals. Slides were examined using microscopes equipped with appropriate filter sets (Leica Microsystems, In., Buffalo, N.Y.). Two hundred cells were counted on each slide. To ensure random analysis of the cells, at least two areas were examined on each slide. Overlapping cells and cells that had indistinct or blurry signals were not scored, and care was taken not to interpret a split signal as two signals. More and fewer signals from the SP-A probe than from the CEP 10 probe indicated SP-A gene gain and loss, respectively. FISH signaling patterns of SP-A were defined as abnormal if there were more or fewer copies of the SP-A signals than of CEP 10 signals, beyond a certain cutoff (baseline value). The cutoff value was calculated from normal tissue samples and was defined as the mean number of cells having an abnormal SP-A signal pattern on FISH analysis plus three standard deviations.

Methods—Statistical Analysis. All statistical analyses were performed using SAS software (version 6.12: SAS Institute Inc., Cary, N.C.). Overall, disease specific, and disease-free survival rates were calculated using the Kaplan-Meier method. All survival times were calculated from the date of surgery. Patients were stratified into two groups based on time to relapse from date of surgery. Early relapse was defined as relapse within 24 months from the date of surgery, and late relapse was defined as relapse after 24 months from the date of surgery. Disease specific survival time was calculated from the date of surgery to date of death from cancer related cause. The Chi-square test was used to evaluate associations between categorical variables. The Mann-Whitney rank test was used to assess differences in continious variables by dichotomous relapse status. The univariate Cox proportional hazards model was used to evaluate the unadjusted association between survival time and various clinically and histopathologically interesting risk factors (age, sex, race, smoking history, alcohol consumption, histological subtype, and TNM stage). Multivariate Cox models were used to model the increased risk associated with increases in SP-A genomic copy number on survival time, after adjusting for the same clinical and histopathological parameters mentioned above. Tukey's HSD test was also used to analyze the relationship between relapse and risk factors. All p-values were determined by two-sided tests. P values less than 0.05 were considered statistically significant.

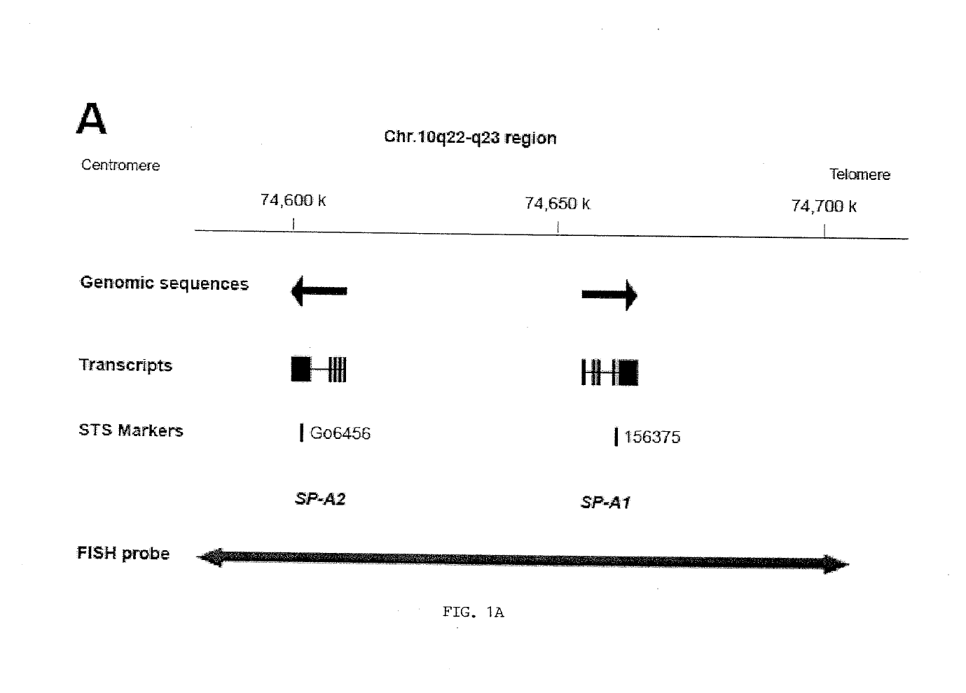

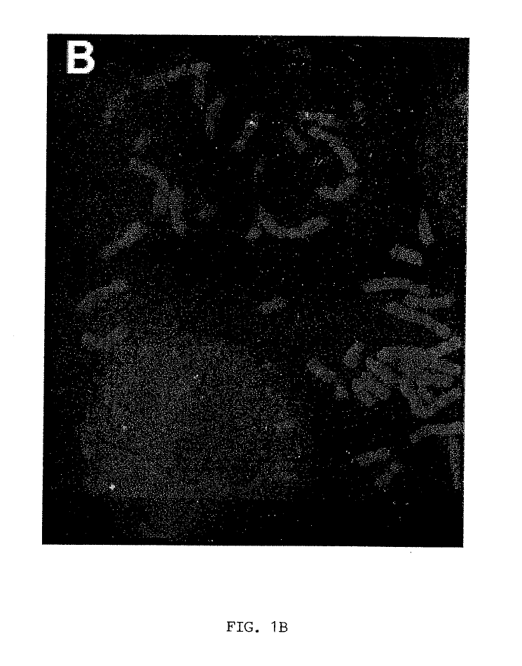

Results. One BAC clone of approximately 110 kb was mapped by PCR to cover both SP-A1 and SP-A2 (FIG. 1A). Two green signals (corresponding to the SP-A probe) and two orange signals (corresponding to the CEP 10) were detected in the control interphases and metaphases from the normal lymphocytes (FIG. 1B). Eighty-nine percent of NCI-H358 lung cancer cells, which have homozygous deletion of the SP-A genome, had fewer SP-A signals than CEP 10 signals.

Table 4 summarizes the patient and tumor characteristics for the 130 patients who had adequate tumor and bronchial specimens for study. Patient ages raged from 39 to 84 years, with a median age of 64.5±9.5 years. The cancers were of the following histological subtypes: SCC (n=59), adenocarcinoma (n=54), bronchioalveolar carcinoma (n=8), large cell carcinoma (n=4), adenosquamous carcinoma (n=3), and unclassified cases (n=2). Lung cancers were further characterized as early relapsing (relapse within 24 months of surgery) and late relapse or in long-term remission (relapse more than 24 months of surgery or no relapse with follow-up of more 24 months). Thirty-nine of the patients with disease relapsed and 31 of them had early relapses after surgery. The median follow-up duration was 60 months (range: 6-108 months).

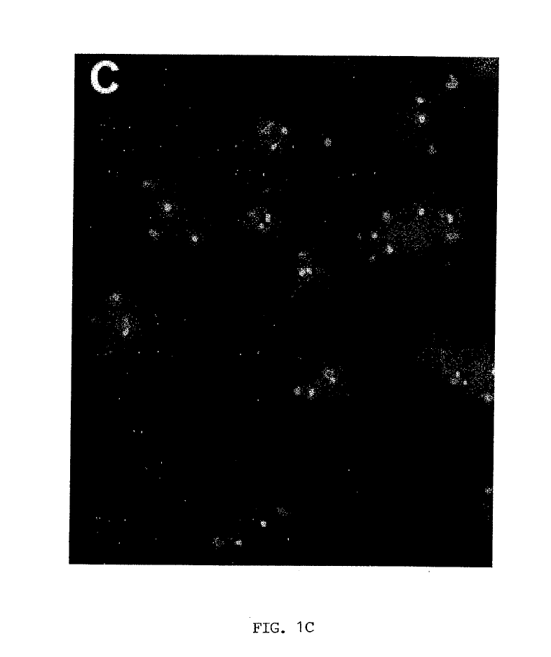

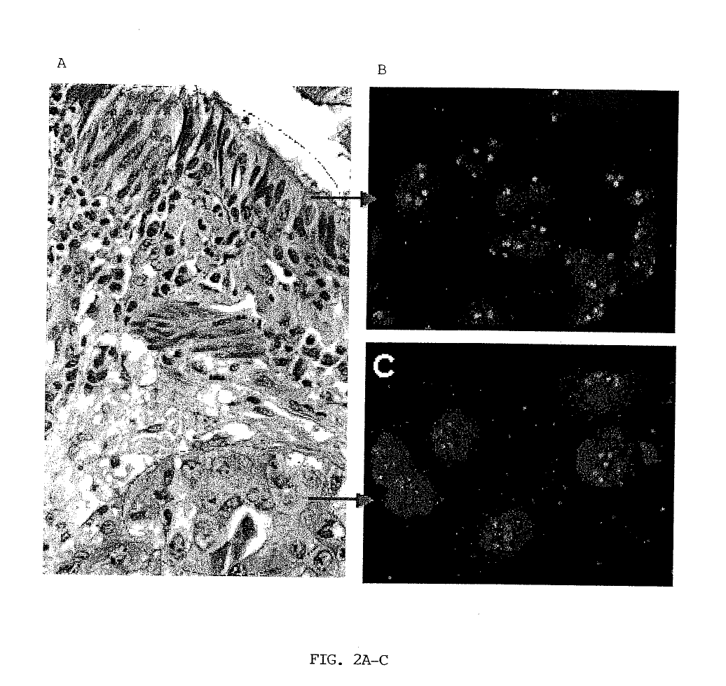

Dual color FISH with the SP-A specific probe and CEP10 was used to determine the frequency of genomic alterations of SP-A in the NSCLC specimens. Control hybridization values were established for the probes by using nuclei extracted from 63 normal lung tissues as targets. FISH analysis was successful for all the specimens. In normal cells, the percentage of nuclei found to have a gain or loss of SP-A signals ranged between 0% and 2%. Therefore, a specimen with a SP-A deletion was defined when ≧4% of cells show deletion of SP-A. Positive cases with SP-A deletion were found in tumor of all the histological subtypes tested. SP-A deletion was found in 87% of all 130 tumors and in 32% of their adjacent bronchioles (FIGS. 2A and 2B). The mean rate of SP-A deletion from the bronchial epithelial cells adjacent to the tumors was 12.29%±4.28%. Deletion of SP-A occurred to a much higher degree in carcinoma cells (38.28%±5.08%). There was a statistically significant correlation between deletions of SP-A in tumor and deletion in adjacent normal-appearing bronchial epithelial cells (P<0.001).

The inventors tested for correlations between the status of SP-A genetic copy numbers in the tumors or adjacent bronchi and various clinicopathological features. There were no statistical significant correlations between frequency of SP-A deletion and patient age, or sex, or tumor histological type, or TNM stage (T1N0M0 versus T2N0M0). However, deletion of SP-A occurred more frequently in the tumors and normal-appearing bronchial epithelium of the patients who smoked than in those of patients who had never smoked (P<0.001 for both). Using the univariate Cox proportional-hazards model to assess the variants' effects on time to relapse, it was found that only SP-A deletions in the tumor and adjacent normal-appearing bronchial tissues significantly increased risk of the relapse (P=0.035, P<0.0001, respectively) (Table 5). This result was confirmed using the HSD mean-rank test, which showed that SP-A deletions in both tumors and their adjacent bronchial tissues were strongly correlated with relapse (P<0.001 for both).

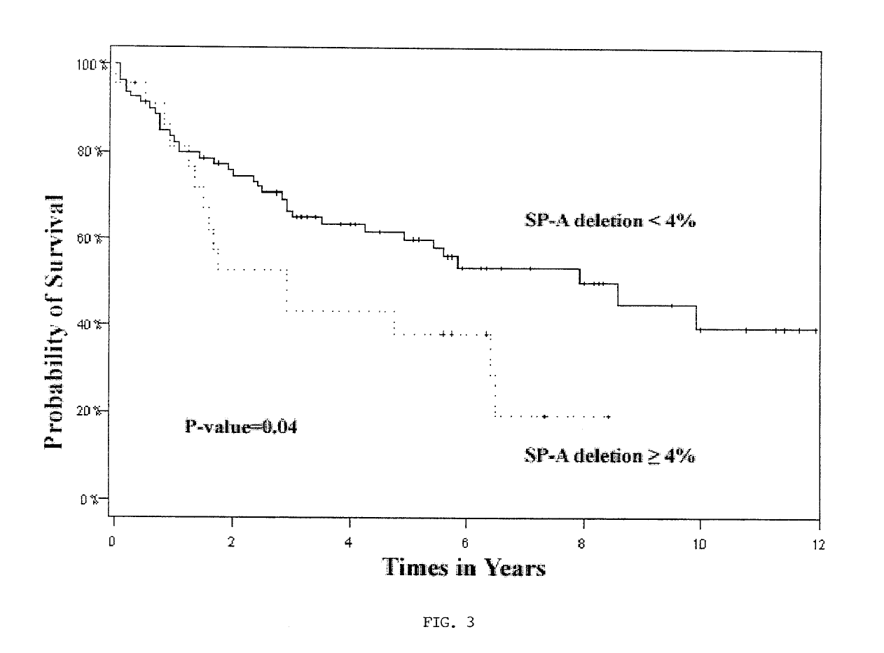

To determine whether SP-A deletion can predict survival time for patients with pathologic stage I NSCLC, a univariate analysis was first performed using the Cox model. The inventors found that only SP-A deletion in adjacent normal-appearing bronchioles was a predictive of shorter survival time versus no SP-A deletions in the adjacent bronchi (P=0.026) (Table 6). Interestingly, patient age may turn out to be another predictive factor for survival, because older patients showed marginally short overall survival (P=0.037). However, when stepwise selection was used to choose the appropriate multivariate model, a one-unit (1%) increase in SP-A deletion in adjacent bronchiole epithelial cells increased the risk of dying by 19%, whereas a one-unit increase in age increased the risk of dying by only 4%. There seems to be some evidence that having such a high deletion rate in adjacent bronchial epithelial cells results in shorter survival. This finding was supported by the Kaplan-Meier Curve analysis of probability of lung cancer-specific survival for patients with SP-A deletion versus patients without SP-A deletion in adjacent bronchial epithelial cells (FIG. 3). The site of disease recurrence was available for a subset of the clinical specimens (N=39). The average SP-A deletion level was highest in those patients who subsequently developed contralateral/distant metastases including 6 brain metastases. In contrast, SP-A deletion level was lowest in patients whose metastases were restricted to the ipsilateral lung. There is a significant difference between deletions of SP-A in those patients who progressed to contralateral/distant metastases and SP-A deletion in patients who progressed to have metastases restricted to the ipsilateral lung (P=0.023). All together, the data suggest that the level of SP-A deletion from cells in the bronchial epithelial tissue adjacent to tumor is the most significant prognostic indicator for disease-specific survival.

TABLE 4
Patient and disease characteristics associated with the
archived, formalin-fixed, paraffin-embedded tissue*

| Covariate | No. patients (%) | |
|---|---|---|
| Race | | |
| Asian | 1 | (0.8) |
| Black | 9 | (6.9) |
| Hispanic | 8 | (6.2) |
| White | 112 | (86.2) |
| Sex | | |
| Female | 62 | (47.7) |
| Male | 68 | (52.3) |
| Smoking status | | |
| Yes | 118 | (92.9) |
| No | 9 | (7.1) |
| Alcohol consumption | | |
| <1 drink/day | 59 | (45.7) |
| ≧1 drink/day | 70 | (54.3) |
| Tumor histology | | |
| SCC | 59 | (45.3) |
| Adenocarcinoma | 54 | (41.5) |
| Other | 17 | (13.2) |
| TNM stage | | |
| T1N0M0 | 63 | (48) |
| T2N0M0 | 67 | (52) |
| Relapses^{†} | | |
| Early relapse (≦24 mo) | 31 | (79.4) |
| Long-term relapse (>24 mo) | 7 | (20.6) |

*Data was from 130 patients with evaluable tumors and adjacent bronchial specimens in the tumor blocks, unless otherwise indicated.
^{†}Data was from 39 patients who had relapses during the study period.

TABLE 5
Univariate Cox proportional hazards model estimates
of time to relapse in the 130 evaluable patients

| | Parameter estimate | | Relative |
|---|---|---|---|
| Covariate | (mean ± S.D.) | P value* | risk |
| Age | 0.030 ± 0.020 | 0.072 | 1.030 |
| Gender (female | −0.460 ± 0.340 | 0.170 | 0.630 |
| vs. male) | | | |
| Smoking status | 0.990 ± 1.010 | 0.330 | 2.690 |
| (yes vs. no) | | | |
| Alcohol (≧1 drink/day | −0.100 ± 0.330 | 0.760 | 0.900 |
| vs. <1 drink/day) | | | |
| TNM stage (T1N0M0 | −0.00030 ± 0.001 | 0.820 | 0.99970 |
| vs. T2N0M0) | | | |
| SP-A deletions | 0.010 ± 0.010 | <0.00001 | 1.190 |
| in adjacent | | | |
| bronchial tissue | | | |
| SP-A deletions | 0.170 ± 0.040 | 0.0350 | 1.010 |
| in tumors | | | |

Abbreviations: S.D., standard deviation; SP-A, surfactant protein A.
*All P values were determined by two-sided tests. P values less than 0.05 were considered statistically significant (indicated in bold).

TABLE 6
Univariate Cox proportional hazards model estimates of
time to survival of the 130 evaluable cancer patients

| | Parameter estimate | | Relative |
|---|---|---|---|
| Covariate | (mean ± S.D.) | P value* | risk |
| Age | 0.030 ± 0.010 | 0.037 | 1.040 |
| Gender (female | 0.130 ± 0.280 | 0.640 | 0.880 |
| vs. male) | | | |
| Smoking status | 0.450 ± 0.530 | 0.390 | 0.640 |
| (yes vs. no) | | | |
| Alcohol (≧1 drink/day | −0.280 ± 0.280 | 0.310 | 0.760 |
| vs. <1 drink/day) | | | |
| TNM stage (T1N0M0 | −0.0004 ± 0.0006 | 0.510 | 1.0004 |
| vs. T2N0M0) | | | |
| SP-A deletions | 0.090 ± 0.040 | 0.026 | 1.090 |
| in adjacent | | | |
| bronchial tissue | | | |
| SP-A deletions | 0.010 ± 0.010 | 0.530 | 1.010 |
| in tumors | | | |

Abbreviations: S.D., standard deviation; SP-A, surfactant protein A.
*All p-values were determined by two-sided tests. P-values less than 0.05 were considered statistically significant (indicated in bold).

Discussion. SP-A alterations have been studied in human lung cancers. O'Reilly et al. first observed synthesis of SP-A in a cell line derived from a lung adenocarcinoma using immunoblot analysis and ELISA (O'Reilly et al., 1988). Linnoila et al. found a high level of SP-A expression in adenocarcinomas by immunohistochemical analysis, particularly in those found to have a papillolepidic growth pattern (Linnoila et al., 1992). Others using RT-PCR and RNA in situ hybridization techniques confirmed the results (Betz et al., 1995; Kuroki et al., 1993; Broers et al., 1992; O'Reilly et al., 1988; Linnoila et al., 1992). Shijubo et al. found that 27 of 67 patients with lung adenocarcinomas had high levels of SP-A in their pleural effusions, while patients with other histologic types of lung cancers, adenocarcinomas originating from different primary sites, and tuberculosis had low levels of SP-A in their pleural effusions, suggesting that detection of SP-A in malignant effusions might help distinguish primary lung adenocarcinoma form other adenocarcinomas of miscellaneous origin (Shijubo et al., 1995; Shijubo et al., 1992).

Fujita et al. however, using both immunohistochemical analysis and RT-PCR, dectected SP-A expression in only one of 16 lung cancer cell lines, six of which were adenocarcinoma (Fujita et al., 2003). Furthermore, Zamecnik et al. observed significantly less of SP-A immunoreactivity in poorly differentiated lung adenocarcinomas than in well differentiated forms (Zamecnik et al., 2002). Tsutsumida et al. recently reported that a high MUC1/SP-A ratio could predict the recurrence and outcome of patients with pulmonary adenocarcinomas (Tsutsumida et al., 2004). The inventors consider that such inconsistent results might be attributable to (a) the different techniques used to detect SP-A aberrations or (b) the small sample sizes and mixed stages of the tumors studied. Furthermore, no previous study has evaluated the prognostic value of SP-A in stage I NSCLC. To address these issues, the inventors chose to use FISH to measure the SP-A genetic copy numbers in a large population of patients with stage I NSCLC for whom complete follow-up information was available.

The use of FISH for diagnostic purposes has increased significantly in the last few years, primarily because FISH permits visualization and examination of genetic aberrations in a large number of cells and because it can be easily applied to both archival and fresh material (Jiang and Katz, 2002). One example of use FISH in diagnosis in its use with the Her-2/neu genetic probe to guide the post-resection of breast cancer (Harbeck et al., 1999; Winston et al., 2004; Bartlett et al., 2001; Pauletti et al., 2000). This methodology has become the standard of care and its results have been proven to predict outcomes better than those of immunohistochemical assay for protein expression (Winston et al., 2004). Despite such successes, two limitations of the FISH technique inhibit its application as a diagnostic tool. First, all current conventional FISH locus-specific probes are made from large genomic fragments. The probes usually contain genomic sequences coding for several genes, and therefore, are too large to detect abnormalities of a single gene that are associated with cancer progression and always result in high rates of false-negative or false-positive findings. Second, FISH paraffin sections frequently displays artifacts from auto-fluorescence, incomplete sectioning of nuclei, and high background signaling which makes it extremely difficult to interpret FISH results reliably. To overcome these obstacles in the current study, a DNA probe was developed that specifically covers the full length of the genomic coding region for SP-A and then applied the probe to isolated cell nuclei that had been micro-dissected form whole tumors and adjacent bronchial tissues. This approach produced bright, distinct, and non-truncated signals when the samples were probed. Therefore, the result obtained by these probes reflected the true aberrations of the SP-A gene in the specimens tested.

The major value of prognostic markers is to identify patients at a high risk for cancer-related events, such as recurrence or survival, and to determine the need for and effects of adjuvant therapy. To the inventors' knowledge, this study is the first to investigate the prognostic importance genetic aberrations of SP-A in NSCLC. Our findings from a large retrospective study demonstrate that SP-A deletion is a statistically significant predictor of poor outcome in patients with early stage NSCLC. Furthermore, we found that SP-A deletions occur with equal frequency among all histologic types of NSCLC and are associated with tobacco use. These findings might contribute to the development of a model for the molecular classification of lung cancer. More important, they suggest that the SP-A gene copy number could be an ideal marker for assessing the prognosis of patients with early stage NSCLC and evaluating the effects of chemotherapeutic agents.

Most known prognostic biomarkers are present within the lung tumor itself, but the methods used to detect them are invasive. Bronchial brushing and sputum specimens can provide bronchial epithelial cells from distinct areas of the airway, and these cells are suitable for examination for various morphological features and biomarkers. In addition, collection of these cells is less expensive and invasive than surgical removal of tissue, and therefore, can be performed routinely. If genetic aberrations in the bronchial epithelium reflect lung cancer development and can predict patient prognosis, patients can be monitored by testing for genetic changes in bronchial brushing or even sputum specimens containing exfoliated bronchial epithelial cells.

The most striking finding in the present study was that SP-A deletion in the normal appearing bronchial tissue adjacent to tumor was closely related to SP-A deletion in the tumor cells and was strongly correlated with patient survival and disease relapse. The finding may have clinical important because the SP-A probe might be useful as surrogate biomarkers of recurrent disease and because this FISH analysis might be a reliable means of testing for SP-A deletions in cells from bronchial brushings or sputum. For example, patients with persistent high levels of SP-A gene deletion in their bronchial brushings or sputum cells might benefit from adjuvant therapy.

The SP-A deletions were observed in both normal-appearing adjacent bronchial tissues and, to a higher degree, in tumor cells. Non-cancerous tissue, although it appears to be histologically normal, might be abnormal because environmental factors such as exposure to cigarette smoke and have molecular events related to lung carcinogenesis. Indeed, the SP-A deletion status of both lung tumors and their adjacent bronchial tissues are closely associated with tobacco use. Deletion of SP-A occurs more frequently in the bronchial epithelium of smoking patients than in the bronchial epithelium of the smoking patients than in the bronchial epithelium of non-smoking patients. These results suggest that development of the SP-A genetic aberrations might be part of a multi-step carcinogenic process caused by chronic exposure to tobacco smoke and that SP-A deletion might be a tumor specific phenomenon in tobacco related lung cancer, although further studies are required to investigate the effect of SP-A genomic aberration at the transcriptional and protein levels, and mutation or mythylation status of the SP-A in lung cancer. In addition, one of the more intriguing outcomes of this study is a demonstration of differential SP-A deletion in ipsilateral lung versus contralateral lung/distant metastases. An analysis of deletions of SP-A in adjacent normal-appearing bronchial epithelial cells suggested an interesting implication for interpreting the results of SP-A deletion disease recurrence. Thus, high levels of SP-A deletion appear to identify patients who develop metastases outside ipsilateral lung and also predict the recurrence of those metastases.

In conclusion, based on information for a large population of patients with stage I NSCLC, the results reported here indicate that SP-A deletion is associated with malignant tumor progression and smoking, thereby making it a potentially suitable prognostic marker. In the case of early stage NSCLC, determination of SP-A genetic copy number using FISH might be useful in distinguishing patients with poor prognosis and guiding the regimens of adjuvant chemotherapy for patients. Confirmatory results from studies by independent groups and double-blinded, prospective analysis of the use of this maker are necessary to validate the current findings.

### Example 3

### Predictions of Patients' Cancer Status Using a Combination of Probes

The primary objective of the study was to identify a set of probes and image analysis parameters which can separate the benign cases from the malignant groups. A total of 61 sputum cases were examined. Twenty-eight were benign cases and the rest were malignant tumors. Four patients were deleted from the study due to uncertainty concerning their disease status. Three additional patients were deleted due to missing information of image analysis parameters. Therefore, there were 28 maligant cases and 26 benign cases in the analysis dataset. Eight “in-house” probes were used: Epithelial cep10 10q deletion, Epithelial cep10 polisomies, Epithelial cep3 3p deletion, Epithelial cep3 polisomies, Neutrophils cep10 10q deletion, Neutrophils cep10 polisomies, Neutrophils cep3 3p deletion, Neutrophils cep3 polisomies. Furthermore, commercially available probes (Neutrophils A, B, C, D and Epithelial A, B, C, D) were also analyzed. Patients' characteristics such as age, gender and smoking status are summarized using descriptive statistics.

The main findings can be summarized as the follows. First, patients' cancer status was significantly associated with some probes and image analysis parameters according to Wilcoxon rank sum test. They are Epithelial cep 3 3p deletion, Epithelial cep 3 polisomies, Epithelial A, Neutrophils cep10 10q deletion, Neutrophils cep 10 polisomies, SDV I.O.D., SDV M.O.D., and SDV St. Dev. Patients' age and gender information were well balanced between cancer patients and non-cancer patients. There was a trend that non-cancer patients smoked more than cancer patients. Second, for cancer patients, they were regrouped into adenocarcinoma and non-adenocarcinoma. The Neutrophils A levels were significantly lower in patients with adenocarcinoma than other cancer patients (p-value: 0.049). The other probe measurements were not statistically significantly different between the two groups. And third, three multivariate logistic regression models were identified to predict cancer status. Model A is a prediction model based on the probes alone. The probes utilized in Model A are Epithelial cep 10 10q deletion, Epithelial cep 10 polisomies, Epithelial cep 3 3p deletion, Epithelial A, Neutrophils cep10 10q deletion. Model B is a prediction model based only on the image analysis parameters, which includes SDV I.O.D., SDV image, SDV M.O.D. Model C is a prediction model which combines the probes and image analysis parameters. Parameters included in Model C are Epithelial cep 10 polisomies, Epithelial cep 3 3p deletion, Neutrophils cep 10 10q deletion, SDV I.O.D, and SDV image. Model C achieves 92% and 81% sensitivity and specificity.

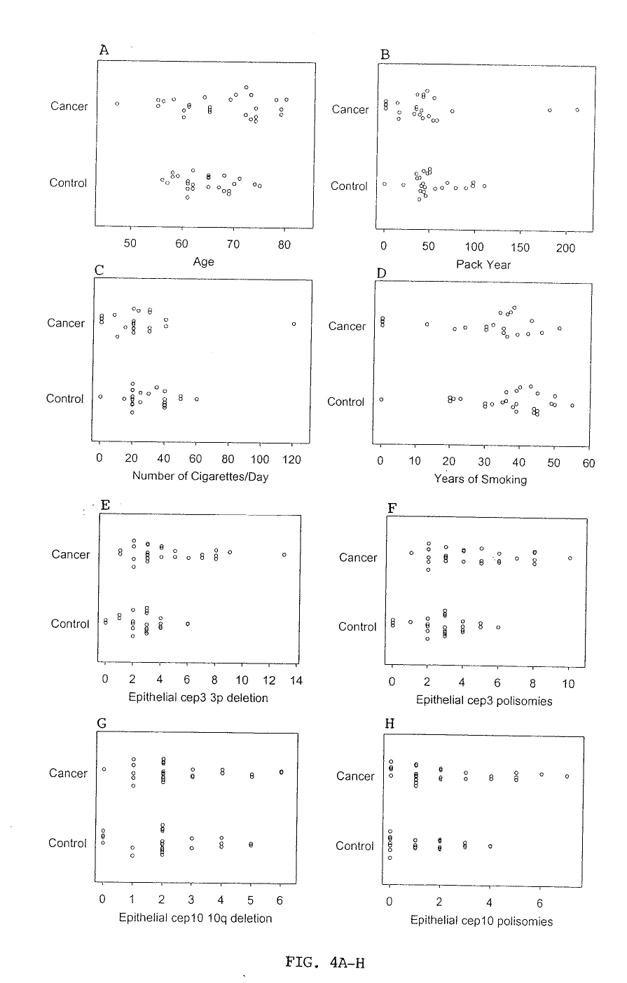

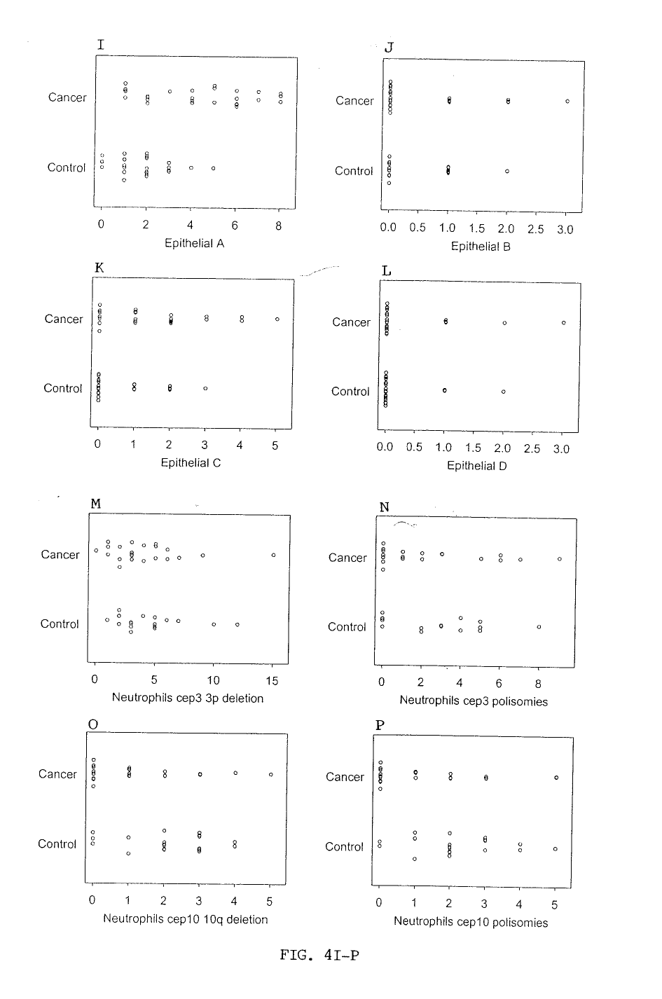

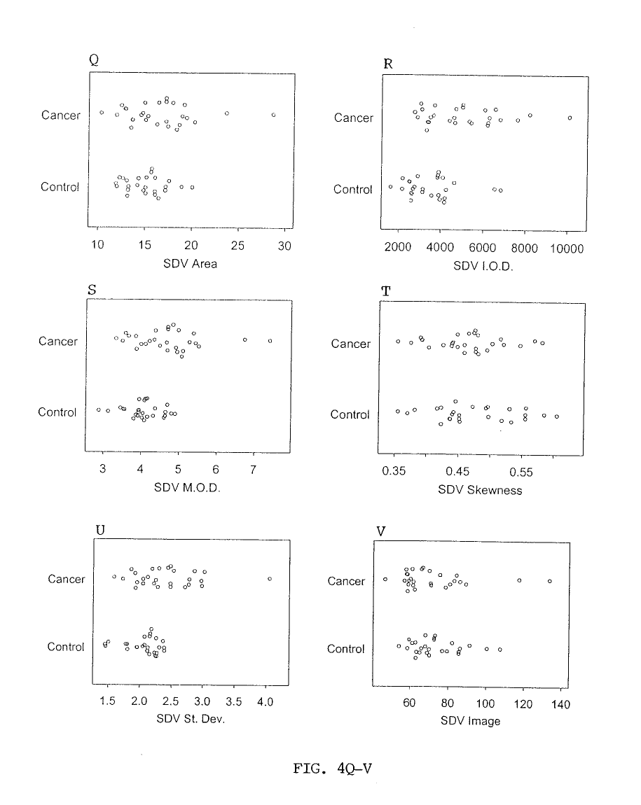

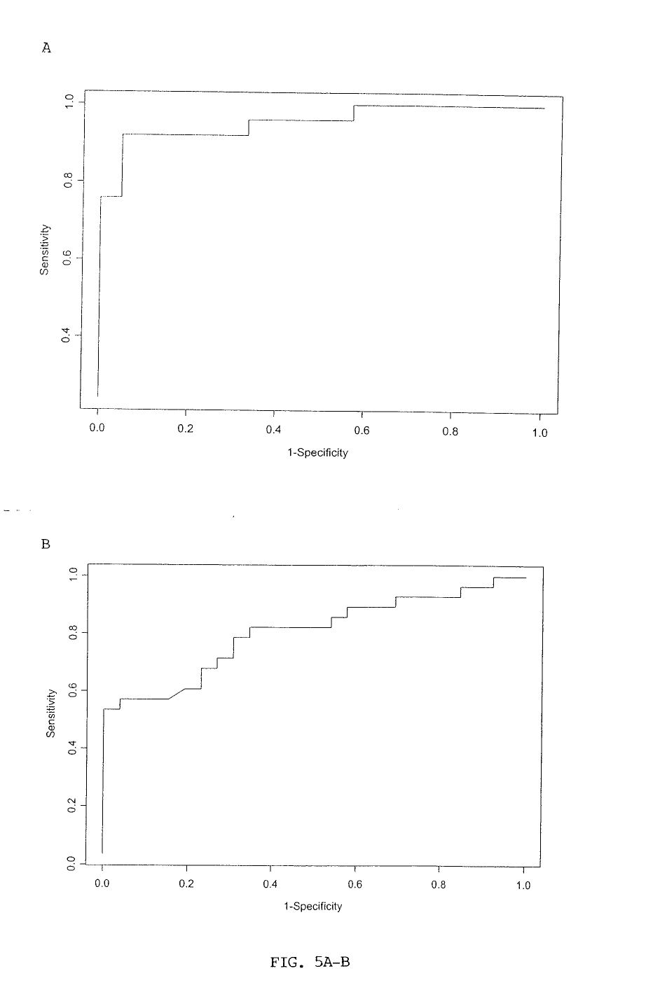

Summary statistics of patients' characteristics grouped by cancer status. Patients' characteristics such as age, smoking history, probe measurements, and image analysis parameters are summarized both in graphic and tabular formats. The BLiP-Plots (FIGS. 4A-V) display the distributions of patients' characteristics conditional on cancer status. The notation ‘cancer’ refers to malignant cases and ‘control’ refers to the benign cases.

Statistics such as the mean, standard deviation, median, and range for continuous variables are presented in Table 7(a). For categorical variables such as gender, counts and column percentages are shown in Table 7(b). In Table 7(a), For continuous variables, the Wilcoxon rank sum test was used to assess differences between cancer patients and non-cancer patients. There was no significant difference in age between these two groups of patients. However, non-cancer patients had statistically marginally significantly higher levels of pack year, years of smoking, and number of cigarettes per day than cancer patients with the p-values of 0.051, 0.051, and 0.065. Epithelial cep3 3p deletion, Epithelial cep3 polisomies, and Epithelial A levels were significantly lower in non-cancer patients than those in cancer patients with the p-value of 0.024, 0.017, and 0.0003, respectively. Furthermore, non-cancer patients had significantly higher levels of Neutrophils cep 10 10q deletion and Neutrophils cep10 polisomies (p-value: 0.009 and 0.010). Some image analysis parameters such as SDV I.O.D., SDV M.O.D., and SDV St. Dev. were significantly different between these two groups of patients (p-value: 0.002, 0.003, 0.020).

TABLE 7(a)
Patients' characteristics grouped by cancer status

| | Cancer | | | | | | | |
|---|---|---|---|---|---|---|---|---|
| Characteristics | Status | N | mean | std. | min | median | max | p-value |
| Age | | | | | | | | 0.31 |
| | Benign | 26 | 64.2 | 5.2 | 56 | 63.5 | 75 | |
| | Malignant | 27 | 66.6 | 8.7 | 47 | 65 | 80 | |
| Pack year | | | | | | | | 0.051 |
| | Benign | 25 | 53.0 | 25.4 | 0 | 45 | 110 | |
| | Malignant | 24 | 45.9 | 50.3 | 0 | 39 | 210 | |
| Year of smoking | | | | | | | | 0.051 |
| | Benign | 26 | 36.5 | 12.1 | 0 | 39 | 55 | |
| | Malignant | 22 | 28.3 | 15.9 | 0 | 34.5 | 51 | |
| Number of cigarettes | | | | | | | | 0.065 |
| per day | | | | | | | | |
| | Benign | 26 | 28.9 | 13.5 | 0 | 22.5 | 60 | |
| | Malignant | 22 | 23.5 | 24.7 | 0 | 20 | 120 | |
| Epithelial cep10 10q | | | | | | | | 0.59 |
| deletion | | | | | | | | |
| | Benign | 26 | 2.1 | 1.5 | 0 | 2 | 5 | |
| | Malignant | 28 | 2.4 | 1.6 | 0 | 2 | 6 | |
| Epithelial cep10 | | | | | | | | 0.14 |
| polisomies | | | | | | | | |
| | Benign | 26 | 1.3 | 1.3 | 0 | 1 | 4 | |
| | Malignant | 28 | 2.1 | 2.0 | 0 | 1 | 7 | |
| Epithelial cep3 3p | | | | | | | | 0.024 |
| deletion | | | | | | | | |
| | Benign | 26 | 2.7 | 1.5 | 0 | 3 | 6 | |
| | Malignant | 28 | 4.4 | 2.9 | 1 | 3 | 13 | |
| Epithelial cep3 | | | | | | | | 0.017 |
| polisomies | | | | | | | | |
| | Benign | 26 | 2.8 | 1.5 | 0 | 3 | 6 | |
| | Malignant | 28 | 4.5 | 2.4 | 1 | 4 | 10 | |
| Epithelial A | | | | | | | | 0.0003 |
| | Benign | 26 | 1.8 | 1.2 | 0 | 2 | 5 | |
| | Malignant | 28 | 4.2 | 2.4 | 1 | 4 | 8 | |
| Epithelial B | | | | | | | | 0.61 |
| | Benign | 26 | 0.6 | 0.6 | 0 | 1 | 2 | |
| | Malignant | 28 | 0.6 | 1.0 | 0 | 0 | 3 | |
| Epithelial C | | | | | | | | 0.095 |
| | Benign | 26 | 0.7 | 1.0 | 0 | 0 | 3 | |
| | Malignant | 28 | 1.4 | 1.4 | 0 | 1 | 5 | |
| Epithelial D | | | | | | | | 0.42 |
| | Benign | 26 | 0.3 | 0.6 | 0 | 0 | 2 | |
| | Malignant | 28 | 0.5 | 0.9 | 0 | 0 | 3 | |
| Neutrophils cep10 | | | | | | | | 0.009 |
| 10q deletion | | | | | | | | |
| | Benign | 21 | 2.1 | 1.2 | 0 | 2 | 4 | |
| | Malignant | 25 | 1.1 | 1.4 | 0 | 1 | 5 | |
| Neutrophils cep10 | | | | | | | | 0.010 |
| polisomies | | | | | | | | |
| | Benign | 21 | 2.1 | 1.3 | 0 | 2 | 5 | |
| | Malignant | 25 | 1.2 | 1.6 | 0 | 0 | 5 | |
| Neutrophils cep3 3p | | | | | | | | 0.55 |
| deletion | | | | | | | | |
| | Benign | 19 | 4.4 | 2.8 | 1 | 3 | 12 | |
| | Malignant | 23 | 4.0 | 3.2 | 0 | 3 | 15 | |
| Neutrophils cep3 | | | | | | | | 0.36 |
| polisomies | | | | | | | | |
| | Benign | 16 | 2.7 | 2.4 | 0 | 2.5 | 8 | |
| | Malignant | 22 | 2.1 | 2.8 | 0 | 1 | 9 | |
| Neutrophils A | | | | | | | | 0.10 |
| | Benign | 23 | 1.7 | 1.3 | 0 | 1 | 5 | |
| | Malignant | 26 | 3.0 | 2.5 | 0 | 3 | 7 | |
| Neutrophils B | | | | | | | | 0.95 |
| | Benign | 23 | 0.3 | 0.6 | 0 | 0 | 2 | |
| | Malignant | 26 | 0.2 | 0.5 | 0 | 0 | 2 | |
| Neutrophils C | | | | | | | | 0.57 |
| | Benign | 23 | 0.5 | 1.0 | 0 | 0 | 4 | |
| | Malignant | 26 | 0.4 | 0.8 | 0 | 0 | 3 | |
| Neutrophils D | | | | | | | | 0.14 |
| | Benign | 23 | 0.1 | 0.3 | 0 | 0 | 1 | |
| | Malignant | 26 | 0.4 | 0.6 | 0 | 0 | 2 | |
| SDV Area | | | | | | | | 0.12 |
| | Benign | 26 | 15.0 | 2.1 | 11.9 | 15.04 | 20.0 | |
| | Malignant | 28 | 16.5 | 3.8 | 10.3 | 16.3 | 28.7 | |
| SDV I.O.D. | | | | | | | | 0.002 |
| | Benign | 26 | 3504.2 | 1222.2 | 1610 | 3354.4 | 6830 | |
| | Malignant | 28 | 4948.6 | 1822.6 | 2740.1 | 4650.4 | 10068.9 | |
| SDV Image | | | | | | | | 0.14 |
| | Benign | 26 | 73.6 | 13.1 | 54.1 | 70 | 107.4 | |
| | Malignant | 28 | 71.1 | 18.8 | 46.5 | 64.1 | 133.5 | |
| SDV M.O.D. | | | | | | | | 0.003 |
| | Benign | 26 | 4.1 | 0.5 | 2.9 | 4.1 | 4.9 | |
| | Malignant | 28 | 4.7 | 0.9 | 3.3 | 4.7 | 7.4 | |
| SDV Skewness | | | | | | | | 0.67 |
| | Benign | 26 | 0.5 | 0.1 | 0.4 | 0.5 | 0.6 | |
| | Malignant | 28 | 0.5 | 0.1 | 0.4 | 0.5 | 0.6 | |
| SDV St. Dev. | | | | | | | | 0.020 |
| | Benign | 26 | 2.1 | 0.3 | 1.5 | 2.1 | 2.4 | |
| | Malignant | 28 | 2.4 | 0.5 | 1.6 | 2.3 | 4.1 | |

TABLE 7(b)
Patients' characteristics grouped by cancer status

| | | Cancer Status | | |
|---|---|---|---|---|
| | | Benign | Malignant | |
| | | (n = 26) | (n = 28) | p-value |
| Gender | | | | 0.52 |
| | Female | 11(42.3%) | 11(39.3%) | |
| | Male | 15(57.7%) | 17(60.7%) | |
| Smoking status | | | | 0.27 |
| | Current | 9(34.6%) | 5(18.5%) | |
| | Former | 14(53.9%) | 13(48.2%) | |
| | Recently | 1(3.9%) | 4(14.8%) | |
| | Quitter | | | |
| | Never | 2(7.7%) | 5(18.5%) | |
| Smoked ≧1000 | | | | 0.13 |
| in life | | | | |
| | Yes | 25(96.2%) | 18(81.8%) | |
| | No | 1(3.9%) | 4(18.2%) | |
| Cytology | | | | 0.089 |
| diagnosis | | | | |
| | Negative | 12(46.2%) | 7(25%) | |
| | or scanty | | | |
| | atypial | 3(11.5%) | 2(7.1%) | |
| | Squamous | 6(23.1%) | 16(57.1%) | |
| | metaplasia | | | |
| | Metaplasia/ | 5(19.2%) | 3(10.7%) | |
| | dysplasia + | | | |
| | severe squamous | | | |
| | dysplasia | | | |
| Epithelial B | | | | 0.41 |
| | 0 | 12(46.2%) | 17(60.7%) | |
| | >0 | 14(53.9%) | 11(39.3%) | |
| Epithelial C | | | | 0.089 |
| | 0 | 15(57.7%) | 10(35.7%) | |
| | >0 | 11(42.3%) | 18(64.3%) | |
| Epithelial D | | | | 0.53 |
| | 0 | 21(80.8%) | 20(71.4%) | |
| | >0 | 5(19.2%) | 8(28.6%) | |
| Neutrophils B | | | | 1.00 |
| | 0 | 19(82.6%) | 21(80.8%) | |
| | >0 | 4(17.4%) | 5(19.2%) | |
| Neutrophils C | | | | 0.75 |
| | 0 | 16(69.6%) | 20(76.9%) | |
| | >0 | 7(30.4%) | 6(23.1%) | |
| Neutrophils D | | | | 0.18 |
| | 0 | 20(87%) | 18(69.2%) | |
| | >0 | 3(13%) | 8(30.8%) | |

For the categorical variables, Fisher's exact test was used to assess the associations between patients' characteristics and cancer status. The Neutrophils B, C, D, and Epithelial B, C, D levels were 0 for most patients. Therefore, they were dichotomized into two groups (0 vs. >0) for the analysis performed in Section III.

Correlations between Epithelial probes and Neutrophils probes. Scatter plots of the relationship between Neutrophils and Epithelial probes are presented in Tables 8-11(b). The statistics on the upper right corner represent correlation coefficients and p-values from Spearman rank test, respectively.

TABLE 8
Associations between Probes and Types of Cancer

| Probe | Cancer Type | N | mean | std | min | median | max | p-value |
|---|---|---|---|---|---|---|---|---|
| Epithelial cep10 | | | | | | | | 0.26 |
| 10q deletion | | | | | | | | |
| | Non-adenocarcinoma | 13 | 2.77 | 1.59 | 1 | 2.0 | 6 | |
| | adenocarcinoma | 15 | 2.13 | 1.55 | 0 | 2.0 | 6 | |
| Epithelial cep10 | | | | | | | | 0.62 |
| polisomies | | | | | | | | |
| | Non-adenocarcinoma | 13 | 2.46 | 2.26 | 0 | 2.0 | 7 | |
| | adenocarcinoma | 15 | 1.87 | 1.77 | 0 | 1.0 | 6 | |
| Epithelial cep3 3p | | | | | | | | 0.69 |
| deletion | | | | | | | | |
| | Non-adenocarcinoma | 13 | 4.08 | 2.36 | 1 | 3.0 | 8 | |
| | adenocarcinoma | 15 | 4.73 | 3.26 | 1 | 3.0 | 13 | |
| Epithelial cep3 | | | | | | | | 0.62 |
| polisomies | | | | | | | | |
| | Non-adenocarcinoma | 13 | 4.62 | 2.14 | 2 | 5.0 | 8 | |
| | adenocarcinoma | 15 | 4.33 | 2.69 | 1 | 3.0 | 10 | |
| Epithelial A | | | | | | | | 0.39 |
| | Non-adenocarcinoma | 13 | 4.62 | 2.50 | 1 | 5.0 | 8 | |
| | adenocarcinoma | 15 | 3.80 | 2.31 | 1 | 4.0 | 8 | |
| Epithelial B | | | | | | | | 0.50 |
| | Non-adenocarcinoma | 13 | 0.77 | 1.01 | 0 | 0.0 | 3 | |
| | adenocarcinoma | 15 | 0.53 | 0.92 | 0 | 0.0 | 3 | |
| Epithelial C | | | | | | | | 0.87 |
| | Non-adenocarcinoma | 13 | 1.38 | 1.39 | 0 | 1.0 | 4 | |
| | adenocarcinoma | 15 | 1.33 | 1.50 | 0 | 1.0 | 5 | |
| Epithelial D | | | | | | | | 0.20 |
| | Non-adenocarcinoma | 13 | 0.31 | 0.85 | 0 | 0.0 | 3 | |
| | adenocarcinoma | 15 | 0.60 | 0.91 | 0 | 0.0 | 3 | |
| Neutrophils cep10 | | | | | | | | 0.11 |
| 10q deletion | | | | | | | | |
| | Non-adenocarcinoma | 11 | 1.55 | 1.63 | 0 | 1.0 | 5 | |
| | adenocarcinoma | 14 | 0.71 | 1.14 | 0 | 0.0 | 3 | |
| Neutrophils cep10 | | | | | | | | 0.15 |
| polisomies | | | | | | | | |
| | Non-adenocarcinoma | 11 | 1.73 | 1.90 | 0 | 1.0 | 5 | |
| | adenocarcinoma | 14 | 0.71 | 1.14 | 0 | 0.0 | 3 | |
| Neutrophils cep3 | | | | | | | | 0.61 |
| 3p deletion | | | | | | | | |
| | Non-adenocarcinoma | 9 | 4.67 | 4.09 | 2 | 3.0 | 15 | |
| | adenocarcinoma | 14 | 3.57 | 2.62 | 0 | 3.5 | 9 | |
| Neutrophils cep3 | | | | | | | | 0.52 |
| polisomies | | | | | | | | |
| | Non-adenocarcinoma | 8 | 2.38 | 2.33 | 0 | 2.5 | 6 | |
| | adenocarcinoma | 14 | 1.93 | 3.05 | 0 | 0.5 | 9 | |
| Neutrophis A | | | | | | | | 0.049 |
| | Non-adenocarcinoma | 11 | 4.09 | 2.51 | 0 | 3.0 | 7 | |
| | adenocarcinoma | 15 | 2.20 | 2.18 | 0 | 1.0 | 6 | |
| Neutrophis B | | | | | | | | 0.47 |
| | Non-adenocarcinoma | 11 | 0.27 | 0.47 | 0 | 0.0 | 1 | |
| | adenocarcinoma | 15 | 0.20 | 0.56 | 0 | 0.0 | 2 | |
| Neutrophis C | | | | | | | | 0.24 |
| | Non-adenocarcinoma | 11 | 0.45 | 0.69 | 0 | 0.0 | 2 | |
| | adenocarcinoma | 15 | 0.27 | 0.80 | 0 | 0.0 | 3 | |
| Neutrophis D | | | | | | | | 0.54 |
| | Non-adenocarcinoma | 11 | 0.45 | 0.69 | 0 | 0.0 | 2 | |
| | adenocarcinoma | 15 | 0.27 | 0.46 | 0 | 0.0 | 1 | |

TABLE 9
Univariate Logistic regression models in estimating cancer status

| | Parameter | Std | | Odds |
|---|---|---|---|---|
| Characteristics | estimate | error | p-value | ratio |
| Age | 0.05 | 0.04 | 0.23 | 1.05 |
| Pack year | −0.005 | 0.01 | 0.53 | 0.995 |
| Years of smoking | −0.04 | 0.02 | 0.060 | 0.96 |
| Number of cigarettes per day | −0.02 | 0.02 | 0.35 | 0.98 |
| Smoking status | | | | |
| Former vs. never | −1.46 | 1.18 | 0.22 | 0.23 |
| Current vs. never | −1.48 | 1.20 | 0.22 | 0.23 |
| Smoked ≧1000 in life | 1.71 | 1.16 | 0.14 | 5.56 |
| (yes. vs. no) | | | | |
| Cytology diagnosis (non- | −0.94 | 0.59 | 0.11 | 0.39 |
| ngative vs. negative) | | | | |
| Epithelial cep10 10q deletion | 0.16 | 0.19 | 0.39 | 1.17 |
| Epithelial cep 10 polisomies | 0.31 | 0.18 | 0.086 | 1.36 |
| Epithelial cep3 3p deletion | 0.40 | 0.17 | 0.017 | 1.49 |
| Epithelial cep3 polisomies | 0.45 | 0.17 | 0.009 | 1.57 |
| Epithelial sum* | 0.19 | 0.07 | 0.008 | 1.21 |
| Epithelial A | 0.68 | 0.20 | 0.001 | 1.97 |
| Epithelial B (>0 vs. 0) | −0.59 | 0.55 | 0.29 | 0.55 |
| Epithelial C (>0 vs. 0) | 0.90 | 0.56 | 0.11 | 2.45 |
| Epithelial D (>0 vs. 0) | 0.52 | 0.65 | 0.42 | 1.68 |
| Neutrophils cep10 10q deletion | −0.55 | 0.24 | 0.024 | 0.58 |
| Neutrophils cep10 polisomies | −0.48 | 0.22 | 0.034 | 0.62 |
| Neutrophils cep3 3p deletion | −0.04 | 0.10 | 0.69 | 0.96 |
| Neutrophils cep3 polisomies | −0.09 | 0.13 | 0.48 | 0.91 |
| Neutrophils sum* | −0.05 | 0.06 | 0.36 | 0.95 |
| Neutrophils A | 0.34 | 0.17 | 0.042 | 1.40 |
| Neutrophils B (>0 vs. 0) | 0.12 | 0.74 | 0.87 | 1.13 |
| Neutrophils C (>0 vs. 0) | −0.38 | 0.65 | 0.56 | 0.69 |
| Neutrophils D (>0 vs. 0) | 1.09 | 0.75 | 0.15 | 2.96 |
| SDV Area | 0.18 | 0.10 | 0.094 | 1.19 |
| SDV I.O.D. | 0.001 | 0.0002 | 0.005 | 1.001 |
| SDV Image | −0.01 | 0.02 | 0.58 | 0.99 |
| SDV M.O.D. | 1.45 | 0.54 | 0.007 | 4.26 |
| SDV Skewness | −3.57 | 4.60 | 0.44 | 0.03 |
| SDV St. Dev. | 2.36 | 0.94 | 0.012 | 10.58 |

*The sum of cep10 10q deletion, cep 10 polisomies, cep3 3p deletion, cep3 polisomies

TABLE 9(a)
Model A: Multivariate logistic regression
model in estimating cancer status

| | Parameter | Std | | Odds |
|---|---|---|---|---|
| Characteristics | estimate | error | p-value | ratio w/95% CI |
| Epithelial cep 10 | −2.10 | 1.13 | 0.063 | 0.12 (0.01, 1.12) |
| 10q deletion | | | | |
| Epithelial cep 10 | 2.43 | 1.02 | 0.017 | 11.4 (1.53, 84.2) |
| polisomies | | | | |
| Epithelial cep 3 | 1.80 | 0.82 | 0.029 | 6.08 (1.21, 30.6) |
| 3p deletion | | | | |
| Epithelial A | 1.30 | 0.61 | 0.032 | 3.67 (1.12, 12.1) |
| Neutrophils cep 10 | −1.81 | 0.68 | 0.007 | 0.16 (0.04, 0.61) |
| 10q deletion | | | | |

TABLE 9(b)
Model A: Comparison of cancer status and predicted cancer status

| | |
|---|---|
| Predicted | Observed cancer status |

| cancer status | Malignant (n = 25) | Benign (n = 21) |
|---|---|---|
| Malignant | 21 (84.0%) | 4 (19.1%) |
| Benign | 4 (16.0%) | 17 (81.0%) |

TABLE 10(a)
Model B: Multivariate logistic regression
model in estimating cancer status

| | Parameter | Std | | Odds |
|---|---|---|---|---|
| Characteristics | estimate | error | p-value | ratio w/95% CI |
| SDV I.O.D. | 0.001 | 0.0003 | 0.010 | 1.001 (1.00, 1.001) |
| SDV image | −0.03 | 0.02 | 0.12 | 0.97 (0.92, 1.01) |
| SDV M.O.D. | 1.02 | 0.59 | 0.083 | 2.77 (0.88, 8.71) |

TABLE 10(b)
Model B: Comparison of cancer status and predicted cancer status

| | |
|---|---|
| Predicted | Observed cancer status |

| cancer status | Malignant (n = 28) | Benign (n = 26) |
|---|---|---|
| Malignant | 19 (67.9%) | 6 (23.1%) |
| Benign | 9 (9.0%) | 20 (76.9%) |

TABLE 11(a)
Model C: Multivariate logistic regression
model in estimating cancer status

| | Parameter | Std | | Odds |
|---|---|---|---|---|
| Characteristics | estimate | error | p-value | ratio w/95% CI |
| Epithelial cep10 | 1.00 | 0.42 | 0.016 | 2.73 (1.20, 6.18) |
| polisomies | | | | |
| Epithelial cep 3 | 1.36 | 0.59 | 0.020 | 2.89 (1.23, 12.3) |
| 3p deletion | | | | |
| Neutrophils cep10 | −0.54 | 0.44 | 0.22 | 0.58 (0.24, 1.39) |
| 10q deletion | | | | |
| SDV I.O.D. | 0.001 | 0.001 | 0.013 | 1.001 (1.00, 1.002) |
| SDV image | −0.14 | 0.06 | 0.020 | 0.87 (0.77, 0.98) |

TABLE 11(b)
Model C: Comparison of cancer status and predicted cancer status

| | |
|---|---|
| Predicted | Observed cancer status |

| cancer status | Malignant (n = 25) | Benign (n = 21) |
|---|---|---|
| Malignant | 23 (92.0%) | 4 (19.0%) |
| Benign | 2 (8.0%) | 17 (81.0%) |

Epithelial cep10 10q and Neutrophils cep 10 10q were marginally significantly correlated with each other with the p-value of 0.056. The Epithelial cep3 polisomes was significantly correlated with the Neutrophils cep3 polisomies with the p-value of 0.001. The correlation between Epithelial A and Neutrophils A was marginally significant (p-value: 0.076).

The distributions of probe measurements between different types of cancers were compared using the Wilcoxon rank sum test. All cancer patients were dichotomized into two groups: adenocarcinoma patients and patients with non-adenocarcinoma forms of lung cancer. With one exception (Neutrophils A), the probe measurements were well balanced between different types of cancer. The Neutrophils A levels were significantly higher in the non-adenocarcinoma patients than those in adenocarcinoma patients.

Prediction of patients' cancer status. Univariate logistic regression models were performed to assess how patients' characteristics were associated with cancer status. Patients' years of smoking were marginally significantly associated with cancer status. The increase in Epithelial cep 3 3p deletion, Epithelial cep 3 polisomies, Epithelial sum, and Epithelial A levels statistically significantly increased the odds of cancer. The increase of Neutrophils cep10 10q deletion and Neutrophils cep 10 polisomies significantly decreased the odds of cancer with the p-value of 0.024 and 0.034. The increase of Neutrophils A level was also significantly associated with the increasing risk of cancer (p-value: 0.042). The image analysis parameters such as SDV I.O.D., SDV M.O.D., and SDV St. Dev. were significantly positively associated with cancer status.

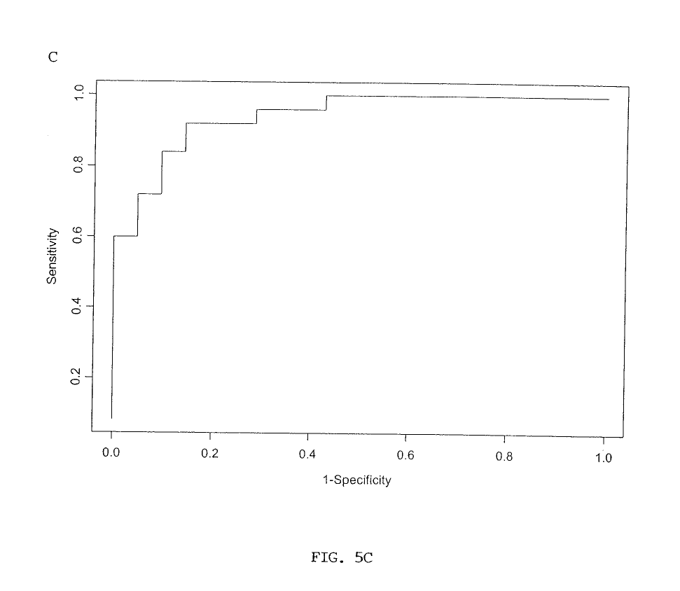

After model selections, several multivariate logistic regression models were identified to predict cancer status. The predicted cancer status using leave-one-out algorithm based on each multivariate logistic regression models was compared to the true cancer status. The receiver operating characteristic (ROC) curve for each model was presented in the FIGS. 5A-C. Receiver operating characteristic (ROC) curve is produced for this prediction model. It is a plot of the true positive rate against the false positive rate for the different possible cut points of a diagnostic test. An ideal prediction model will have 100% sensitivity (true positive) and 100% specificity (true negative). ROC captures the information how good a prediction model is.

Model A predicts cancer status based on the probe measurements alone. Model B is the prediction model using the image analysis parameters only. Model C combines probe measurements and image analysis parameters together. Using leave-one-out algorithm, Models C is the best model in terms of predictions among three models. The sensitivity and specificity are 92% and 81%.

### Example 4

As discussed above, selected probes targeted at chromosome regions (3p21.3, 10q23 (surfactant protein A gene) and 5p13.2 (gene encoding H-TERT) have permitted the detection of locus-specific changes in tumors, preneoplastic cells, circulating tumor cells. Table 12, below, presents updated information on FISH tests using the above-mentioned probes together with centromeric probes for chromosomes 3, 10 and 5 respectively. These latter probe sets have been used on five different sites throughout the upper and lower respiratory tract, including the tumor (TTP), the adjacent bronchus (TAB), normal lung distal to the tumor (NTP) on both the sides of the tumor (TBB/ipsilateral and contralateral/NBB) testing the bronchial brush specimens.

The inventors are continuing to process blood and bronchial brushing samples, and tumor tissue and to apply the assays. In fact, they have had performed FISH for deletions and molecular aberrations of 3p22.1 and 10q22-23 on 119 bronchial brushes including both tumor and normal sides as well as close to 100 tumor imprints. Within the tumor cells, the mean percentage of 3p deletions was 16%+/−10, and for 10q deletions was 11%+/−7.

TABLE 12
Summary of Variables in Data Sets Target
drc, Spore data, Fish, and Comet Target

| Data Set | Variable | N | Mean | SD | Minimum | Maximum |
|---|---|---|---|---|---|---|
| Spore_data | Control cep3/3p (% Del) | 120 | 2.53 | 1.06 | 0 | 4 |
| | cep3/3p-NBB (% Del) | 119 | 2.12 | 1.79 | 0 | 8 |
| | cep3/3p-TBB (% Del) | 120 | 5.48 | 4.01 | 0 | 30 |
| | cep3/3p-NTP (% Del) | 96 | 3.16 | 2.88 | 0 | 14 |
| | cep3/3p-TAB (% Del) | 71 | 4.2 | 2.95 | 0 | 16 |
| | cep3/3p-TTP (% Del) | 97 | 15.96 | 9.58 | 3 | 53 |
| | control cep10/10q (% Del) | 120 | 2.67 | 0.98 | 0 | 4 |
| | cep10/10q-NBB (% Del) | 119 | 1.21 | 1.34 | 0 | 6 |
| | cep10/10q-TBB (% Del) | 120 | 3.85 | 2.04 | 0 | 11 |
| | cep10/10q-NTP (% Del) | 94 | 2.23 | 1.98 | 0 | 9 |
| | cep10/10q-TAB (% Del) | 69 | 4.18 | 2.51 | 1 | 14 |
| | cep10/10q-TTP (% Del) | 97 | 11.03 | 6.61 | 2 | 29 |

Abbreviations: BBT, bronchial brushings on tumor side; BBN, bronchial brushings on normal side; TT, tumor tissue; NT, adjacent normal tissue.

The field cancerization effect documented previously has persisted in the larger sample number, with higher percentages of deletions for 3p and 10 q being noted in the normal cells on the bronchial brush on the side of the tumor as compared to the normal side. These results were subsequently validated in a random subset of repeat FISH counts done by an independent cytogenetic lab on 40 cases or 80 slides. There was a good correlation between the results of both labs by the Pearson correlation coefficient (0.01).

### Example 5

### Telomere Length

The inventors have also assayed the gene for 5p13 (H-Tert) in 133 and 135 NBB, TBB and TTP samples. By scoring an internal control probe for centromeric 5, a ratio for 5p is established on a per case basis using the formula: total 5p copies in 100 cells divided by total centromeric 5 in 100 cells. A 5p ratio greater than 1.0 is indicative of over-expression of HTert. Ratios were calculated for NBB, TBB, TPP. Telomere lengths of cells (TL), using a quantitative image analysis assay performed interactively via a software program, were calculated in these same sites.

The Htert or 5p ratio was highest in the TTP, followed by the TBB and lowest on the NBB side. The 5p ratio on the NBB side correlated inversely with the 10q deletions on both the NBB and TBB sides (p=0.027, p=0.034) and inversely and significantly with the telomerase score. TL were shortest in the TTP (3.89), and longest in the TBB (4.31) with the NBB having TL intermediate between these sites (4.04). The TTP 5p ratio was significantly inversely related to the TL in the tumor cells (0.011), thus the higher the HTert ratio the shorter the TL and vice versa. The deletion of 10q in the TTP trended to be inversely correlated with the 5p ratio (p=0.14).

Using an immunocytochemical assay, the inventors also assayed the level of telomerase in a microarray slide set of the same tumor touch imprints. The mean telomerase score was 164 however there was a large SD of 72. The association between 5p ratio at all sites, histologic diagnosis, smoking, survival, relapse and pathological staging of lung showed no significant correlation, except for the 5p ratio of TTP that trended towards higher pathological stage of lung cancer. (p=0.07).

Finally, although a small subset, TL length in tumor cells was significantly inversely correlated with 10q deletions in tumor cells (p=0.001) whereas TL in NBB and TBB cells correlated positively with 10q deletions in TBB (p=0.029, p=0.059). TL in the tumor cells correlated positively with 3p deletions in tumor cells (p=0.004). Finally there was a significant association between TL in TTP and histological diagnosis with adenocarcinoma cells having significantly shorter TL than squamous carcinoma (p=0.04).

There was a significant difference between stage of lung cancer and percentage of deletions for surfactant protein A gene in the malignant cells of the touch imprint taken from the non-small cell carcinomas that were resected. Patients with high stage disease comprising 29 patients (stages III and IV) had a significantly higher level of deletions than 66 patients with stages I and II disease (p=0.007) (Table 13). Further more higher deletions of 10q22-23 (surfactant protein A gene) were associated with lymph node metastases.

TABLE 13
Association Between Variables cep10/10q-TTP (% Del) and Recurrence,
Sex, Lymph Nodes or Pathological Stage in data set Spore Data

| | | | | | | | | P-value |
|---|---|---|---|---|---|---|---|---|
| | | | | | | | | (Wilcoxon |
| Group | Type | N | Mean | SD | Median | Min | Max | Rank-sum Test) |
| Recurrence | 0 | 80 | 11.35 | 6.89 | 9 | 2 | 29 | 0.64 |
| | 1 | 17 | 9.53 | 4.98 | 9 | 4 | 25 | |
| Sex | F | 53 | 11.45 | 7.30 | 9 | 2 | 29 | |
| | M | 43 | 10.3 | 5.59 | 9 | 3 | 29 | 0.89 |
| Lymph_Nodes | 1 | 75 | 10.08 | 5.97 | 8 | 2 | 29 | |
| | 2 | 17 | 13.59 | 7.20 | 10 | 4 | 25 | 0.03 |
| | 3 | 5 | 16.60 | 9.96 | 18 | 4 | 27 | 0.16 |
| Pathological | 0, I, II | 66 | 10.00 | 5.93 | 8 | 2 | 27 | |
| Stage | III, IV | 29 | 13.86 | 7.35 | 10 | 4 | 29 | 0.007 |

Thus, in summary, the data indicate that the presence of 10q23 deletion in tumor cells associates significantly with longer telomeres, higher pathological stage and over-expression of H-TERT. Elongation and maintenance of telomere length is a characteristic of malignant cells as normal somatic cells usually undergo apoptosis and senescence. Telomerase is also not usually expressed in normal somatic cells and is associated with regulation of the promoter region of the H-TERT gene by C-myc and Sp1 Overexpression of c-Myc has been frequently found in non-small cell lung cancer and is associated with high stage lung cancer. Based on these findings, it would appear that the gene for 10q23, or its product surfactant protein A, may act to repress the transcription of telomerase. Deletion of 10q, which the inventors have shown to be associated with smoking, may be one of the causes of activation of telomerase and may be an important pathway in tumorigenesis of lung cancer.

### Example 6

### Curcumin Chemoprevention Study

Background. Patients with chronic obstructive airways disease (COPD) and heavy smoking history are at higher risk to develop lung cancer than the general smoking public without COPD. A chemopreventive agent, curcumin, has been shown to be anti-tumorogenic by decreasing inflammation, down regulating cellular proliferation and angiogenesis and up-regulating apoptosis via suppression of an oncogene known as NF-kappa B. In addition, Bioperine, a 95% pure piperine obtained from the fruits of black pepper through a proprietary extraction process (Sabinsa Corp., Piscataway, N.J.), was used in this study.

The purpose of the study was to see if the patients on curcumin would have improved lung function over time due to down regulation of the inflammatory response. FISH analysis was performed to evaluate if the curcumin, compared to the placebo, could modulate the FISH abnormalities over time. The inventors evaluated induced sputum samples at baseline and at subsequent intervals over a period of three months during which time 16 patients received curcumin and 4 patients received placebo.

Each specimen was evaluated for adequacy (based on the presence of >5 macrophages), and classified as negative or squamous metaplasia, atypical squamous metaplasia, atypia, or dysplasia graded as mild, moderate and severe. In addition a cell count encompassing the percentages of neutrophils, eosinophils, lymphocytes, macrophages, bronchial epithelial and squamous cells was performed.

Atypical sputum cytology, at the moderate or severe dysplasia stage, has been shown to be a predictor of development of non-small cell carcinoma of lung over time. In addition, the inventors' probes have been shown to be abnormally expressed in patients who have carcinoma of lung compared to controls without smoking and may be more sensitive than cytology to predict presence of cancer.

The inventors used DNA probes to 3p22.1, 3, 10 and 10q22-23 by fluorescence in situ hybridization (FISH) as surrogate biomarkers to assess response to curcumin (a natural product that acts by suppressing NF-κB) versus placebo in a cohort of patients from the VA hospital, who were heavy smokers and had chronic obstructive airways disease. Patients were followed over a three-month period and were randomized to the active group (with drug) and an inactive group (placebo). Lung function tests, cytology of sputum and quantiation of the inflammatory response were evaluated at baseline and over a three month period. There were 16 active patients and 4 placebo patients. FISH for quantitating the probes was performed both manually (M) and on an automated machine (B) without knowledge of patient's drug status. Both epithelial cells and neutrophils from sputum samples were analyzed for percentage deletions and aneusomies at baseline and after a three month period.

Results. Results of the study showed that there was a trend for FISH markers for aneuploidy of chromosome 10 (monosomy and polysomy) and deletions of 10q2-23 in both neutrophils and epithelial cells to be modulated downward over time in patients with the curcumin compared to the placebo. The effect was most marked for aneusomies of chromosome 10 in both epithelial cells and in neutrophils (p=0.8). Similarly there appeared to be a decrease in 3p deletions and aneusomies of centromeric 3 over time (Table 14, see below). In addition there was significant improvement in lung function over time in patients versus placebo. There was however no significant difference in diagnosis and degree of inflammation over time.

The inventors conclude that DNA probes against 3, 3p, 10 and 10q that detect genomic instability or chromosomal aberrations by FISH in sputa samples appear to be sensitive for detecting baseline genetic instability and may be modulated by effects of curcumin over time. Curcumin is a paradigm for other natural products with chemopreventive effects, and these FISH probes may be used as surrogate markers to monitor chemopreventive agents over time.

TABLE 14
FISH Analysis on 3p and 10q, Modulation Over Time

| | | | Mean ± std, Median | p- |
|---|---|---|---|---|
| Variable* | Treatment | N | (Minimum, Maximum) | value** |
| DEL_3P_EPI_M_diff | Active | 17 | −1.12 ± 2.91, −1 (−5, 4) | .27 |
| | Placebo | 4 | −3 ± 2.16, −2.5 (−6, −1) | |
| DEL_3P_EPI_B_diff | Active | 16 | −1.48 ± 1.5, −1.84 (−3.74, 1.38) | .43 |
| | Placebo | 4 | −0.66 ± 1.75, −0.31 (−3.07, 1.05) | |
| ANEU_3P_EPI_M_diff | Active | 17 | −1.06 ± 3.34, 0 (−7, 4) | .33 |
| | Placebo | 4 | −3 ± 4.08, −1.5 (−9, 0) | |
| ANEU_3P_EPI_B_diff | Active | 16 | −0.47 ± 2.16, 0.21 (−5.12, 2) | .27 |
| | Placebo | 4 | −0.88 ± 0.66, −0.95 (−1.6, 0) | |
| DEL_3P_NEUTRO_M_diff | Active | 17 | −0.06 ± 3.34, 0 (−10, 5) | .72 |
| | Placebo | 4 | −0.75 ± 2.99, 0 (−5, 2) | |
| DEL_3P_NEUTRO_B_diff | Active | 16 | −0.22 ± 1.86, 0 (−6.66, 2.38) | .48 |
| | Placebo | 4 | 0.33 ± 0.65, 0 (0, 1.3) | |
| ANEU_3P_NEUTRO_M_diff | Active | 17 | −0.15 ± 3.41, 0 (−10, 7) | 1.00 |
| | Placebo | 4 | −0.75 ± 2.99, 0 (−5, 2) | |
| ANEU_3P_NEUTRO_B_diff | Active | 16 | −0.51 ± 1.16, 0 (−4.16, 0.54) | .15 |
| | Placebo | 4 | 0.35 ± 0.7, 0 (0, 1.4) | |
| DEL_3P_EPI_Neutro_M_diff | Active | 17 | −1.18 ± 5.33, 0 (−15, 7) | .22 |
| | Placebo | 4 | −3.75 ± 4.86, −1.5 (−11, −1) | |
| DEL_3P_EPI_Neutro_B_diff | Active | 16 | −1.69 ± 2.5, −1.76 (−8.9, 2.38) | .22 |
| | Placebo | 4 | −0.33 ± 1.18, −0.31 (−1.77, 1.05) | |
| ANEU_3P_EPI_Neutro_M_diff | Active | 17 | −1.21 ± 5.27, −1 (−15, 6) | .69 |
| | Placebo | 4 | −3.75 ± 7.04, −1.5 (−14, 2) | |
| ANEU_3P_EPI_Neutro_B_diff | Active | 16 | −0.98 ± 3.05, 0.02 (−9.02, 2) | .61 |
| | Placebo | 4 | −0.53 ± 1.32, −0.95 (−1.6, 1.4) | |
| DEL_10Q_EPI_M_diff | Active | 16 | −0.69 ± 2.39, −0.5 (−7, 4) | .35 |
| | Placebo | 4 | −1.75 ± 2.06, −1.5 (−4, 0) | |
| DEL_10Q_EPI_B_diff | Active | 15 | −0.1 ± 2.55, 0 (−6.97, 3.73) | .35 |
| | Placebo | 4 | −2.54 ± 4.4, −1.8 (−8.24, 1.69) | |
| ANEU_10Q_EPI_M_diff | Active | 16 | −1.13 ± 2.83, −1 (−5, 4) | .51 |
| | Placebo | 4 | −0.25 ± 0.96, −0.5 (−1, 1) | |
| ANEU_10Q_EPI_B_diff | Active | 15 | −1.65 ± 3.89, −1.15 (−13.97, 2.7) | .16 |
| | Placebo | 4 | 0.25 ± 0.8, 0 (−0.42, 1.4) | |
| DEL_10Q_NEUTRO_M_diff | Active | 16 | −0.41 ± 3.43, 0 (−11, 7) | 1.00 |
| | Placebo | 4 | −0.25 ± 1.26, 0 (−2, 1) | |
| DEL_10Q_NEUTRO_B_diff | Active | 15 | −0.1 ± 0.85, 0 (−2.9, 1.35) | .94 |
| | Placebo | 4 | 0.47 ± 1.3, 0 (−0.51, 2.38) | |
| ANEU_10Q_NEUTRO_M_diff | Active | 16 | −0.34 ± 3.52, 0 (−11, 7) | .36 |
| | Placebo | 4 | −0.5 ± 1, 0 (−2, 0) | |
| ANEU_10Q_NEUTRO_B_diff | Active | 15 | −0.71 ± 2.56, 0 (−9.8, 0.8) | .25 |
| | Placebo | 4 | 0.4 ± 0.79, 0 (0, 1.58) | |
| DEL_10Q_EPI_Neutro_M_diff | Active | 16 | −1.09 ± 5.53, −0.75 (−18, 11) | .48 |
| | Placebo | 4 | −2 ± 2.45, −1.5 (−5, 0) | |
| DEL_10Q_EPI_Neutro_B_diff | Active | 15 | −0.21 ± 3.21, 0 (−9.87, 3.73) | .46 |
| | Placebo | 4 | −2.07 ± 4.61, −0.61 (−8.75, 1.69) | |
| ANEU_10Q_EPI_Neutro_M_diff | Active | 16 | −1.47 ± 5.7, −1 (−16, 11) | .85 |
| | Placebo | 4 | −0.75 ± 0.5, −1 (−1, 0) | |
| ANEU_10Q_EPI_Neutro_B_diff | Active | 15 | −2.37 ± 4.58, −0.5 (−13.97, 2.7) | .08 |
| | Placebo | 4 | 0.64 ± 0.75, 0.58 (0, 1.4) | |
| ABNORMAL_EPI_M_diff | Active | 16 | −4.5 ± 8.43, −4.5 (−22, 12) | .52 |
| | Placebo | 4 | −8 ± 8.29, −5 (−20, −2) | |
| ABNORMAL_EPI_B_diff | Active | 15 | −3.32 ± 6.78, −1.95 (−21.09, 6.43) | .73 |
| | Placebo | 4 | −3.83 ± 4.28, −2.65 (−9.8, −0.2) | |

*_M—Manual counts, _B—Bioview, _EPI_Neutro—sum of counts in epithelial and neutrophil, _diff—3-Month value—Baseline value.
ABNORMAL_EPI_M = EL_3P_EPI_M + ANEU_3P_EPI_M + DEL_10Q_EPI_M + ANEU_10Q_EPI_M
ABNORMAL_EPI_B = DEL_3P_EPI_B + ANEU_3P_EPI_B + DEL_10Q_EPI_B + ANEU_10Q_EPI_B
**Wilcoxon rank-sum test comparing values between two treatment groups.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the methods described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference:
U.S. Pat. No. 4,683,195
U.S. Pat. No. 4,683,202
U.S. Pat. No. 4,800,159
U.S. Pat. No. 4,883,750
U.S. Pat. No. 5,279,721
U.S. Pat. No. 5,610,287
U.S. Pat. No. 6,797,471
Alberola et al., Proc. Annu. Mt. Am. Soc. Clin. Oncol., 14: A1094, 1995.
Auerbach et al., N. Engl J Med., 265: 253-267, 1961.
Ayabe et al., Lung Cancer, 11(3-4): 201-208, 1994.
Bakhshi et al., Cell, 41(3):899-906, 1985.
Barinaga, Science, 271: 1233, 1996.
Bartlett et al., J. Pathology, 195:422-428, 2001.
Betz et al., Cancer Research, 55:4283-4286, 1995.
Broers et al., Lab. Invest., 66:337-346, 1992.
Brugal et al., Method. Achiev. Exp. Pathol, (Karger, Base1) 11: 1-33, 1984.
Carcy et al., JNCI, 65: 1225-1230, 1980.
Carriaga et al., Cancer, 75: 406-421, 1995.
Cheon et al., Yonsei Med. J, 34(4): 365-370, 1993.
Cleary and Sklar, Proc. Natl. Acad. Sci. USA, 82(21):7439-7443, 1985.
Cleary et al., J. Exp. Med., 164(1):315-320, 1986.
Cox et al., Thorax, 56:561-566, 2001.
Dalquen et al., Virchows Archiv., 431(3): 13-179, 1997.
Dong et al., Science, 268: 884-886, 1995.
Ekins and Chu, Trends in Biotechnology, 17: 217-218, 1999.
European Appln. 320 308
European Appln. 329 822
Feder et al., Cancer Genet. Cytogenet., 102: 25-31, 1998.
Feinstein et al., Am. Rev. Repir. Dis., 101: 671-684, 1970.
Fodor et al., Science, 251:767-773, 1991.
Fontanini et al., Cancer, 70(6): 1520-7, 1992.
Frohman, M. A., In: PCR PROTOCOLS: A GUIDE TO METHODS AND APPLICATIONS, Academic Press, N.Y., 1990
Fujita et al., Lung Cancer, 39:31-36, 2003.
GB Appln. 2 202 328
Hacia et al., Nature Genetics, 14:441-447, 1996.
Harbeck et al., Intl. J. Oncology, 14:663-671, 1999.
Hirano et al., American J. Path., 144(2): 296-302, 1994.
Hirsh, Manksgaard, 1-78, 1983.
Holmstrom et al., Anal Biochem. 209:278-283, 1993.
Ichinose et al., J. Surgical Oncology, 46(1): 15-20, 1991.
Ihde, Curr. Prob. Cancer, 15: 65, 1991.
Innis, et al., In: PCR Protocols. A guide to Methods and Application, Academic Press, Inc. San Diego, 1990.
Jiang and Katz, Diag. Molec. Pathology, 11:47-57, 2002.
Kerr et al., Br. J. Cancer, 26(4):239-257, 1972.
Kim et al., Korean J. Intern Med., 11(2): 101-7, 1996.
Kuroki et al., Amer. Rev. Respir. Disease, 147: 723-729, 1993.
Kwoh et al., Proc. Nat. Acad. Sci. USA, 86: 1173, 1989.
Licciardello et al., Int. J. Radiat. Oncol Bio. Phys., 17: 467-476, 1989.
Liewald et al., Chirurg, 63(3): 205-10, 1992.
Lifton, Science, 272: 676, 1996.
Linnoila et al., J. Natl. Cancer Instit. Monographs, 61-66, 1992.
Lukas et al., Nature 375(6531):503-6, 1995.
Macchiarini et al., Proc Annu Mt. Am. Soc. Clin. Oncol. 11: A995, 1992.
Miki et al., Science 266: 66-71, 1994.
Mitsudomi et al., Clin. Cancer Res., 2(7): 1185-9, 1996.
Miyamoto et al., Cancer Research, 51(23-pt1) 6346-50, 1991.
Morahan et al., Science 272: 1811, 1996.
Mrkve et al., Anticancer Research, 13(3): 571-8, 1993.
Muguerza et al., World J. Surg. 21(3): 323-329, 1997.
Naruke et al., J. Thorac. Cardiovas Surg, 96: 400, 1988.
Newton, C. R, et al. Nucl. Acids Res. 21:1155-1162 (1993).
Ohara et al., Proc. Nat'l Acad. Sci. USA, 86:5673-5677, 1989.
O'Reilly et al., Biochim. Biophys. Acta, 970:194-204, 1988.
Pantel et al., Proc. Annu Mt. Am. Soc. Clin. Oncol, 12: A941, 1993.
Papadimitrakopoulou et al., Cancer and Metastasis Reviews, 15: 53-76, 1996.
Pauletti et al., J. Clinical Oncology, 18:3651-3664, 2000.
PCT Appln. PCT/US87/00880
PCT Appln. PCT/US89/01025
PCT Appln. WO 88/10315
PCT Appln. WO 89/06700
PCT Appln. WO 90/07641
Pease et al., Proc. Natl. Acad. Sci. USA, 91:5022-5026, 1994.
Pence et al., Archives of Surgery, 128(12): 1382-1390, 1993.
Physician's Desk Reference, 56^{th} Ed. Medical Economics Co., Inc., NJ, 1862-1866, 2002.
Pignon et al., Hum. Mutat., 3: 126-132, 1994.
Rasmussen, et al., Anal Biochem, 198:138-142, 1991.
Remington's Pharmaceutical Sciences, 15^{th} ed., pages 1035-1038 and 1570-1580, Mack Publishing Company, Easton, Pa., 1980.
Rice et al., J. Thoracic Cardio. Surgery, 106(2): 201-217, 1993.
Running. J. A. et al., BioTechniques 8:276-277, 1990.
Sahin et al., Cancer, 65(3): 530-7, 1990.
Saiki et al., Science, 239: 487-491, 1988.
Sambrook et al., (ed.), Molecular Cloning, Cold Spring Harbor Laboratory Press, Cold Spring Harbor, N.Y., 1989.
Shijubo et al., Cancer, 69:2905-2909, 1992.
Shijubo et al., European Respir. J., 8:403-406, 1995.
Shiseki et al., Genes Chromosomes Cancer, 17(2): 71-7, 1996.
Shoemaker et al., Nature Genetics 14:450-456, 1996.
Shriver et al., Mutat. Res. 406(1): 9-23, 1998.
Sidransky et al., Science, 252: 706-709, 1991.
Slamon et al., Science, 244: 707-712, 1989.
Slaughter et al., Cancer, 6:963-968, 1953.
Smith and Rutledge, Natl. Cancer Inst. Monogr., 42:141-143, 1975.
Taparowsky et al., Nature, 300: 762-764, 1982.
Thiberville et al., Cancer Research, 55: 5133-5139, 1995a.
Thiberville et al., Int. J. Cancer, 64: 371, 1995b.
Travis et al., Cancer, 75: 191-202, 1995.
Tsujimoto and Croce, Proc. Natl. Acad. Sci. USA, 83(14):5214-5218, 1986.
Tsujimoto et al., Nature, 315:340-343, 1985.
Tsujimoto et al., Science, 228(4706):1440-1443, 1985.
Tsutsumida et al., Histopathology, 44:147-155, 2004.
Valdivieso et al., Proc. Annu. Mt. Am. Soc. Clin. Oncol, 13: A1121, 1994.
Vallmer et al., Hum. Pathol., 16: 247-252, 1985.
Volm et al., Versicherungsmedizin, 41(1): 2-5, 1989.
Voravud et al., Cancer Research, 53: 2874-2883, 1993.
Walker et al., Nucleic Acids Res. 20(7):1691-1696, 1992.
Winston et al., Amer. J. Clinical Pathology, 121:S33-49, 2004.
Wistuba et al., Cancer Res., 60(7): 1949-60, 2000.
Wong et al., Lung Cancer, 29:11-22, 2000.
Wu et al., Cancer Res., 58(8): 1605-8, 1998.
Wu et al., Proc. Natl. Acad. Sci. USA, 86(8):2757-2760, 1989.
Yamaoka et al., J. Japan Surgical Soc., 91(10): 1608-16, 1990.
Yanagisawa et al., Cancer Research, 56: 5579-5582, 1996.
Young et al., N Engl J Med. 7:299(23):1261-1266, 1978.
Zamecnik and Kodet, Virchows Archiv., 440:353-361, 2002.
Zou et al., Clinical Cancer Research 4: 1345-1355, 1998.

What is claimed is:

1. A method for identifying a subject at risk for the development of cancer comprising:
(a) providing probes for cen3 and cen17;
(b) contacting said probes with a nucleic acid test sample from said subject; and
(c) analyzing the hybridization pattern of said probes to said nucleic acid test sample,
whereby aberrations in the hybridization of said probes to said nucleic acid test sample, as compared to a normal nucleic acid sample, indicates that said subject is at risk for the development of cancer.

2. The method of claim 1, wherein said test sample comprises a surgical or biopsy specimen, a paraffin embedded tissue, a frozen tissue imprint, sputum, a lavage, peripheral blood, a bladder washing or barbotage, renal pelvic brushes, conduit urine, voided urine, esophageal brush, a fine needle aspirate, a buccal smear, spinal fluid, or serous cavity effusions such as pleural fluid or ascites.

3. The method of claim 1, wherein said cancer is lung cancer.

4. The method of claim 3, wherein said cancer is non-small cell lung cancer or small cell lung cancer.

5. The method of claim 1, wherein said cancer is an upper airway primary or secondary cancer.

6. The method of claim 1, wherein said cancer is bladder cancer.

7. The method of claim 1, wherein said cancer is cancer of the head or neck.

8. The method of claim 1, wherein said cancer is urothelial.

9. The method of claim 1, wherein said cancer is cancer of the kidneys.

10. The method of claim 1, wherein said cancer is cancer of the pancreas.

11. The method of claim 1, wherein said cancer is cancer of the mouth, throat, pharynx, larynx, or esophagus.

12. The method of claim 1, wherein said subject is a smoker.

13. The method of claim 1, wherein said subject is a former smoker.

14. The method of claim 1, wherein said subject is a non-smoker.

15. The method of claim 1, wherein said subject has not previously been diagnosed with cancer.

16. The method of claim 1, wherein said probe is labeled with a fluorophore.

17. The method of claim 1, wherein said probe size is between 100,000 and 300,000 base pairs.

18. The method in claim 1, further comprising a spiral CT-scan or an endoscopic evaluation of the bronchial tree of said subject.

19. The method of claim 1, further comprising administering to said subject chemopreventive drugs, nutritional supplements, cytokine, radiation, chemotherapeutic drugs or biological modifying response drugs, gene therapy, siRNA therapy or stem cells.

20. The method of claim 1, further comprising making a decision on whether said subject is in need of an intensive follow-up protocol.

21. The method of claim 1, further comprising making a determination of whether said subject is responding to a therapy.

22. The method of claim 1, wherein analyzing comprises FISH.

23. The method of claim 1, wherein said nucleic acid test sample is subject to separation on the basis of cell type prior to step (b).

24. The method of claim 1, wherein said abberations in hybridization are caused by deletions, amplifications or polysomies in regions corresponding to said probes.

25. The method of claim 23, wherein said cell type comprises cancer cells, lymphocytes, monocytes, histiocytes, neutrophils and/or epithelial cells.

26. The method of claim 25, wherein said neutrophils are granulocytes.

27. The method of claim 1, further comprising taking a patient history.

28. The method of claim 27, wherein said patient history may comprise smoking history, presence or absence of morphologic changes in sputum morphology (squamous metaplasia, dysplasia, etc.) and a genetic instability score.

29. The method of claim 1, further comprising:
(i) providing a probe for cen1;
(ii) contacting said cen 1 probe with a nucleic acid test sample from said subject; and
(iii) analyzing the hybridization pattern of said cen1 probe to said nucleic acid test sample.

30. The method of claim 1, further comprising analyzing the hybridization pattern of one or more probes for 3p22.1, 1q21, 9p21.3, 10q22, cen7 or cen10.

29. A method for identifying a subject at risk for the recurrence of cancer comprising:
(a) providing probes for 3p22.1, 1q21, 9p21.3, 10q22, cen17, and one or more of cen1, cen3, cen9, and cen10;
(b) contacting said probes with a nucleic acid test sample from said subject; and
(c) analyzing the hybridization pattern of said probes to said nucleic acid test sample,
whereby aberrations in the hybridization of said probes to said nucleic acid test sample, as compared to a normal nucleic acid sample, indicates that said subject is at risk for the recurrence of cancer.

30. A method for identifying a subject at risk for metastatic cancer comprising:
(a) providing probes for 1q21, 3p22.1, 9p21.3, 10q22, cen17, and one or more of cen1, cen3, cen9 and cen10;
(b) contacting said probes with a nucleic acid test sample from said subject; and
(c) analyzing the hybridization pattern of said probes to said nucleic acid test sample,
whereby aberrations in the hybridization of said probes to said nucleic acid test sample, as compared to a normal nucleic acid sample, indicates that said subject is at risk for metastatic cancer.

31. A method for predicting cancer progression in a subject comprising:
(a) providing probes for 3p22.1, 1q21, 10q22, 9p21.3, cen17, and one or more of cen1, cen3, cen9, and cen10;
(b) contacting said probes with a nucleic acid test sample from said subject; and
(c) analyzing the hybridization pattern of said probes to said nucleic acid test sample,
whereby aberrations in the hybridization of said probes to said nucleic acid test sample, as compared to a normal nucleic acid sample, indicates that said subject will suffer from progressive cancer.

32. The method of claim 31, wherein a patient having alterations in 10q22 is treated with chemotherapy.

33. A method for distinguishing cancer from granulomatous disease in a subject comprising:
(a) providing probes for 3p22.1, 1q21, 9p21.3, 10q22, cen17, and one or more of cen1, cen3, cen9, and cen10;
(b) contacting said probes with a nucleic acid test sample from said subject;
(c) analyzing the hybridization pattern of said probes to said nucleic acid test sample; and
(d) comparing the hybridization patterns in nucleic acids from two more more cell types represented in said nucleic acid test sample, said two or more cell types comprising neutrophils and epithelials cells,
whereby aberrations in the hybridization of said probes to said neutrophil nucleic acids in said nucleic test sample indicates granulomatous disease, and aberrations in the hybridization of said probes to said epithelial cell nucleic acids in said nucleic test sample indicates cancer.

34. A method of identifying a subject to be segregated from a high cancer risk environment comprising:
(a) providing probes for 3p22, 1q21, 9p21.3, 10q22, cen17, and either cen1, cen3 or cen9, cen10;
(b) contacting said probes with a nucleic acid test sample from said subject; and
(c) analyzing the hybridization pattern of said probes to said nucleic acid test sample,
whereby aberrations in the hybridization of said probes to said nucleic acid test sample, as compared to a normal nucleic acid sample, indicates that said subject should be segregated from said high cancer risk environment.

35. A method of identifying a subject who may be at lower risk to develop cancer of the aerodigestive tract and therefore may continue to use tobacco products comprising:
(a) providing probes for 3p22.1, 1q21, 9p21.3, 10q22, cen17, and one or more of cen1, cen3 cen9, and cen10;
(b) contacting said probes with a nucleic acid test sample from said subject; and
(c) analyzing the hybridization pattern of said probes to said nucleic acid test sample,
whereby a lack of aberrations in the hybridization of said probes to said nucleic acid test sample, as compared to a low-risk nucleic acid sample, indicates that said subject may continue to use tobacco products.

36. A method of assessing a cancer therapy or chemopreventative therapy in a subject comprising:
(a) providing probes for 3p, 10q and cen3;
(b) contacting said probes with a nucleic acid test sample derived from epithelial cells and neutrophils from said subject; and
(c) assessing the amount of aneusomies on chromosome 10, 3p deletions and aneusomies of centromeric 3;
wherein a decrease in the amount of aneusomies in 10q, 3p deletions and aneusomies of centromeric 3 indicates an effective treatment or prevention.
